(19) United States
(12) Patent Application Publication
(10) Pub. No.: US 2005/0272050 A1
(43) Pub. Date: Dec. 8, 2005

(54) CD40 splice variants, compositions for making and methods of using the same

(76) Inventors: Liat Mintz, East Brunswick, NJ (US); Jeanne Bernstein, Yona (IL); Dani Eshel, Kirkur (IL); Amir Toporik, Azur (IL); Aviva Chen, Ramat Gan (IL)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/924,074

(22) Filed: Aug. 23, 2004

Related U.S. Application Data
(63) Continuation-in-part of application No. PCT/IB03/00665, filed on Feb. 24, 2003.
(60) Provisional application No. 60/358,877, filed on Feb. 22, 2002.

(51) Int. Cl. C12Q 1/68; C07H 21/04; C07K 14/74; C07K 16/28; A61K 48/00

(52) U.S. Cl. 435/6; 435/69.1; 435/320.1; 435/325; 530/350; 530/388.22; 514/44; 536/23.5

(57) ABSTRACT

Provided are substantially pure CD40 splice variants that include altered internal sequences and unique tail sequences relative to previously described CD40 protein sequences. Also provided are fragments of the CD40 splice variants comprising at least 10 amino acids including at least 4 amino acids of the unique tail sequence; the unique tail sequences, and homologues thereof having at least 10 amino acids and 90% identity and antibodies which bind to an epitope on such proteins are disclosed. Pharmaceutical compositions comprising the protein, antibodies, isolated nucleic acid molecule that encode such proteins and pharmaceutical composition comprising such nucleic acid molecules are also disclosed. The present invention additionally relates to recombinant expression vectors that include the nucleic acid molecules and host cells which comprise such recombinant expression vectors are disclosed. In vitro methods of detecting in a sample the presence and/or quantity of such proteins or transcript which encodes such proteins are disclosed as are kits and reagents for performing the methods. Methods of modulating CD40-CD154 interactions in an individual are disclosed.

### RELATED APPLICATIONS

This application claims priority to PCT/IB03/00665, filed Feb. 24, 2003, which claims priority to U.S. Ser. No. 60/358,877, filed Feb. 22, 2002. The contents of these applications are incorporated by reference in their entireties.

### FIELD OF THE INVENTION

The invention relates to the identification of CD40 splice variants proteins, the identification and cloning of nucleic acid molecules that are splice variants of CD40, methods of making and using the same and protein and nucleic acid fragments.

### BACKGROUND OF THE INVENTION

CD40 was originally described as a receptor responsible for the activation and differentiation of B-lymphocytes. This receptor engages its ligand (CD154, also named CD40L), promoting cell survival and costimulatory protein expression necessary for interacting with T-lymphocytes. Thus, interaction of B- and T-cells via the CD40-CD154 system allows mutual activation, with B-cells secreting antibodies and T-cells becoming effector cells producing cytokines. Kehry, J. Immunol. 156:2345-2348 (1996).

The CD40-CD154 system has wider implications beyond activating B- and T-lymphocytes. See Schonbeck & Libby, Cell. Mol. Life Sci. 58:4-43 (2001). CD40 is also expressed by migratory immune cells, such as macrophages and dendritic cells, which present antigen and activate T-lymphocytes. Engagement of CD40 by T-lymphocyte CD154 activates these immune cells to express new immune modulators, such as the cytokines IL-1, Il-12 and TNFα. Van Kooten & Banchereau, J. Leukoc. Biol. 67:2-17 (2000).

Recent studies reveal that non-hematopoietic cells, including fibroblasts, endothelial cells, smooth muscle cells and some epithelial cells, constitutively display CD40 on their surface (Schonbeck & Libby, supra), and that this expression is upregulated following exposure to IFNγ. Activation of CD40 signaling in non-hematopoietic cells via CD154 results in new cellular functions, including synthesis of pro-inflammatory cytokines (van Kooten & Banchereau, supra). CD40 engagement on human fibroblasts and endothelial cells induces synthesis of cyclooxygenase (COX-2) and production of prostaglandins. CD40 engagement on endothelial and vascular smooth muscle cells induces synthesis of matrix matalloproteinases (MMP). These enzymes degrade collagens and other connective tissue proteins crucial for the stability of atherosclerotic plaques and their fibrous caps.

It was initially thought that CD154 was only expressed on the surface of T-lymphocytes after their activation. However, eosinophils and mast cells were also found to express CD154. Schonbeck & Libby, supra. Human platelets also contain CD154. Once activated by thrombin or other mediators, platelet internal stores of CD154 are exported to the surface and partially secreted. Hen et al., Nature 391:591-594 (1998). Several other cell types are now known to store CD154, including macrophages, B-lymphocytes, endothelial cells and smooth muscle cells.

A number of pathological processes of chronic inflammatory diseases in humans, and several experimental animal models of chronic inflammation, were shown to be dependent upon or involve the CD40-CD154 system. These include graft-versus-host disease, transplant rejection, neurodegenerative disorders, atherosclerosis, pulmonary fibrosis, autoimmune diseases such as lupus nephritis, systemic lupus erythematosus, rheumatoid arthritis, multiple sclerosis, as well as hematological malignancies and other cancers. A remarkable spectrum of chronic inflammatory conditions can be blocked or substantially reduced by disrupting the CD40-CD154 system. These studies typically employ either mice with targeted disruption of CD40 or CD154 genes, or use neutralizing monoclonal anti-CD154 antibodies. van Kooten & Banchereau, supra. These antibodies putatively work by disrupting the communication bridge constructed by CD40-CD154. The animals in these experimental models appear unaffected by having this system disrupted.

At least two different companies are testing anti-human CD154 antibodies for efficacy in diseases such as systemic lupus erythematosus, graft-versus-host disease, and tissue transplantation.

The success of monoclonal antibodies in disrupting the CD40-CD154 pathway and blunting acute and chronic inflammation suggests that blocking this pathway has therapeutic utility. Additional reports of agonistic anti-CD40 antibodies reducing the progression and severity of rheumatoid arthritis in a murine model (Mauri et al., Nat. Medicine 6:673-679 (2000)), suggest that activating agents of this pathway may also be used in therapy of pathological cases of chronic inflammation. Zanelli & Toes, Nat. Medicine 6:629-630 (2000).

A critical role for CD40-CD154 has been established for several autoimmune diseases, including lupus nephritis, systemic lupus erythematosus, rheumatoid arthritis, and multiple sclerosis. Kobata et al., Rev. Immunogenetics 2:74-80 (2000). Treating such diseases by blocking the costimulatory pathway involving CD40-CD 154 is currently being tested. Goodnow, Lancet 357:2115-2121 (2001). Studies using several animal models of autoimmune diseases show that disease symptoms can be blocked or substantially reduced by disrupting the CD40-CD154 system. Particularly encouraging are reports showing that concurrent therapy with anti-CD154 and CTLA4-Ig (a soluble fusion protein between an homologue of the costimulatory molecule CD28 and the Fc portion of IgG1) had dramatic synergistic effects that not only block disease and inhibit autoantibody production, but also prevent clonal expansion of autoreactive T-cells. Griggs et al., J. Exp. Med. 183:801-810 (1996); Daikh et al., J. Immunol. 159:3104-3108 (1997). These results emphasize the potential value of combining agents that target distinct molecular pathways in immune-mediated diseases.

The involvement of CD40-CD154 in lupus nephritis and SLE has been extensively investigated. Crow & Kirou, Curr. Opin. Rheumatol. 13:361-369 (2001). Several models of murine lupus have been used to investigate the potential therapeutic efficacy of interrupting the CD40-CD154 system, and all have shown impressive inhibition of autoantibody production and nephritis, as well as improved survival. Early et al., J. Immunol. 157:3159-3164 (1996); Daikh et al., supra; Kalled et al., J. Immunol. 160:2158-2165 (1998). Concurrent therapy with anti-CD154 and CTLA4-Ig showed dramatic synergistic effects that lasted long after treatment was discontinued. Daikh et al., supra. Particularly encouraging are the findings that treated mice maintained the ability to mount an effective immune response after completing therapy.

Phase I clinical trials with anti-CD154 were carried out in patients with SLE. Davis et al., Arthritis Rheum. 42:S281 (1999); Davis et al., J. Rheumatol. 28: 95-101 (2001); Davidson et al., Arthritis Rheum. 43: S271 (2000); Kalunian et al., Arthritis Rheum. 43: S271 (2000). These studies indicated that the agent was well-tolerated. However, in another study, thromboembolic complications were reported, due perhaps to the particular agent used. Kawai et al., Nat. Med. 6:114 (2000).

The synovial tissue in RA patients is enriched with mature antigen presenting cells (APCs) and many lymphocytes. Interactions and signaling through the costimulatory CD40-CD154 and CD28-CD80/86 molecules are involved in the initiation and amplification of the inflammatory reactions in the synovium. Haraoui et al., Curr. Pharma. Biotech. 1: 217-233 (2000); Aarvak & Natvig, Arthritis Res. 3: 13-17 (2001). Thus, blocking such signaling pathways might provide a specific immunotherapeutic approach for the treatment of RA. Indeed, prevention of collagen-induced arthritis (CIA), a murine model for RA, was observed upon administration of anti-CD154 antibody. Durie et al., Science 261: 1328-1330 (1993). Treatment with anti-CD154 also prevented arthritis development in a model of immunoglobulin-mediated arthritis.

CD40-CD154 interactions play a critical role in T-cell priming, and are involved in tolerance induction. Ample experimental evidence demonstrates that anti-CD154 antibodies are potent inhibitors of allograft rejection in many diverse transplant models. Kirk et al., Phil. Trans. R. Soc. Lond. 356: 691-702 (2001). The efficacy of anti-CD154 therapy in rodent allografts, e.g., skin, cardiac, islet and bone marrow, all showed that a brief course of therapy at the time of transplantation led to prolonged or indefinite allograft survival. Treatment with CTLA-Ig was synergistic with anti-CD154 therapy. Larsen et al., Nature 381: 434-438 (1996). In non-human primates, treatment with anti-CD154 has been remarkably successful in preventing acute renal allograft rejection. Kirk et al., Nature Med. 5: 686-693 (1999); Larsen et al., Transplantation 69: S123 (2000). In this system, anti-CD154 appears capable of preventing allograft rejection and establishing a long lasting state of donor-specific hyporesponsiveness that is not dependent on continuous immunosuppressive medication. Anti-CD154 therapy was also shown to prevent islet cell rejection (Kenyon et al., Diabetes 48: 1473-1481 (1999); Kenyon et al., Proc. Natl. Acad. Sci. USA 96: 8132-8137 (1999)) and prolong cardiac allograft survival (Pierson et al., Transplantation 68: 1800-1805 (1999)) in non-human primates. The durability of anti-CD154 therapy was very impressive when compared with conventional immunosuppression.

Allogeneic bone marrow transplantation is frequently performed for the treatment of haematological malignancies and aplastic anaemia. However, graft-versus-host disease (GVHD) is still the major complication of this procedure, resulting in immune deficiency, infection, organ damage and leading occasionally to patient death. Blocking strategies of co-stimulatory signals, including CD40-CD154, are being evaluated as targets of therapeutic intervention for GVHD. Simpson, Expert Opin. Pharmacother. 2: 1109-1117 (2001); Tanaka et al., Ann. Hematol. 79: 283-290 (2000). Treatment with sublethal radiation and anti-CD154 antibody prevented GVHD in mice receiving allogeneic bone marrow cells. These mice accepted donor-origin, but not third party skin allografts. Seung et al., Blood 95: 2175-2182 (2000). An ex-vivo approach has been described, in which the blockade of the CD40-CD154 interactions by anti-CD154 induces donor bone marrow cells to become tolerant to host alloantigens, and prevents GVHD in mice. Blazar et al., J. Clin. Invest. 102: 473-482 (1998). In addition, a similar approach led to donor-specific tolerance to secondary skin grafts. Durham et al., J. Immunol. 165: 1-4 (2000).

Atherosclerosis is a leading cause of cardiovascular disease, and the most prevalent cause of death in the western world. Recently, atherosclerosis has been associated with chronic inflammation, linking it to the immune system. The presence of CD154 on platelets and the known ability of platelet-bound CD154 to activate endothelial cells, suggest that a critical role may be to initiate chemotactic and adhesion signals at the site of vascular trauma. An emerging body of evidence supports a key role for the CD40-CD154 system in atheroma progression. Phipps et al., Curr. Opin. Invest. Drugs 2: 773-777 (2001).

Recent data from experimental animal models of atherosclerosis, show that disruption of the CD40-CD154 pathway can prevent atherosclerotic progression and may reverse established lesions. Mach et al., Nature 394: 200-203 (1998); Lutgens et al., Nature Med. 5: 1313-1316 (1999); Lutgens et al., Proc. Natl. Acad. Sci. USA 97: 7464-7469 (2000); Schonbeck et al., Proc. Natl. Acad. Sci. USA 97: 7458-7463 (2000). Blockade of this pathway by this and other biological molecules may prove valuable in the treatment of atherosclerosis. Clinical trials are being currently conducted to ascertain the utility of disrupting CD40-CD154 interactions in human disease.

In most organs, tissue injury is followed by cycles of inflammation and repair. When injury is repetitive or larger in magnitude, this frequently results in scarring or fibrosis. Fibrogenic pathologies are a characteristic feature of a wide spectrum of diseases in many organ systems. Tissue fibrosis can lead to significant organ dysfunction and resulting patient mortality.

There is increasing evidence that generation of specific cytokine patterns by immune and structural cells, and interactions between these cells via the CD40-CD 154 pathway, may mediate many of the key events involved in fibrogenesis. Sime & O'Reilly Clin. Immunol. 99: 308-319 (2001). Following acute injury, both infiltrating platelets and inflammatory cells can activate a variety of local structural cells, including fibroblasts, through the CD40-CD154 system. This interaction triggers production of proinflammatory cytokines, expression of cell adhesion molecules, and induction of cyclooxygenase 2 (COX-2), leading to a pro-fibrogenic response. Thus, interruption of the CD40-CD154 system in acute injury, might reduce inflammation and avoid progression to end-stage fibrosis. Indeed, use of anti-CD154 was effective in protecting against injury and fibrosis in two mouse models: hyperoxic lung injury and radiation-induced lung injury. Adawi et al., Clin. Immunol. Immunopathol. 89: 222-230 (1998); Adawi et al., Am. J. Pathol. 152: 651-657 (1998).

CD40 upregulation is involved in pathogenic cytokine production in patients with inflammatory bowel diseases (IBD). Increased expression of CD40 in B-lymphocytes, monocytes and dendritic cells is observed in patients with ulcerative colitis and Crohn's disease. Sawada-Hase et al., Am. U. Gastroenterol. 95: 1516-1523 (2000); Vuckovic et al., Am. J. Gastroenterol. 96: 2946-2956 (2001). Expression of CD40 and CD154 in B-cells/macrophages and CD4⁺ T cells, respectively, was significantly increased in inflamed mucosa from these patients. Liu et al., J. Immunol. 163: 4049-4057 (1999). Blocking the CD40-CD154 pathway with anti-CD154 antibody in a chronic murine colitis model ameliorates symptoms even after onset of disease. DeJong et al., Gastroenterology 119: 715-723 (2000); Liu et al., J. Immunol. 164: 6005-6014 (2000). Thus, blockade of CD40-CD154 interactions may have therapeutic effects for IBD patients.

The CD40-CD154 system plays a critical role in the response of the immune system to an invading pathogen, leading to an antigen-driven lymphoproliferative process. When downregulation of this tightly controlled mechanism is impaired, lymphoproliferative disorders may occur. CD40 expression is elevated in malignant B- and T-cell lymphomas, and in Reed-Sternberg cells of Hodgkin's disease. CD154 is constitutively expressed in several types of B-cell lymphoid malignancies. Fiumara & Younes, Br. J. Haematol. 113: 265-274 (2001). Furthermore, approximately 50% of patients with these malignancies have elevated levels of biologically active soluble CD154 in their serum. Younes et al., Br. J. Haematol. 100: 135-141 (1998). The effect of CD40 activation in B-cell malignancies has been examined extensively by use of activating anti-CD40 antibodies or soluble CD154. Whenever primary human malignant B-cells were analyzed, CD40 activation consistently enhanced malignant cell survival and mediated their resistance to chemotherapy.

Taken together, the coexpression of CD40 and CD154 by malignant B-cells, the presence of soluble CD154 in the sera of these patients, and the ability of CD40 activation to enhance malignant B-cell survival, suggest that CD40/CD154 may provide an autocrine/paracrine survival loop for malignant B-cells. Thus, interrupting CD40/CD154 interaction may be of therapeutic value in patients with B-cell lymphoid malignancies. Anti-CD154, but surprisingly also stimulatory antibodies to CD40, were successfully tested as immunotherapy for malignant B cell tumors in murine models. French et al., Nat. Med. 5: 548-553 (1999); Schultze & Johnson, Lancet 354: 1225-1227 (1999).

Elevated expression of CD40 was described in other forms of cancer, including epithelial neoplasia, nasopharyngeal carcinoma, osteosarcoma, neuroblastoma and bladder carcinoma. Recombinant soluble CD154 inhibited the growth of CD40(+) human breast cell lines in vitro, due to increased apoptosis. In addition, treatment of tumor-bearing mice with this molecule increased survival rates. Hirano et al., Blood 93: 2999-3007 (1999).

Another aspect of CD40/CD154 in the treatment of malignancies is the potential use of CD154 in immune gene therapy, since CD40/CD154 interaction has been shown to be critical for generating protective T cell-mediated anti-tumor response. In this approach, CD154 is transferred ex vivo into neoplastic cells, by infection with a modified adenovirus. Kipps et al., Sem. Oncol. 27 (suppl 12): 104-109 (2000). The results of a Phase I study in CLL patients show induction of autologous cytotoxic T-cells capable of destroying the neoplastic B cells, concomitant with significant reduction in leukemic cell counts and lymph node sizes. Furthermore, this approach appears to enhance antibody-dependent cellular cytotoxicity, and thereby augment the activity of anti-tumor monoclonal antibody therapy. Wierda et al., Blood 96: 2917-2924 (2000). Thus, this approach alone or in combination with tumor-specific Mab therapy (such as Rituxan, anti-CD20), may offer an effective strategy for the treatment of B-cell malignancies. Transduction of tumor cells ex vivo with CD154, in solid tumors such as neuroblastoma and squamous cell carcinoma, can induce immune responses against the tumor cells, mediating rejection or impeding tumor growth.

Activated T-lymphocytes not only express cell membrane-associated CD154, but also soluble CD154. The kinetics of soluble CD154 (sCD154) expression resemble those observed for the membrane-associated form, though the mechanisms of generation and/or release of sCD154 remain poorly understood. Several studies suggest that it retains the ability to ligate CD40. Recently, the soluble forms of CD154 have received more attention, particularly in association with certain human diseases. Enhanced levels sCD154 have been detected in patients with disorders such as active SLE, Kato et al., J. Clin. Invest. 104: 947-955 (1999), unstable angina, Aukurst et al., Circulation 100: 614-620 (1999), and B-Cell lymphoma. Younes et al., supra.

Soluble CD40 (sCD40) was detected in culture supernantants from CD40-positive cell lines, but not from CD40-negative cells. A substantial proportion of the sCD40 in these cultures retained its ligand binding activity. Bjorck et al., Immunol. 83: 430-437 (1994). High levels of sCD40 were also observed in supernantants of AIDS-related lymphoma B-cell lines. De Paoli et al., Cytometry 30: 33-38 (1997). Expression of sCD40 by B cells was shown to bind CD154 on activated T cells and thought to regulate CD40-CD154 in a negative fashion. Van Kotten et al., Eur. J. Immunol. 24: 787-792 (1994). sCD40 was also detected in serum and urine of healthy donors, and was highly elevated in patients with impaired renal function, including chronic renal failure, haemodialysis and chronic ambulatory peritoneal dialysis (CAPD) patients. Schwabe et al., Clin. Exp. Immunol. 117: 153-158 (1999). Patients with neoplastic disease and chronic inflammatory bowel disease (CIBD) in this study, showed slight but significant elevations of sCD40 in their serum.

A recent study by Tone et al., Proc. Natl. Acad. Sci. 98: 1751-1756 (2001), suggests that sCD40 can be created through alternative splicing. As such, sCD40 molecules may have unique antigenic epitopes, distinct from CD40, which could be used to raise sCD40-specific antibodies.

At least one study suggests that expression of sCD40 regulates CD40-CD154 interactions in a negative fashion. Van Kooten et al., supra. Given the ample evidence for a critical role of CD40-CD154 in injury and inflammation, it appears that targeting this system may prove to play an important therapeutic role in abating inflammation in a variety of diseases. Blocking the CD40-CD154 system could be approached via molecules that act as CD40 antagonists, or that disrupt CD40-CD154 interactions. Reports that agonistic anti-CD40 antibodies can also reduce severity and disease progression, suggest that also activating agents of this pathway may be used in therapy of pathological cases of chronic inflammation.

Monoclonal antibody targeting of the CD40-CD154 pathway has shown beneficial effects in a number of experimental animal models. However, whether these techniques can be applied to humans remains to be determined, since treatment with “humanized” antibodies has obvious limitations. Other options for blocking this pathway with higher specificity and efficacy, such as sCD40, hold promise as therapeutic agents.

### SUMMARY OF THE INVENTION

The present invention relates to substantially pure proteins having the amino acid sequence selected from: SEQ ID NO:2; SEQ ID NO:4; SEQ ID NO:6; SEQ ID NO:7; SEQ ID NO:8; SEQ ID NO:9; SEQ ID NO:10; fragments thereof comprising at least ten amino acids including at least four amino acids of the unique tail sequence; and homologues thereof having at least ten amino acids and 90% identity. The present invention also relates to a pharmaceutical composition including one of these proteins and a pharmaceutically acceptable carrier. The present invention also relates to an isolated nucleic acid sequence that encodes such proteins. The present invention also relates to a pharmaceutical composition including one of these nucleic acid molecules and a pharmaceutically acceptable carrier.

The present invention also relates to substantially pure proteins containing the amino acid sequence selected from: SEQ ID NO:2, SEQ ID NO:4, SEQ ID NO:6, SEQ ID NO:7, SEQ ID NO:8, SEQ ID NO:9, SEQ ID NO:10, SEQ ID NO:11; SEQ ID NO:12; SEQ ID NO:13; SEQ ID NO:14; SEQ ID NO:15; SEQ ID NO:16; SEQ ID NO:17; fragments thereof including at least four amino acids; and homologues thereof having 90% identity.

The present invention also relates to a fragment of SEQ ID NOs:2, 4 or 6, wherein the fragment has a length equal to any integer n equal to or between 10 and 50 amino acids, and wherein the amino acid sequence of the fragment is the amino acid sequence of a member of the respective amino acid sequence starting at any amino acid number 187−x; and ending at any amino acid number 188+((n−2)−x), wherein x is any integer between 0 and (n−2) or the end of the respective amino acid sequence, whichever is the lower number.

The present invention further relates to an antibody or an antibody fragment being capable of specifically binding to a CD40 variant polypeptide comprising an amino acid sequence of claim 3, wherein the antibody or antibody fragment does not bind to an epitope consisting of an amino acid fragment of SEQ ID NO:24.

The present invention relates to isolated nucleic acid molecules including the nucleic acid sequence selected from: SEQ ID NO:1; SEQ ID NO:3; SEQ ID NO:5; fragments thereof that encodes at least ten amino acids including at least four amino acids of the unique tail sequence; and homologues thereof having at least ten amino acids and 90% identity. The present invention also relates to a recombinant expression vector including one of these nucleic acid molecules. The present invention also includes a host cell including this recombinant expression vector. The present invention also includes fragments of SEQ ID NOs:1, 3 and 5 wherein the fragments are 12-150, 15-50 or 18-30 nucleotides in length.

The present invention also relates to isolated nucleic acid molecules that encode proteins of the invention, e.g., nucleic acid molecules of SEQ ID NO:1; SEQ ID NO:3; SEQ ID NO:5 and fragments thereof.

The present invention relates to pharmaceutical composition including such nucleic acid molecules.

The present invention relates to recombinant expression vectors that include such nucleic acid molecules and host cells that comprise such recombinant expression vectors.

The present invention relates to isolated antibodies that bind to an epitope on a protein having the amino acid sequence selected from: SEQ ID NO:2; SEQ ID NO:4; SEQ ID NO:6; SEQ ID NO:7; SEQ ID NO:8; SEQ ID NO:9; SEQ ID NO:10; and fragments thereof, including at least 4 amino acids of the unique tail sequence; and homologues thereof, wherein the antibody does not bind to an epitope consisting of an amino acid fragment of SEQ ID NO:24. The present invention also relates to isolated antibodies to these epitopes having at least ten amino acids and 90% identity to their respective SEQ ID NO. In a preferred embodiment, the isolated antibody is a monoclonal antibody. In a preferred embodiment, the present invention relates to isolated antibodies that bind to the bridge fragments of SEQ ID NO:2, SEQ ID NO:4 and SEQ ID NO:6 described above. The present invention also relates to a method of using these antibodies including the steps of contacting one of the antibodies to a sample, determining if the antibody is bound to a protein in the sample, wherein binding of the antibody to protein in the sample is indicative of the presence of one of the above proteins In one embodiment, the sample is a body fluid. In another embodiment, the body fluid is blood. The present invention also includes a kit for detecting the above proteins, which includes a container including one of the above isolated antibodies and another container that includes a positive or negative control.

The present invention relates to isolated antibodies that bind to an epitope on a protein having the amino acid sequence selected from: SEQ ID NO:11; SEQ ID NO:12; SEQ ID NO:13; SEQ ID NO:14; SEQ ID NO:15; SEQ ID NO:16; SEQ ID NO:17; fragments thereof containing at least 4 amino acids; and homologues thereof having 90% identity.

The present invention further encompasses an antibody or an antibody fragment that binds specifically to an amino acid sequence (epitope) present in any of the isolated chimeric amino acid sequences described above.

The present invention relates to in vitro methods of detecting the presence and/or quantity of a protein such as SEQ ID NO:2; SEQ ID NO:4; SEQ ID NO:6; SEQ ID NO:7; SEQ ID NO:8; SEQ ID NO:9; and SEQ ID NO:10; in a sample and kits and reagents for performing the method.

The present invention relates to in vitro methods of detecting the presence and/or quantity of a protein such as SEQ ID NO:11; SEQ ID NO:12; SEQ ID NO:13; SEQ ID NO:14; SEQ ID) NO:15; SEQ ID NO:16; and SEQ ID NO:17; in a sample and kits and reagents for performing the method.

The present invention relates to in vitro or in vivo methods of detecting whether an individual is expressing a protein selected from SEQ ID NO:2; SEQ ID NO:4; SEQ ID NO:6; SEQ ID NO:7; SEQ ID NO:8; SEQ ID NO:9; and SEQ ID NO:10 by detecting a transcript that encodes the protein in a sample from the individual. In one embodiment, the transcript is detected using polymerase chain reaction. In another embodiment, the sample is a body fluid. In another embodiment, the body fluid is blood.

The present invention relates to in vitro methods of detecting whether an individual is expressing a protein selected from SEQ ID NO:11; SEQ ID NO:12; SEQ ID NO:13; SEQ ID NO:14; SEQ ID NO:15; SEQ ID NO:16; and SEQ ID NO:17 by detecting in a sample from the individual a transcript that encodes the protein.

The present invention relates to methods of modulating CD40-CD154 interactions in an individual. The method includes administering to the individual a protein containing the amino acid sequence selected from SEQ ID NO:2; SEQ ID NO:4; SEQ ID NO:6; SEQ ID NO:7; SEQ ID NO:8; SEQ ID NO:9; SEQ ID NO:10; fragments thereof containing at least 10 amino acids including at least 4 amino acids of the unique tail sequence; and homologues thereof having at least 10 amino acids and 90% identity. The protein is administered in an amount effective to modulate CD40-CD154 interactions in the individual. In one embodiment, the individual is suspected of suffering from chronic inflammatory disease. In another embodiment, the individual is suspected of suffering from a condition selected from cancer, artherosclerosis, and acute injury.

The present invention relates to methods of modulating CD40-CD154 interactions in an individual including administering to the individual a nucleic acid molecule that includes a coding sequence that encodes a protein selected from SEQ ID NO:2; SEQ ID NO:4; SEQ ID NO:6; SEQ ID NO:7; SEQ ID NO:8; SEQ ID NO:9; SEQ ID NO:10; wherein the coding sequence is operatively linked to regulatory sequences necessary for the expression in the individual, and wherein the protein is expressed in an amount effective to modulate CD40-CD154 interactions. In one embodiment, the individual is suspected of suffering from chronic inflammatory disease. In another embodiment, the individual is suspected of suffering from a condition selected from the group consisting of cancer, artherosclerosis, and acute injury.

According to another aspect of the present invention there is provided an isolated chimeric polypeptide, including a first amino acid sequence being at least 90% homologous to amino acids 1-187 of wild-type CD40 corresponding to GI:15214587 (SEQ ID NO: 24), and a second amino acid sequence being at least 70%, optionally at least 80%, preferably at least 85%, more preferably at least 90% and more preferably at least 95%, more preferably at least 98% and most preferably at least 99% homologous to a polypeptide having the sequence ESWTMGPGESLGRWELKGEMRHTGTLDGKKGRGGSLGVWYHSSATYLGSLGKSLPLS (SEQ ID NO:11), wherein the first and said second amino acid sequences are contiguous and in a sequential order. According to another aspect of the present invention, there is provided an isolated polypeptide containing a polypeptide having a sequence being at least 70%, optionally at least 80%, preferably at least 85%, more preferably at least 90% and more preferably at least 95%, more preferably at least 98% and most preferably at least 99% homologous to a polypeptide having the sequence

| | |
|---|---|
| ESWTMGPGESLGRWELKGEMRHTGTLDGKKGRGGSL | (SEQ ID NO:11) |
| GVWYHSSATYLGSLGKSLPLS. | |

According to another aspect of the present invention there is provided a bridge fragment of SEQ ID NO:2 between ten and fifty amino acids in length that spans the first and second amino acid sequences described above. This bridge fragment includes a polypeptide having a length “n,” wherein n is at least about ten amino acids in length, optionally at least about twenty amino acids in length, preferably at least about thirty amino acids in length, more preferably at least about forty amino acids in length and most preferably at least about fifty amino acids in length, wherein at least two amino acids include GE, having a structure as follows (numbering according to SEQ ID NO:2): a sequence starting from any of amino acid numbers (187−x) to 187; ending at any of amino acid numbers 188+((n−2)−x), in which x varies from 0 to (n−2).

For example, for peptides of ten amino acids (such that n=10), the starting position could be as “early” in the sequence as amino acid number 179 if x=n−2=8 (i.e., 179=187−8), such that the peptide would end at amino acid number 188 (188+(8−8=0)). On the other hand, the peptide could start at amino acid number 187 if x=0 (i.e., 187=187−0), and could end at amino acid 196 (188+(8−0=8)).

The bridge fragment above may optionally include a polypeptide being at least 70%, optionally at least about 80%, preferably at least about 85%, more preferably at least about 90%, more preferably at least about 95%, more preferably at least 98% and most preferably at least 99% homologous to at least one sequence described above.

Similarly, the bridge fragment may optionally be relatively short, such as from about four to about nine amino acids in length. For four amino acids, the first bridge fragment would include the following peptides: VCGE (SEQ ID NO:38), CGES (SEQ ID NO:39), GESW (SEQ ID NO:40). All peptides feature GE as a portion thereof. Peptides of from about five to about nine amino acids could optionally be similarly constructed.

According to another aspect of the present invention there is provided an isolated chimeric polypeptide, including a first amino acid sequence being at least 90% homologous to amino acids 1-187 of wild type CD40 (SEQ ID NO: 24), and a second amino acid sequence being at least 70%, optionally at least 80%, preferably at least 85%, more preferably at least 90% and most preferably at least 95% homologous to a polypeptide having the sequence LGLE (SEQ ID NO:12), wherein the first and said second amino acid sequences are contiguous and in a sequential order.

According to another aspect of the present invention there is provided an isolated polypeptide, including a polypeptide having the sequence being at least 50%, preferably at least 75% homologous to a polypeptide having the sequence LGLE (SEQ ID NO:12)

According to another aspect of the present invention there is provided a bridge fragment of SEQ ID NO:4 between ten and fifty amino acids in length that spans the first and second amino acid sequences described above. The bridge fragment includes a polypeptide having a length “n”, wherein n is at least about ten amino acids in length, optionally at least about twenty amino acids in length, preferably at least about thirty amino acids in length, more preferably at least about forty amino acids in length and most preferably at least about fifty amino acids in length, wherein at least two amino acids include GL, having a structure as follows (numbering according to SEQ ID NO:4): a sequence starting from any of amino acid numbers 187−x to 187; ending at any of amino acid numbers 188+((n−2)−x), in which x varies from 0 to (n−2), such that the number of the ending amino acid is not larger than 191.

For example, for peptides of ten amino acids (such that n=10), the starting position could be as “early” in the sequence as amino acid number 179 if x=n−2=8 (i.e., 179=187−8), such that the peptide would end at amino acid number 188 (188+(8−8=0)). On the other hand, the peptide could start at amino acid number 182 if x=5 (i.e., 182=187−5), and could end at amino acid 191 (188+(8−5=3)). Since the last amino acid number is 191, the number of the ending amino acid of the bridge portion cannot be larger than 191.

The bridge fragment above can be a polypeptide including a sequence at least 50%, optionally at least about 75%, preferably at least about 85%, more preferably at least about 90% and most preferably at least about 95% homologous to at least one sequence described above.

Similarly, the bridge fragment may optionally be relatively short, such as from about four to about nine amino acids in length. For four amino acids, the first bridge portion would comprise the following peptides: VCGL (SEQ ID NO:41), CGLG (SEQ ID NO:42), GLGL (SEQ ID NO:43). All peptides feature GL as a portion thereof. Peptides of from about five to about nine amino acids could optionally be similarly constructed.

According to another aspect of the present invention there is provided an isolated chimeric polypeptide including a first amino acid sequence being at least 90% homologous to amino acids 1-187 of wild type CD40 (SEQ ID NO: 24), and a second amino acid sequence being at least 70%, optionally at least 80%, preferably at least 85%, more preferably at least 90% and more preferably at least 95%, more preferably at least 98% and most preferably at least 99% homologous to a polypeptide having the sequence ESWTMGPGESLGRSPGSAESPGGDPHHLRDPVCHPLGAGLYQKGGQEANQ (SEQ ID NO: 13), wherein the first and the second amino acid sequences are contiguous and in a sequential order.

According to another aspect of the present invention there is provided an isolated polypeptide including a polypeptide having the sequence being at least 70%, optionally at least 80%, preferably at least 85%, more preferably at least 90% and more preferably at least 95%, more preferably at least 98% and most preferably at least 99% homologous to a polypeptide having the sequence

| | |
|---|---|
| ESWTMGPGESLGRSPGSAESPGGDPHHLRDPVCHPL | (SEQ ID NO:13) |
| GAGLYQKGGQEANQ. | |

According to another aspect of the present invention there is provided a bridge fragment of SEQ ID NO: 6 between ten and fifty amino acids in length that spans the first and second amino acid sequences described above. The bridge fragment comprises a polypeptide having a length “n”, wherein n is at least about ten amino acids in length, optionally at least about twenty amino acids in length, preferably at least about thirty amino acids in length, more preferably at least about forty amino acids in length and most preferably at least about fifty amino acids in length, wherein at least two amino acids comprise GE, having a structure as follows (numbering according to SEQ ID NO:6): a sequence starting from any of amino acid numbers 187−x to 187; ending at any of amino acid numbers 188+((n−2)−x), in which x varies from 0 to n−2, such that the number of the ending amino acid is not larger than 237.

For example, for peptides of ten amino acids (such that n=10), the starting position could be as “early” in the sequence as amino acid number 179 if x=n−2=8 (i.e., 179=187−8), such that the peptide would end at amino acid number 188 (188+(8−8=0)). On the other hand, the peptide could start at amino acid number 187 if x=0 (i.e., 187=187−0), and could end at amino acid 196 (188+(8−0=8)).

The bridge fragment above may optionally include a polypeptide being at least 70%, optionally at least 80%, preferably at least 85%, more preferably at least 90% and more preferably at least 95%, more preferably at least 98% and most preferably at least 99% homologous to at least one sequence described above.

Similarly, the bridge fragment may optionally be relatively short, e.g., from about four to about nine amino acids in length. For four amino acids, the first bridge portion would include the following peptides: VCGE (SEQ ID NO:38), CGES (SEQ ID NO:39), GESW (SEQ ID NO:40). All peptides feature GE as a portion thereof. Peptides of from about five to about nine amino acids could optionally be similarly constructed.

The present invention also relates to pharmaceutical composition including such proteins.

The present invention also relates to an antibody or an antibody fragment being capable of specifically binding to a CD40 variant polypeptide of the invention. Further, this antibody or antibody fragment does not bind to an epitope consisting of an amino acid fragment of SEQ ID NO:24.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 shows a representation of the CD40 splice variants NJ1, NJ2, and NJ3 at the RNA level. The white box represents a unique sequence. The numbered arrows represent the primers that were used with their Sequence identification number.

FIG. 2 shows a schematic representation of the CD40 splice variants NJ1, NJ2, and NJ3 at the protein level.

FIG. 3 shows 2% agarose gel analysis for the NJ1, NJ2 and NJ3 splice variants of CD40. The NJ1 variant is amplified with a forward “cd40 general primer” (SEQ ID NO:18) and a reverse “cd40nj1 primer” (SEQ ID NO:19), resulting in an expected band of about 290 base pairs. The NJ2 variant is amplified with a forward “cd40 general primer” (SEQ ID NO:18) and a reverse “cd40nj2 primer” (SEQ ID NO:20), resulting in an expected band of about 260 base pairs. The NJ3 variant is amplified with a forward “cd40nj3 primer” (SEQ ID NO:22) and a reverse “cd40nj3 primer” (SEQ ID NO:21), resulting in an expected band of about 220 base pairs. The lanes, from right to left, represent the following templates: Marker, K562 (ATCC: CCL-243), NL564, bone marrow (Clontech, Cat # 1110932), thymus (Clontech, Cat # 1070319) and spleen (Ambion, Cat # 111PO106B). In NJ3 a weak band in colon tissue is also detected (Ichilov Hos., Cat # CG335; the left side lane).

FIG. 4 shows Western blots with anti-NJ1 (FIG. 4A) and anti-NJ3 (FIG. 4B) antibodies.

FIG. 5 shows a competition assay, demonstrating Elisa binding of the CD40 NJ1 and NJ3 variants to CD154 ligand, as compared to positive control of WT CD40 (non-labeled) and to negative control of mock, as described in Example 5.

FIG. 6 shows the FACS analysis: binding of CD40 NJ1 and NJ3 variants to mouse fibroblasts that do not express human CD154, as compared to CD40 skipping 6 variant (U.S. Pat. No. 6,720,182 and U.S. patent application No. 60/547,422, which is incorporated herein by reference), wild type CD40-FC tagged and EA5, as described in Example 6.

FIG. 7 shows the FACS analysis: binding of CD40 NJ1 variant to mouse fibroblasts that were transfected with human CD154, as described in Example 6.

FIG. 8 shows the FACS analysis of CD40 variants NJ1 binding to mouse fibroblasts transfected with CD154-Isotype control, as described in Example 6.

FIG. 9 shows the FACS analysis of CD40 variants NJ3 binding to mouse fibroblasts transfected with CD154, as described in Example 6.

FIG. 10 shows the FACS analysis of CD40 variants NJ3 binding to mouse fibroblasts transfected with CD154-Isotype control, as described in Example 6.

FIG. 11 shows the control FACS analysis of CD40 variants NJ3 binding to mouse fibroblasts not expressing CD154, as described in Example 6.

FIG. 12 shows the mock control FACS analysis of CD40 variants NJ1 and NJ3 binding to mouse fibroblasts transfected with CD154, as described in Example 6.

FIG. 13 shows the FACS analysis of CD40 variants skipping 6 binding to mouse fibroblasts transfected with CD154, as described in Example 6.

FIG. 14 shows the FACS analysis of CD40 skipping 6 variant binding to mouse fibroblasts transfected with CD154-Isotype control, as described in Example 6.

FIG. 15 shows the mock control FACS analysis of CD40 skipping 6 variant binding to mouse fibroblasts transfected with CD154, as described in Example 6.

FIG. 16 shows the control FACS analysis of CD40 skipping 6 variant binding to mouse fibroblasts not expressing CD154, as described in Example 6.

FIG. 17 shows positive control: FACS analysis of CD40 skipping 6-FC variant binding to mouse fibroblasts transfected with CD154, as described in Example 6.

FIG. 18 shows isotype control FACS analysis of CD40 skipping 6 variant binding to mouse fibroblasts transfected with CD154, as described in Example 6.

FIG. 19 shows the influence of NJ-Mock treatment on RANTES secretion (control), as described in Example 7.

FIG. 20 shows the influence of CD40 NJ1 variant protein treatment on RANTES secretion, as described in Example 7.

FIG. 21 shows the influence of CD40 NJ3 variant protein treatment on RANTES secretion, as described in Example 7.

### DETAILED DESCRIPTION OF PREFERRED EMBODIMENTS

The invention is broadly drawn to CD40 splice variant proteins and methods of using these proteins, for example, for the detection of CD154 and the modulation of CD40 signaling. For example, CD40 splice variant proteins of the present invention contain a signal peptide but lack a transmembrane domain present in previously described CD40 proteins. Consequently, the CD40 splice variant proteins are secreted from the cell and are not cell-associated. While not wishing to be bound by theory, it is postulated that by maintaining the ligand-binding domain, the splice variant proteins bind CD154, the ligand of the native CD40 receptor. Thus, the splice variant proteins act as a competitive inhibitor of the native CD40 receptor, and the CD40 splice variant proteins modulate the activity of the native CD40 receptor.

The splice variant polypeptides are useful for disrupting the CD40-CD154 system in vitro and in vivo. The splice variant proteins are also useful for treating a variety of chronic inflammatory diseases including, e.g., graft-versus-host disease, transplant rejection, neurodegenerative disorders, atherosclerosis, pulmonary fibrosis, autoimmune diseases such as lupus nephritis, systemic lupus erythematosus, rheumatoid arthritis, multiple sclerosis, as well as hematological malignancies and other cancers.

Because the splice variant proteins bind CD154 in vitro (see, e.g., Examples 5 and 6), the splice variant proteins are also useful for detecting and, if desired, localizing CD154 in a biological sample. Among the advantages of using the CD40 splice variants disclosed herein is their relative ease in synthesis and isolation as compared to antibody reagents, which typically must be recovered from immune sera and characterized, or recovered from hybridomas producing CD154-reactive antibodies.

Definitions

As used herein the term “unique tail” is meant to refer to the amino acid sequence at the C terminus of each CD40 splice variant which differs from the amino acid sequence at the C terminus of wild type CD40. The unique tail region of the CD40 splice variant SEQ ID NO:2 includes the last 57 amino acids, i.e., the 57 amino acids at the C terminus (SEQ ID NO:11). The unique tail region of the CD40 splice variant SEQ ID NO:4 includes the last 4 amino acids, i.e., the 4 amino acids at the C terminus (SEQ ID NO:12). The unique tail region of the CD40 splice variant SEQ ID NO:6 includes the last 50 amino acids, i.e., the 50 amino acids at the C terminus (SEQ ID NO:13). The unique tail region of the CD40 splice variant SEQ ID NO:7 includes the last 21 amino acids, i.e., the 21 amino acids at the C terminus (SEQ ID NO:14). The unique tail region of the CD40 splice variant SEQ ID NO:8 includes the last 42 amino acids, i.e. the 42 amino acids at the C terminus (SEQ ID NO:15). The unique tail region of the murine CD40 splice variant SEQ ID NO:9 includes the last 42 amino acids, i.e. the 42 amino acids at the C terminus (SEQ ID NO:16). The unique tail region of the murine CD40 splice variant SEQ ID NO:10 includes the last 25 amino acids, i.e. the 25 amino acids at the C terminus (SEQ ID NO:17).

As used herein the terms “CD40 splice variants product”, “CD40 splice variant proteins” and “CD40 splice variants” are used interchangeably and meant to refer to an amino acid sequence encoded by a CD40 splice variants nucleic acid sequences which are naturally occurring mRNA sequences obtained as a result of alternative splicing, and fragments and homologues thereof. The amino acid sequences may be a peptide, a protein, as well as peptides or proteins having chemically modified amino acids such as a glycopeptides or glycoproteins. CD40 splice variants products include SEQ ID NO:2; SEQ ID NO:4; SEQ ID NO:6; SEQ ID NO:7; SEQ ID NO:8; SEQ ID NO:9; and SEQ ID NO:10. The terms also refer to homologues of these sequences in which one or more amino acids has been added, deleted, substituted or chemically modified as well as fragments of these sequences having at least 10 amino acids including at least 4 amino acid residues of the unique tail region. As set forth herein, SEQ ID NO:9 includes a sequence of the unique tail only. The murine CD40 splice variant protein intended to be depicted in SEQ ID NO:9 includes native murine CD40 sequences to the N terminal of the unique tail sequence. Examples of CD40 splice variant protein intended to be depicted in SEQ ID NO:9 include those murine proteins with the unique tail set forth linked to the non-unique tail regions of CD40 such as the non-unique tail region of SEQ ID NO:10.

As used herein the term “CD40 splice variants nucleic acid molecule” is meant to refer to a nucleic acid molecule that encodes a CD40 splice variant. Accordingly such CD40 splice variants nucleic acid molecules include nucleic acid molecules that encode naturally-occurring CD40 splice variants, particularly those naturally occurring mRNA sequences obtained as a result of alternative splicing. CD40 splice variants nucleic acid molecules include nucleic acid molecules that encodes fragments of CD40 splice variants and homologues thereof. CD40 splice variant nucleic acid molecules include SEQ ID NO:1, SEQ ID NO:3, and SEQ ID NO:5. The term also refers to homologues of these sequences which encode amino acid sequences in which one or more amino acids has been added, deleted, substituted or chemically modified as well as fragments of these sequences which encode amino acid sequences having at least 10 amino acids including at least 4 amino acids of the unique tail region.

As used herein, the term “fragments” as applied to protein fragments of CD40 splice variants refers to those fragments which include at least 4 amino acids of the unique tail regions (SEQ ID NO:11, SEQ ID NO:12, SEQ ID NO:13, SEQ ID NO:14, SEQ ID NO:15, SEQ ID NO:16 or SEQ ID NO:17). In some preferred embodiments the fragment includes 5, 6, 7, 8, 9 or 10 amino acids of the unique tail region of SEQ ID NO:2, SEQ ID NO:6, SEQ ID NO:7, SEQ ID NO:8, SEQ ID NO:9, and SEQ ID NO:10. In some preferred embodiments the fragment includes 11, 12, 13, 14, 15, 16, 17, 18, 19, 20 or 21 amino acids of the unique tail region of SEQ ID NO:2, SEQ ID NO:6, SEQ ID NO:7, SEQ ID NO:8, SEQ ID NO:9, and SEQ ID NO:10. In some preferred embodiments the fragment includes 21, 22, 23, 24 or 25 amino acids of the unique tail region of SEQ ID NO:2, SEQ ID NO:6, SEQ ID NO:8 and SEQ ID NO:9. In some preferred embodiments the fragment includes 26, 27, 28, 29 or 30 amino acids of the unique tail region of SEQ ID NO:2, SEQ ID NO:6, SEQ ID NO:8 and SEQ ID NO:9. In some preferred embodiments the fragment includes 31, 32, 33, 34, 35, 36, 37 or 38 amino acids of the unique tail region of SEQ ID NO:2, SEQ ID NO:6 and SEQ ID NO:9. In some preferred embodiments the fragment includes 39, 40, 41 or 42 amino acids of the unique tail region of SEQ ID NO:2 and SEQ ID NO:9. In some preferred embodiments, the fragment includes 43, 44 or 45 amino acids of the unique tail region of SEQ ID NO:2. In some preferred embodiments, the fragment includes the entire unique tail region. Fragments generally comprise at least 10 amino acids although it is contemplated that smaller fragments may be useful for some purposes.

According to certain embodiments of the invention, the CD40-L binding properties are due to the presence of the following amino acids at positions E74, Y82, N86, D84, E114, E117 of the CD40 (wt and variant, as well), so that the fragment of the invention preferably includes one or more regions containing these amino acids critical for binding CD40 binding domains linked to each other (either in the order appearing in the native protein or in another order), and further linked to said at least four consecutive amino acids of the tail sequence. In another embodiment of the invention, the following amino acids at positions E74, Y82, N86, D84, E114, E117 of the CD40 have either been maintained as in the parent sequence, or substituted by conservative substitution.

Preferably at least 10 consecutive amino acids of the unique tail segment, and most preferably all the amino acids of this unique segment are present in the fragment.

As used herein, the term “fragments” as applied to fragments of nucleic acid molecules, refers to nucleic acid molecules that include coding sequences encoding fragments of the CD40 splice variant as described above.

As used herein the term “nucleic acid sequence” refers to a sequence composed of DNA nucleotides, RNA nucleotides or a combination of both types and may include natural nucleotides, chemically modified nucleotides and synthetic nucleotides.

As used herein the term “amino acid sequence” refers to a sequence composed of one or more naturally occurring amino acids, chemically modified amino acids or synthetic amino acids.

As used herein the term “homologues of variants/products” refers to amino acid sequences of variants, with one or more amino acids added, deleted or replaced. The altered amino acids are in regions where the variant differs from the original sequence.

As used herein the term “conservative substitution” refers to substituting an amino acid in one class with an amino acid of the same class. A class is defined by common physicochemical amino acid side chain properties and high substitution frequencies in homologous proteins found in nature, as determined by, e.g., a standard Dayhoff frequency exchange matrix or BLOSUM matrix. Six general classes of amino acid side chains have been categorized and include: Class 1 (Cys); Class II (Ser, Thr, Pro, Ala, Gly); Class III (Asn, Asp, Gln, Glu); Class IV (His, Arg, Lys); Class V (Ile, Leu, Val, Met), and Class VI (Phe, Tyr, Trp). For example, substitution of an Asp for another class III residue such as Asn, Gln, or Glu, is a conservative substitution.

As used herein the term “non-conservative substitution” refers to substituting an amino acid in one class with an amino acid from another class; for example, substitution of an Ala, a class II residue, with a class III residue such as Asp, Asn, Glu, or Gln.

As used herein the term “chemically modified” when referring to a protein of the invention, refers to a protein with least one of its amino acid residues modified either by natural processes, e.g., processing or other post-translational modifications, or by chemical modification techniques that are well known in the art. Examples of modifications include acetylation, acylation, amidation, ADP-ribosylation, glycosylation, GPI anchor formation, covalent attachment of a lipid or lipid derivative, methylation, myristylation, pegylation, prenylation, phosphorylation, ubiquitination, or any similar process.

As used herein the term “biologically active” refers to the variant product having one or more biological activities, e.g., capability of binding to the CD154 or to other agonists of wild type CD40.

As used herein the term “immunologically active” refers to the capability of a natural, recombinant or synthetic variant product, or any fragment thereof, to induce a specific immune response in appropriate animals or cells and to bind with specific antibodies. Thus, for example, an immunologically active fragment of variant product denotes a fragment which retains some or all of the immunological properties of the variant product, e.g., can bind specific anti-variant product antibodies or which can elicit an immune response which will generate such antibodies or cause proliferation of specific immune cells which produce variant.

As used herein the term “optimal alignment” refers to an alignment providing the highest percent identity score. Such alignment can be performed using a variety of commercially available sequence analysis programs, e.g., the local alignment program LALIGN using a ktup of 1, default parameters and the default PAM. A preferred alignment is the one performed using the CLUSTAL-W program from MACVECTOR™, operated with an open gap penalty of 10.0, an extended gap penalty of 0.1, and a BLOSUM similarity matrix. If a gap needs to be inserted into a first sequence to optimally align it with a second sequence, the percent identity is calculated using only the residues that are paired with a corresponding amino acid residue (i.e., the calculation does not consider residues in the second sequences that are in the gap of the first sequence). In cases of aligning known gene sequences with sequences of new variants, the optimal alignment generally aligns the identical parts of both sequences together, while not aligning the sections of the sequences that differ.

As used herein the term “having at least 90% identity” with respect to two amino acid or nucleic acid sequence sequences, refers to the percentage of identical residues in the two sequences when the sequences are optimally aligned. Thus, 90% amino acid sequence identity means that 90% of the amino acids in two or more optimally aligned polypeptide sequences are identical.

As used herein the term “isolated nucleic acid molecule having a variant nucleic acid sequence” refers to a nucleic acid molecule that includes at least a portion of the CD40 splice variant nucleic acid coding sequence. The isolated nucleic acid molecule may include the CD40 splice variant nucleic acid sequence as an independent insert or it may include the CD40 splice variant nucleic acid sequence fused to an additional coding sequences, encoding together a fusion protein in which the variant coding sequence is the dominant coding sequence (for example, the additional coding sequence may code for a signal peptide). The CD40 splice variant nucleic acid sequence may be in combination with non-coding sequences, e.g., introns or control elements, such as promoter and terminator elements or 5′ and/or 3′ untranslated regions, effective for expression of the coding sequence in a suitable host; or may be a vector in which the CD40 splice variant protein coding sequence is a heterologous.

As used herein the term “expression vector” refers to vectors that have the ability to incorporate and express heterologous DNA fragments in a foreign cell. Many prokaryotic and eukaryotic expression vectors are known and/or commercially available. A recombinant expression vector may be a plasmid, phage, viral particle or other vector and other nucleic acid molecules or nucleic acid molecule containing vehicles useful to transform host cells and which, when introduced into an appropriate host, contains the necessary genetic elements to direct expression of the coding sequence that encodes a CD40 splice variant of the invention. The coding sequence is operably linked to the necessary regulatory sequences. Expression vectors are well known and readily available. Selection of appropriate expression vectors is within the knowledge of those having skill in the art.

As used herein the term “deletion” refers to a change in either nucleotide or amino acid sequence in which one or more nucleotides or amino acid residues, respectively, are absent. Preferably, an amino acid sequence maintains at least 20% of its parental amino acid content. More preferably no more than 10% of the amino acids of an amino acid sequence are deleted and more preferably none of the amino acids are deleted.

As used herein, the term “provided that at least 80% of the amino acids in the parent protein are maintained unaltered in the variants” permits 20% changes by a combination of substitution, chemical modification and deletion, i.e., the same variant may have substitutions, chemical modifications and deletions so long as at least 80% of the native amino acids are identical to those of the native sequence both as regards the nature of the amino acid residue and its position in the sequence. In addition, the properties of the parent sequence, in binding to CD40 ligand (CD154), have to be maintained in the composition, typically at the same or higher level.

Typically “essential amino acids” (essential for binding to the ligand) are maintained or replaced by conservative substitutions while non-essential amino acids may be maintained, deleted or replaced by conservative or non-conservative replacements. Essential amino acids are those indicated under E74, Y82, N86, D84, E114, E117 of the CD40 NJ (1-3) variant, which are at the same positions for the WT CD40.

As used herein, the term, “pharmaceutical composition” includes fragments of, at least, 4 consecutive amino acids of the tail attached to a sub-sequence comprising at least one of the following amino acids: E74, Y82, N86, D84, E114, E117 of the CD40 splice variant.

As used herein the terms “insertion” and “addition” refers to that change in a nucleotide or amino acid sequence that has resulted in the addition of one or more nucleotides or amino acid residues, respectively, as compared to the naturally occurring sequence.

As used herein the term “substitution” is meant to refer to replacement of one or more nucleotides or amino acids by different nucleotides or amino acids, respectively. When referring to amino acid sequences, the substitution may be conservative or non-conservative.

As used herein the term “antibody” refers to complete, intact antibodies, and functional fragments of antibodies such as those without the Fc portion, single chain antibodies, fragments consisting of essentially only the variable, antigen-binding domain of the antibody, etc, as well as Fab fragments and F(ab)₂ fragments. Antibodies include monoclonal antibodies such as murine monoclonal antibodies, chimeric antibodies, primatized antibodies and humanized antibodies or functional fragments thereof. An antibody may be an IgG, IgM, IgD, IgA, and IgG antibody.

As used herein the term “alternative splicing” refers to exon exclusion, and deletion of terminal sequences in the variants, as compared to the original sequence.

As used herein, “an individual is suspected of being susceptible to a particular disease condition or disorder” refers to an individual who is at a elevated risk of developing a particular disease condition or disorder relative to a population. Examples of individuals at a particular risk of developing a particular disease, condition or disorder are those whose family medical history indicates above average incidence of such disease, condition or disorder among family members and/or those who have already developed such disease, condition or disorder and have been effectively treated who therefore face a risk of relapse and recurrence. Advancements in the understanding of genetics and developments in technology, as well as epidemiology, allow for determining the probability and assessing the risk that an individual has for developing certain diseases, conditions or disorders. Using family health histories and/or genetic screening, it is possible to estimate the probability that a particular individual has for developing certain types of diseases, conditions or disorders. Those individuals that have been identified as being predisposed to developing a particular form of disease, condition or disorder can be monitored or screened to detect evidence of such disease, condition or disorder. Upon discovery of such evidence, early treatment can be undertaken to combat the disease, condition or disorder. Similarly, those individuals who have already developed a particular disease, condition or disorder and who have been treated are often particularly susceptible to relapse and reoccurrence. Such individuals can be monitored and screened to detect evidence of disease, condition or disorder and upon discovery of such evidence, early treatment can be undertaken.

As used herein, the term “antibody composition” refers to the antibody or antibodies required for the detection of the protein. For example, the antibody composition used for the detection of the CD40 splice variant protein in a test sample comprises a first antibody that binds the CD40 splice variant protein as well as a second or third detectable antibody that binds the first or second antibody, respectively.

Novel splice variants of the transcript that encodes CD40 have been isolated, characterized and cloned. These splice variants include naturally occurring sequences obtained by alternative splicing of the known wild type CD40 gene depicted as CD40 HUMAN Swiss Prot under Accession Number P25942 which is incorporated herein by reference (SEQ ID NOs: 23 and 24). The novel splice variants of the invention are not merely truncated forms, or fragments of the known gene, but rather novel sequences that naturally occur within the body of individuals. These splice variants include nucleic acid molecules that encode the extracellular region of CD40 or a fragment thereof linked to a unique tail sequence. In some embodiments, the extracellular region is fully conserved while in others there may be deletions, insertions or substitutions. In some preferred embodiments, the translation product of the splice variant is a soluble protein that retains the CD40 function of binding to CD40 ligands such as CD154. In a preferred embodiment, the translation product of the splice variant is a soluble protein that binds to CD40. In another preferred embodiment, the translation product of the splice variant is a soluble protein that binds to both CD40 and CD154.

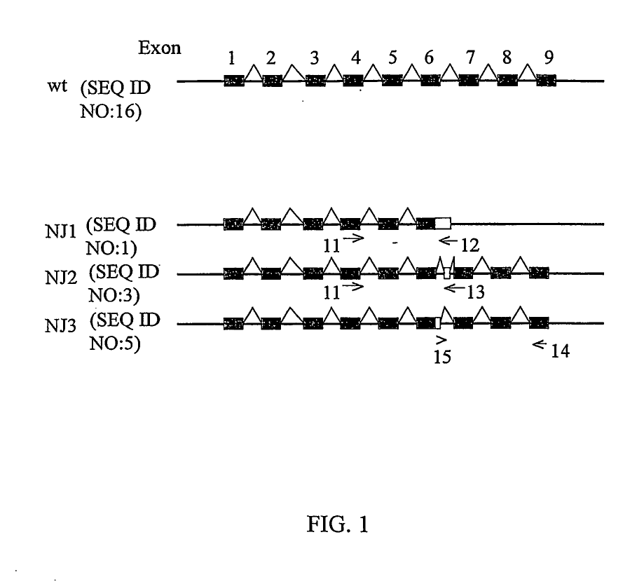

Three splice variants, designated as NJ1, NJ2 and NJ3, have been predicted. FIG. 1 shows these CD40 splice variants at the RNA level.

NJ1 (SEQ ID NO:1)—This splice variant includes exons 1-6 and the intron following exon 6 (FIG. 1). The mRNA is not predicted to contain exons 7, 8 and 9 of the wild type isoform.

NJ1 is predicted to encode a protein that contains 57 unique amino acids in its C-terminus, in addition to the N-terminal 187 amino acids of CD40. The primers that were used to isolate this variant were the “forward cd40 general primer” (SEQ ID NO:18) located in exon 4 (position 400-423 in SEQ ID NO:1), and the “reverse cd40nj1 primer” (SEQ ID NO:19) located in exon 6 (position 677-700 in SEQ ID NO:1).

NJ2 (SEQ ID NO:3)—This splice variant is generated through the addition of a novel exon between exons 6 and 7, that has legitimate splice donor and acceptor sites, causing the premature termination of the protein (FIG. 1). The protein is predicted to contain 4 unique amino acids in its C-terminus, in addition to the N-terminal 187 amino acids of CD40. The primers that were used to isolate this variant were the “forward cd40 general primer” (SEQ ID NO:18) located in exon 4 (position 400-423 in SEQ ID NO:3), and the “reverse cd40nj2 primer” (SEQ ID NO:20) located in exon 7 (position 633-657 in SEQ ID NO:3).

NJ3 (SEQ ID NO:5)—This splice variant was generated through intron retention, and it includes part of the intron following exon 6 (FIG. 1). The alternative splice acceptor used in this case is legitimate. The protein is predicted to contain 50 unique amino acids in its C-terminus, in addition to the N-terminal 187 aa of CD40. The primers that were used to isolate this variant were the “forward cd40nj3 primer” (SEQ ID NO:22) located in exon 6 (position 633-655 in SEQ ID NO:5) and the “reverse cd40nj3 primer” (SEQ ID NO:21), located in exon 9 (position 829-851 in SEQ ID NO:5).

Characteristics of wild type CD40 and splice variants NJ1, NJ2 and NJ3 are shown in Table 1.

TABLE 1

| | WT | NJ1 | NJ2 | NJ3 |
|---|---|---|---|---|
| Number of | 277 | 244 | 191 | 237 |
| amino | | | | |
| acids: | | | | |
| Amino acids | 277 | 1-187 | 1-187 | 1-187 |
| common | | | | |
| with WT | | | | |
| Molecular | 30618 | 26723 | 21045 | 25769 |
| weight (Da): | | | | |
| (+sp) | | | | |
| Molecular | 28258 | 24362 | 18684 | 23408 |
| weight (Da): | | | | |
| (−sp) | | | | |
| Theoretical pI: | 5.49 | 5.58 | 5.01 | 5.12 |
| (+sp) | | | | |
| Theoretical pI: | 5.39 | 5.48 | 4.9 | 5.02 |
| (−sp) | | | | |
| GRAVY | −0.267 | −0.352 | −0.245 | −0.419 |
| (+sp) | | | | |
| GRAVY | −0.369 | −0.476 | −0.395 | −0.554 |
| (−sp) | | | | |
| Tmpred | +++ | +/− | − | − |
| | | Not clear; | | |
| | | however, the | | |
| | | protein was | | |
| | | shown to be | | |
| | | secreted | | |
| C6 domain | 4 | 4 | 4 | 4 |
| (Pfam) | | | | |
| 25-59 | | | | |
| 61-103 | | | | |
| 104-143 | | | | |
| 145-186 | | | | |
| Disulfide bonds | all | all | all | all |
| 26-37 | | | | |
| 38-51 | | | | |
| 41-59 | | | | |
| 62-77 | | | | |
| 83-103 | | | | |
| 105-119 | | | | |
| 111-116 | | | | |
| 125-143 | | | | |
| EGF_2 | + | + | + | + |
| 103-116 | | | | |
| WT N-linked | + | + | + | + |
| Glycosylation | + | + | + | + |
| (153-6, 180-3) | | | | |

Domains and pattern were analyzed according to INTERPRO
Disulfide bonds are according to Swiss-Prot (predicted according to TNFR)
Glycosylation sites were predicted using ProScan.
GRAVY is a hydrophobicity parameter.
TMpred is a predicted transmembrane domain
C6 domain is a TNFR/NGFR domain as deduced by Pfam.
Sp is signal peptide (available from SwissProt database)
EGF_2 is a type of domain
All numbers (for locations of domains etc) are according to WT residues

1. INTERPRO—InterPro is a database of protein families, domains and functional sites, including identified features. These features, which are known to occur in particular (previously identified) proteins, can be applied to unknown protein sequences by using this data. The tool can be found at: www.ebi.ac.uk/interpro/. Further description of Interpro can be found in Mulder et al., (2003) Nucleic Acids Res. 31, 315-318.

2. Swiss-Prot is the well known protein database, which includes information about disulfide bonds for known proteins (see Bairoch et al., (2004) Brief. Bioinform. 5:39-55). In this case, the information about these bonds was taken from the database for the wildtype (WT) protein. The variant proteins were then checked to see if they had the same residues, and hence the same bonds, which they do because the protein sequences are identical through residue 187. Prediction of disulfide bonds was made by using TNFR (tumor necrosis factor receptor) as CD40 protein is also called “Tumor necrosis factor receptor superfamily member 5.” This database also contains such information as the identity of the signal peptide for the wild type CD40 (SwissProt accession number P25942), which is identical for the NJ variants. SwissProt also contains domain predictions.

3. Glycosylation sites were predicted by using ProScan (software that performs a ProSite Scan. ProSite is a database which can be used to identify protein features and also related proteins to a sequence. See Hulo et al., (2004) Nucl. Acids. Res. 32:D134-D137.

4. GRAVY is a hydrophobicity parameter. See Kyte & Doolittle, (1982) J Mol Biol 157:105-132.

TABLE 2

| | WT | NJ1 | NJ2 | NJ3 |
|---|---|---|---|---|
| Ligand binding | + | + | + | + |
| Amino acids - | | | | |
| are they | | | | |
| present? | | | | |
| E74 Y82 N86 | | | | |
| D84 E114 E117 | | | | |
| Note that | | | | |
| numbers are | | | | |
| according to | | | | |
| the WT | | | | |
| sequence | | | | |

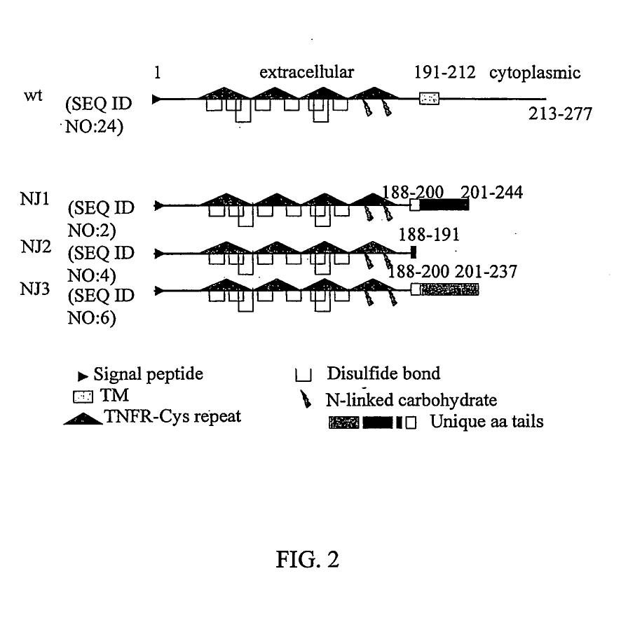

As shown in FIG. 2, NJ1, NJ2, and NJ3 are predicted to encode protein products that have a signal peptide but lack a transmembrane domain, and are thus expected to be secreted from the cell.

A comparison of the tails of NJ1 and NJ3 (for which experimental data is provided) is presented below, with identical portions of the two sequences underlined. All three variants share the same part of the WT sequence (1-187). It should be noted that NJ2 has a very short tail (4 amino acids). The N-termini of NJ1 and NJ3 start identically, followed by divergence between the tail sequences. The invention thus includes a protein with a sequence at least about 90% but preferably at least about 95% homology to residues 188-200 of either NJ1 or NJ3 amino acid sequence, followed by a section that has at least 70% homology from residues 201-end of the sequence.

| | |
|---|---|
| NJ3- | |
| 1. ESWTMGPGESLGRSPGSAESPGGDPHHLRDPVC | (SEQ ID NO:13) |
| HPLGAGLYQKGGQEANQ | |
| 2. ESWTMGPGESLGRWELKGEMRHTGTLDGKKGRG | (SEQ ID NO:11) |
| GSLGVWYHSSATYLGSLGKSLPLS | |
| --NJ1 | |

The amino acids starting position 13 of the unique tail (RSPGSAESPGGDPHHLRDPVCHPLGAGLYQKGGQEANQ) of NJ3 are identical to the entire unique tail of CD40 skipping 6 variant, whose sequence is provided below. The skipping 6 variant is used as a control in the FACS experiments (see Examples, below) and has been shown to have CD40 antagonist activity. See, e.g., U.S. Pat. No. 6,720,182.

The terms “skip6” and “skipping 6”, both of which are used in the application, refer to the same sequence. The tail sequence is underlined. Activity is retained by those NJ variants having the initial 13 amino acid motif in their tails, followed by at least 37 amino acids (for a total length of 50 amino acids); however, the preferred minimum length of tail for these variants seems to be at least 38 amino acids long.

Skipping 6 variant:

>gi|13016698|emb|AJ300189.1|HSA300189 Homo sapiens mRNA for CD40 type II isoform (CD40 gene)

| | |
|---|---|
| M V R L P L Q C V L W G C L L T A V | (SEQ ID NO:??) |
| H P E P P T A C R E K Q Y L I N S Q | |
| C C S L C Q P G Q K L V S D C T E F | |
| T E T E C L P C G E S E F L DT W N | |
| R E T H C H Q H K Y C D P N L G L R | |
| V Q Q K G T S E T D T I C T C E E G | |
| W H C T S E A C E S C V L H R S C S | |
| P G F G V K Q I A T G VS D T I C E | |
| P C P V G F F S N V S S A F E K C H | |
| P W T R S P G S A E S P G G D P H H | |
| L R D P V C H P L G A G L Y Q K G G | |
| Q E A N Q | |

The expression of different CD40 splice variants was checked in total RNA derived from bone marrow (Clontech, Cat #110932), spleen (Ambion, Cat # 111PO106B), thymus (Clontech, Cat #1070319) and colon (Ichilov Hos., Cat # CG335), as well as the cell lines K562 (chronic myelogenous leukemia cell line; ATCC: CCL-243) and NL564 (EBV transformed human normal lymphoblasts).

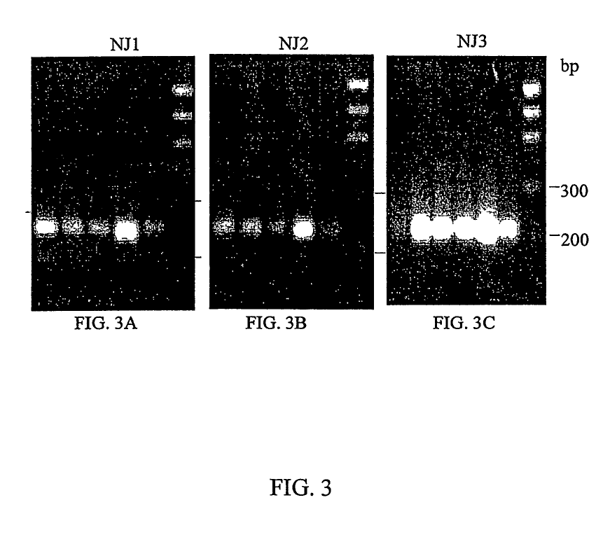

Total RNA was extracted using Tri-reagent (MRC), and removal of DNA contaminates was carried out by RNasy mini kit (QIAGEN). Ten μg was used in 250 μl RT reaction, with reverse transcriptase (Superscript II; Promega) and oligo-dT, according to the manufacturer's instructions. The resulting cDNA was used as a template for PCR using specific primers, with the following cycling conditions: 94° C.-15 min, 36 cycles of: 94° C.-1 min, 63° C.-1 min, 72° C.-1 min, and a final extension of 72° C.-10 min. The results presented in FIG. 3 show that the three splice variants were indeed detected.

Some aspects of the invention relate to CD40 splice variant proteins, nucleic acid molecules encoding the same, recombinant vectors and host cells comprising such nucleic acid molecules, antibodies which bind to CD40 splice variant proteins and hybridomas which produce such proteins. Some aspects of the invention relate to assays, reagents and kits for detecting the presence of CD40 splice variant protein or transcript in samples. Some aspects of the invention relate to methods and compositions for modulating CD40-CD40 ligand interactions and for treating individuals with diseases. The present invention also relates to pharmaceutical compositions that are suitable for the treatment of diseases and pathological conditions, which can be ameliorated or cured by decreasing the levels of any one of the ligands of the original CD40. The term “ligands” is meant to refer to not only CD 154, but to any other compounds, e.g., TRAF3 or TRAF2, which are known to interact with CD40.

CD40 Splice Variant Protein

The present invention provides substantially purified CD40 splice variants, including substantially purified human CD40 splice variants, functionally active fragments thereof that comprise a unique tail sequence, substantially purified murine CD40 splice variants and functionally active fragments thereof that comprise a unique tail sequence. Substantially purified human CD40 splice variants include those selected from the group consisting of proteins having amino acid sequences consisting of: SEQ ID NO:2, SEQ ID NO:4, SEQ ID NO:6, SEQ ID NO:7 and SEQ ID NO:8. Substantially purified murine CD40 splice variants include those selected from the group consisting of proteins having amino acid sequences consisting of: SEQ ID NO:9 and SEQ ID NO:10. Unique tail sequences of human CD40 splice variants are set forth in SEQ ID NO:11, SEQ ID NO:12, SEQ ID NO:13, SEQ ID NO:14 and SEQ ID NO:15. Unique tail sequences of murine CD40 splice variants are selected are set forth in SEQ ID NO:16 and SEQ ID NO:17. In addition, CD40 splice variant proteins include fragments, homologues and fragments of homologues of the proteins including fragments, homologues and fragments of homologues of the unique tails.

The human unique tail region of SEQ ID NO:2 includes the 57 amino acids at the C terminus of SEQ ID NO:2. The coding sequences of the human unique tail region of SEQ ID NO:2 is the nucleotide sequence in SEQ ID NO:1 that encodes the 57 amino acids at the C terminus of SEQ ID NO:2. The human unique tail sequence is SEQ ID NO:11.

The human unique tail region of SEQ ID NO:4 includes the 4 amino acids at the C terminus of SEQ ID NO:4. The coding sequences of the human unique tail region of SEQ ID NO:4 is the nucleotide sequence in SEQ ID NO:3 that encodes the 4 amino acids at the C terminus of SEQ ID NO:4. The human unique tail sequence is SEQ ID NO:12.

The human unique tail region of SEQ ID NO:6 includes the 50 amino acids at the C terminus of SEQ ID NO:6. The coding sequences of the human unique tail region of SEQ ID NO:6 is the nucleotide sequence in SEQ ID NO:13. The human unique tail sequence is SEQ ID NO:14 includes the 21 amino acids at the C terminus of SEQ ID NO:7.

The human unique tail region of SEQ ID NO:7 includes the 21 amino acids at the C terminus of SEQ ID NO:7. The human unique tail sequence is SEQ ID NO:14.

The human unique tail region of SEQ ID NO:8 includes the 42 amino acids at the C terminus of SEQ ID NO:8. The human unique tail sequence is SEQ ID NO:15.

The murine unique tail region of SEQ ID NO:9 includes the 42 amino acids at the C terminus of SEQ ID NO:9. The murine unique tail sequence is SEQ ID NO:16.

The unique tail region of SEQ ID NO:10 includes the 25 amino acids at the C terminus of SEQ ID NO:10. The murine unique tail sequence is SEQ ID NO:17.

Aspects of the present invention provide a protein or polypeptide comprising or consisting of an amino acid sequence, termed herein “CD40 variant”, having the sequence as depicted in any one of SEQ ID NO:2, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16 and SEQ ID NO:17 and fragments and homologues thereof, preferably having a length of at least 10 amino acids. Homologues comprise the above amino acid sequences in which one or more of the amino acid residues has been substituted (by conservative or non-conservative substitution) added, deleted, or chemically modified. The sequence variations of the homologues are preferably those that are considered conserved substitutions. Thus, for example, a protein with a sequence having at least 90% sequence identity with the products identified as SEQ ID NO:2, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16 and SEQ ID NO:17 preferably by utilizing conserved substitutions. The CD40 splice variants may be (i) one in which one or more of the amino acid residues in a sequence listed above are substituted with a conserved or non-conserved amino acid residue (preferably a conserved amino acid residue), or (ii) one in which one or more of the amino acid residues includes a substituent group, or (iii) one in which the CD40 splice variant is fused with another compound, such as a compound increase the half-life of the protein (for example, polyethylene glycol (PEG)), or a moiety which serves as targeting means to direct the protein to its target tissue or target cell population (such as an antibody), or (iv) one in which additional amino acids are fused to the CD40 splice variant. Such fragments, variants and derivatives are deemed to be within the scope of those skilled in the art from the teachings herein.

Substantially purified human CD40 splice variants and substantially purified murine CD40 splice variants can be isolated from natural sources, produced by recombinant DNA methods or synthesized by standard protein synthesis techniques. Substantially purified functionally active fragments of human CD40 splice variants that comprise at least ten amino acid residues, including four amino acid residues of a unique tail sequence and substantially purified functionally active fragments of murine CD40 splice variants that comprise at least ten amino acid residues including five amino acid residues of a unique tail sequence can be produced by processing protein isolated from natural sources, produced by recombinant DNA methods or synthesized by standard protein synthesis techniques.

CD40 variants of the invention that retain the ligand-binding (extracellular) domain of the original CD40 are capable of binding to its ligands (for example the CD154) and decreasing in the individual the amounts of such free ligands available for binding to the original CD40. Thus, CD40 variants of the invention may act as “scavengers” of CD154, since they can bind those ligands without causing signal transduction as a result of the binding, which effectively lowers the amount of the ligands. Since the variants are secreted they can exert their scavenging effect even in body fluids.

CD40 splice variants of the invention are soluble and bind to CD154. Thus, they compete with CD40 on cells associated with the immune system. The presence of the CD40 splice variant reduces the signaling activity that occurs when CD40+ cells interact with CD154+ cells. The soluble alternatively spliced CD40 thus modulates immune activity.

Accordingly, the CD40 splice variants may be used as a pharmaceutical to modulate immune activity, particularly the immune activity associated with CD40-CD154 interaction as well as antigens against which antibodies my be raised.

Antibodies

Some embodiments of the present invention provide anti-CD40 splice variant antibodies; that is antibodies directed against the CD40 splice variants. The antibodies specifically bind to a CD40 variant, particularly at epitopes that include amino acid residues of the unique tail. The antibodies are useful in protein purification assays as well as for both for diagnostic and therapeutic purposes.

Antibodies according to the present invention preferably comprise those which specifically interact with the polypeptides of the present invention and not with wild type CD40 protein or other isoforms thereof. Such antibodies are directed, for example, to the unique sequence portions of the polypeptide variants of the present invention (e.g., the previously described unique tails of the NJ variants) or to unique sequences, which bridge the CD40 common portion and the unique sequence regions (the previously described bridge portions). The antibody is capable of distinguishing the CD40 variant protein from the wild type CD40 protein corresponding to SwissProt accession number P25942.

Preferably, the antibody of this aspect of the present invention specifically binds at least one epitope of the polypeptide variants of the present invention. As used herein, the term “epitope” refers to any antigenic determinant on an antigen to which the paratope of an antibody binds.

Antibodies of the invention specifically bind to an epitope on a particular unique tail region of a human CD40 splice variant or an epitope on a particular unique tail region of a murine CD40 splice variant. The present invention relates to antibodies that bind to an epitope that is present on a unique tail sequence of human CD40 or a unique tail sequence of murine CD40. In some embodiments, the antibodies specifically bind to epitopes that comprise at least 4 amino acid residues of a unique tail sequence.

Antibodies may be used to purify the human CD40 splice variant protein or murine CD40 splice variant protein from natural sources or recombinant expression systems using well known techniques such as affinity chromatography. Antibodies are useful to detect the presence of such protein in a sample and to determine if cells are expressing the protein. Moreover, antibodies are useful as therapeutics in methods of modulating CD40-CD40 ligand interactions.

The present invention further encompasses an antibody or an antibody fragment that binds specifically to an amino acid sequence (epitope) present in any of the amino acid sequences below:

According to one aspect of the present invention there is provided an isolated polypeptide encoding for the CD40 new variant NJ1 (SEQ ID NO: 2), comprising a first amino acid sequence being at least 90% homologous to amino acids 1-187 of wild type CD40 corresponding to GI:15214587 (SEQ ID NO: 24), and a second amino acid sequence being at least 70%, optionally at least 80%, preferably at least 85%, more preferably at least 90% and most preferably at least 95% homologous to a polypeptide having the sequence ESWTMGPGESLGRWELKGEMRHTGTLDGKKGRGGSLGVWYHSSATYLGSLGKSLPLS (SEQ ID NO:11), wherein said first and said second amino acid sequences are contiguous and in a sequential order.

According to another aspect of the present invention there is provided an isolated polypeptide encoding for a tail of variant NJ1, comprising a polypeptide having the sequence being at least 70%, optionally at least 80%, preferably at least 85%, more preferably at least 90% and most preferably at least 95% homologous to a polypeptide having the sequence

| | |
|---|---|
| ESWTMGPGESLGRWELKGEMRHTGTLDGKKGRGGSL | (SEQ ID NO:11) |
| GVWYHSSATYLGSLGKSLPLS. | |

According to another aspect of the present invention there is provided a bridge portion of SEQ ID NO:2, including a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise GE, having a structure as follows (numbering according to SEQ ID NO:2): a sequence starting from any of amino acid numbers (187−x) to 187; ending at any of amino acid numbers 188+((n−2)−x), in which x varies from 0 to (n−2).

For example, for peptides of 10 amino acids (such that n=10), the starting position could be as “early” in the sequence as amino acid number 179 if x=n−2=8 (i.e., 179=187−8), such that the peptide would end at amino acid number 188 (188+(8−8=0)). On the other hand, the peptide could start at amino acid number 187 if x=0 (i.e., 187=187−0), and could end at amino acid 196 (188+(8−0=8)).

The bridge portion above may optionally comprise a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: VCGE (SEQ ID NO:38), CGES (SEQ ID NO:39), GESW (SEQ ID NO:40). All peptides feature GE as a portion thereof. Peptides of from about five to about nine amino acids could optionally be similarly constructed.

According to another aspect of the present invention there is provided an isolated polypeptide encoding for the CD40 new variant NJ2 (SEQ ID NO: 4), including a first amino acid sequence being at least 90% homologous to amino acids 1-187 of wild type CD40 corresponding to GI:15214587 (SEQ ID NO: 24), and a second amino acid sequence being at least 50%, and preferably at least 75% homologous to a polypeptide having the sequence LGLE (SEQ ID NO:12), wherein said first and said second amino acid sequences are contiguous and in a sequential order.

According to another aspect of the present invention there is provided an isolated polypeptide encoding for a tail of variant NJ2, comprising a polypeptide having the sequence being at least 50% and preferably at least 75%, and most preferably at least 100% homologous to a polypeptide having the sequence LGLE (SEQ ID NO:12).

According to another aspect of the present invention there is provided a bridge portion of SEQ ID NO:4, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise GL, having a structure as follows (numbering according to SEQ ID NO:4): a sequence starting from any of amino acid numbers 187−x to 187; ending at any of amino acid numbers 188+((n−2)−x), in which x varies from 0 to n−2, such that the number of the ending amino acid is not larger than 191.

For example, for peptides of 10 amino acids (such that n=10), the starting position could be as “early” in the sequence as amino acid number 179 if x=n−2=8 (i.e., 179=187−8), such that the peptide would end at amino acid number 188 (188+(8−8=0)). On the other hand, the peptide could start at amino acid number 182 if x=5 (i.e., 182=187−5), and could end at amino acid 191 (188+(8−5=3)). Since the last amino acid number is 191, the number of the ending amino acid of the bridge portion cannot be larger than 191.

The bridge portion above which comprises a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: VCGL (SEQ ID NO:41), CGLG (SEQ ID NO:42), GLGL (SEQ ID NO:43). All peptides feature GL as a portion thereof. Peptides of from about five to about nine amino acids could optionally be similarly constructed.

According to another aspect of the present invention there is provided an isolated polypeptide encoding for the CD40 new variant NJ3 (SEQ ID NO: 6), comprising a first amino acid sequence being at least 90% homologous to amino acids 1-187 of wild type CD40 corresponding to GI:15214587 (SEQ ID NO: 24), and a second amino acid sequence being at least 70%, optionally at least 80%, preferably at least 85%, more preferably at least 90% and most preferably at least 95% homologous to a polypeptide having the sequence ESWTMGPGESLGRSPGSAESPGGDPHHLRDPVCHPLGAGLYQKGGQEANQ (SEQ ID NO:13), wherein said first and said second amino acid sequences are contiguous and in a sequential order.

According to another aspect of the present invention there is provided an isolated polypeptide encoding for a tail of variant NJ3, comprising a polypeptide having the sequence being at least 70%, optionally at least 80%, preferably at least 85%, more preferably at least 90% and most preferably at least 95% homologous to a polypeptide having the sequence

| | |
|---|---|
| ESWTMGPGESLGRSPGSAESPGGDPHHLRDPVCHPL | (SEQ ID NO:13) |
| GAGL YQKGGQEANQ. | |

According to another aspect of the present invention there is provided a bridge portion of SEQ ID NO:6, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise GG, having a structure as follows (numbering according to SEQ ID NO:6): a sequence starting from any of amino acid numbers 187−x to 187; ending at any of amino acid numbers 188+((n−2)−x), in which x varies from 0 to n−2, such that the number of the ending amino acid is not larger than 237.

For example, for peptides of 10 amino acids (such that n=10), the starting position could be as “early” in the sequence as amino acid number 179 if x=n−2=8 (i.e., 179=187−8), such that the peptide would end at amino acid number 188 (188+(8−8=0)). On the other hand, the peptide could start at amino acid number 187 if x=0 (i.e., 187=187−0), and could end at amino acid 196 (188+(8−0=8)).

The bridge portion above may optionally comprise a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: VCGE (SEQ ID NO:38), CGES (SEQ ID NO:39), GESW (SEQ ID NO:40). All peptides feature GE as a portion thereof. Peptides of from about five to about nine amino acids could optionally be similarly constructed.

Epitopic determinants usually consist of chemically active surface groupings of molecules such as amino acids or carbohydrate side chains and usually have specific three dimensional structural characteristics, as well as specific charge characteristics.

The production of antibodies and the protein structures of complete, intact antibodies, Fab fragments and F(ab)₂ fragments and the organization of the genetic sequences that encode such molecules are well known and are described, for example, in Harlow, E. and D. Lane (1988) ANTIBODIES: A Laboratory Manual, Cold Spring Harbor Laboratory, Cold Spring Harbor, N.Y. which is incorporated herein by reference. Briefly, for example, a CD40 splice variant protein, or an immunogenic fragment thereof is injected into mice. The spleen of the mouse is removed, the spleen cells are isolated and fused with immortalized mouse cells. The hybrid cells, or hybridomas, are cultured and those cells that secrete antibodies are selected. The antibodies are analyzed and, if found to specifically bind to the CD40 splice variant protein, the hybridoma which produces them is cultured to produce a continuous supply of antibodies.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See, e.g., Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. Biochem. J. 73: 119-126 (1959). Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al., Proc. Nat'l Acad. Sci. USA 69:2659-62 (1972). Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow & Filpula, Methods 2: 97-105 (1991); Bird et al., Science 242:423-426 (1988); Pack et al., Bio/Technology 11: 1271-77 (1993); and U.S. Pat. No. 4,946,778, which are hereby incorporated by reference in their entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, e.g., Larrick & Fry Methods, 2: 106-10 (1991).

Humanized forms of non-human, e.g., murine antibodies are chimeric molecules of immunoglobulins, immunoglobulin chains or fragments thereof (such as Fv, Fab, Fab′, F(ab′).sub.2 or other antigen-binding subsequences of antibodies), which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. Jones et al., Nature, 321:522-525 (1986); Riechmann et al., Nature, 332:323-329 (1988); and Presta, Curr. Op. Struct. Biol., 2:593-596 (1992).

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers Jones et al., Nature, 321:522-525 (1986); Riechmann et al., Nature 332:323-327 (1988); Verhoeyen et al., Science, 239:1534-1536 (1988), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1):86-95 (1991). Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

The diagnostic reagents described hereinabove can also be included in kits. For example a kit for diagnosing predisposition to, or presence of CD40-related disease in a subject can include an antibody directed at the unique amino acid sequence of one or more of variants NJ1-3 in a one container and a solid phase for attaching multiple biological samples packaged in a second container with appropriate buffers and preservatives and used for diagnosis.

Nucleic Acid Molecules

Some aspects of the present invention relate to novel isolated nucleic acid molecules that encode proteins comprising or consisting of the sequence of SEQ ID NO:2, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16 and SEQ ID NO:17 and fragments and homologues thereof. Some aspects of the present invention relate to novel isolated nucleic acid molecules comprising or consisting of the sequence of any of one of SEQ ID NO:1, SEQ ID NO:3, SEQ ID NO:5, and fragments of said coding sequence having at least 20 nucleic acids and/or comprising a sequence having at least 90% identity to SEQ ID NO:1, SEQ ID NO:3, or SEQ ID NO:5. The present invention further provides nucleic acid molecule comprising or consisting of a sequence which encodes the above amino acid sequences, (including the fragments and homologues of the amino acid sequences). In some embodiments, the present invention relates to an isolated nucleic acid molecule that comprises a nucleotide sequence that encodes a CD40 splice variants selected from the group consisting of SEQ ID NO:2, SEQ ID NO:4, SEQ ID NO:6, SEQ ID NO:7, SEQ ID NO:8, SEQ ID NO:9 and SEQ ID NO:10. In some embodiments, the nucleic acid molecules consist of a nucleotide sequence that encodes SEQ ID NO:2, SEQ ID NO:4, SEQ ID NO:6, SEQ ID NO:7, SEQ ID NO:8, SEQ ID NO:9 and SEQ ID NO:10. In some embodiments, the nucleic acid molecules comprise the coding sequence in SEQ ID NO:1, SEQ ID NO:3, or SEQ ID NO:5. In some embodiments, the nucleic acid molecules consist of the nucleotide sequence set forth in SEQ ID NO:1, SEQ ID NO:3, or SEQ ID NO:5. The isolated nucleic acid molecules of the invention are useful to prepare constructs and recombinant expression systems for preparing CD40 splice variants of the invention. Due to the degenerative nature of the genetic code, a plurality of alternative nucleic acid sequences, beyond those depicted by any one of SEQ ID NO:1, SEQ ID NO:3, or SEQ ID NO:5, can code for the amino acid sequence of the invention. Nucleic acid sequence which code for the same amino acid sequences depicted any one of the sequences SEQ ID NO:2, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16 and SEQ ID NO:17 are also aspects of the present invention. In addition, the present invention further provides expression vectors and cloning vectors comprising any of the above nucleic acid sequences, as well as host cells transfected by said vectors.

The present invention relates to isolated nucleic acid molecules that comprise a nucleotide sequence identical or complementary to a fragment of a nucleic acid molecules which encodes any one of the sequences SEQ ID NO:2, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16 and SEQ ID NO:17 or a fragment or homologue thereof. Preferably, the isolated nucleic acid molecules comprise at least 10 nucleotides, in some embodiments 15-150 nucleotides and in some embodiments preferably 15-30 nucleotides. In some embodiments, the nucleic acid molecules comprise 16 or more nucleotides, preferably 24 nucleotides.

The present invention relates to isolated nucleic acid molecules that comprise a nucleotide sequence identical or complementary to a fragment of SEQ ID NO:1, SEQ ID NO:3, or SEQ ID NO:5 which is at least 10 nucleotides. In some embodiments, the isolated nucleic acid molecules consist of a nucleotide sequence identical or complementary to a fragment of SEQ ID NO:1, SEQ ID NO:3, or SEQ ID NO:5 which is at least 10 nucleotides. In some embodiments, the isolated nucleic acid molecules comprise or consist of a nucleotide sequence identical or complementary to a fragment of SEQ ID NO:1, SEQ ID NO:3, or SEQ ID NO:5 which is 15-150 nucleotides. In some embodiments, the isolated nucleic acid molecules comprise or consist of a nucleotide sequence identical or complementary to a fragment of SEQ ID NO:1, SEQ ID NO:3, or SEQ ID NO:5 which is 15-30 nucleotides. Isolated nucleic acid molecules that comprise or consist of a nucleotide sequence identical or complementary to a fragment of SEQ ID NO:1, SEQ ID NO:3, or SEQ ID NO:5 which is at least 10 nucleotides are useful as probes for identifying genes and cDNA sequence having SEQ ID NO:1, SEQ ID NO:3, or SEQ ID NO:5, respectively, PCR primers for amplifying genes and cDNA having SEQ ID NO:1, SEQ ID NO:3, or SEQ ID NO:5, respectively, and antisense molecules for inhibiting transcription and translation of genes and cDNA, respectively, which encode CD40 splice variants having the amino acid sequence of SEQ ID NO:2, SEQ ID NO:4, SEQ ID NO:6, SEQ ID NO:7, SEQ ID NO:8, SEQ ID NO:9 or SEQ ID NO:10.

The present invention includes labeled oligonucleotides that are useful as probes for performing oligonucleotide hybridization methods to identify CD40 splice variants. Accordingly, the present invention includes probes that can be labeled and hybridized to unique nucleotide sequences of CD40 splice variants. The labeled probes of the present invention are labeled with radiolabeled nucleotides or are otherwise detectable by readily available nonradioactive detection systems. In some preferred embodiments, probes comprise oligonucleotides consisting of between 10 and 100 nucleotides. In some preferred embodiments, probes comprise oligonucleotides consisting of between 10 and 50 nucleotides. In other preferred embodiments, probes comprise oligonucleotides consisting of between 12 and 20 nucleotides. The probes preferably contain nucleotide sequence completely identical or complementary to a fragment of a nucleotide sequences that encode the unique tails of the CD40 splice variants.

Using standard techniques and readily available starting materials, a nucleic acid molecule that encodes each of the CD40 splice variants of the invention may be isolated from a cDNA library, using probes or primers which are designed using the nucleotide sequence information disclosed herein with particular reference to the coding sequence of the unique tail. A cDNA library may be generated by well known techniques. A cDNA clone that contains one of the nucleotide sequences set out is identified using probes that comprise at least a portion of the nucleotide sequence disclosed in SEQ ID NO:1, SEQ ID NO:3, or SEQ ID NO:5. The probes have at least 10 nucleotides, preferably 16 nucleotides, and preferably 24 nucleotides. The probes are used to screen the cDNA library using standard hybridization techniques. Alternatively, genomic clones may be isolated using genomic DNA from any human cell as a starting material.

The cDNA that encodes a CD40 splice variant may be used as a molecular marker in electrophoresis assays in which cDNA from a sample is separated on an electrophoresis gel and CD40 splice variant probes are used to identify bands which hybridize to such probes. Specifically, SEQ ID NO:1 or portions thereof, SEQ ID NO:3 or portions thereof, and SEQ ID NO:5 or portions thereof, may be used as a molecular marker in electrophoresis assays in which cDNA from a sample is separated on an electrophoresis gel and CD40 splice variant specific probes are used to identify bands which hybridize to them, indicating that the band has a nucleotide sequence complementary to the sequence of the probes. The isolated nucleic acid molecule provided as a size marker will show up as a positive band which is known to hybridize to the probes and thus can be used as a reference point to the size of cDNA that encodes a particular CD40 splice variant. Electrophoresis gels useful in such an assay include standard polyacrylamide gels as described in Sambrook et al., Molecular Cloning a Laboratory Manual, Second Ed. Cold Spring Harbor Press (1989) which is incorporated herein by reference.

The nucleotide sequences that encode splice variant proteins may be used to design probes, primers and complimentary molecules, which specifically hybridize to the nucleotide sequences that encode the unique tails of the CD40 splice variants. For example, the nucleotide sequences in SEQ ID NO:1, SEQ ID NO:3, and SEQ ID NO:5 may be used to design probes, primers and complimentary molecules that specifically hybridize to the nucleotide sequences that encode the unique tails. Probes, primers and complimentary molecules that specifically hybridize to nucleotide sequence that the unique tails of the CD40 splice variants may be designed routinely by those having ordinary skill in the art.

Nucleic acid molecules that encode the CD40 splice variants may be used as part of pharmaceutical compositions for gene therapy and antisense therapy.

In some embodiments, the CD40 splice variant is provided in accordance with the present invention by expression of such polypeptides in vivo, which is often referred to as gene therapy. The expression of CD40 splice variants may be increased by providing to an individual a genetic construct which comprises coding sequences for coding for the CD40 splice variants under the control of suitable control elements ending its expression in the desired host. The nucleic acid sequences of the invention may be employed in combination with a suitable pharmaceutical carrier. Such compositions comprise a therapeutically effective amount of the compound, and a pharmaceutically acceptable carrier or excipient. Such a carrier includes but is not limited to saline, buffered saline, dextrose, water, glycerol, ethanol, and combination thereof. The formulation should suit the mode of administration.

Cells from a patient may be engineered with a nucleic acid sequence (DNA or RNA) encoding a polypeptide ex vivo, with the engineered cells then being provided to a patient to be treated with the polypeptide. Such methods are well-known in the art. For example, cells may be engineered by procedures known in the art by use of a retroviral particle containing RNA encoding a polypeptide of the present invention.

Similarly, cells may be engineered in vivo for expression of a polypeptide in vivo by procedures known in the art. As known in the art, a producer cell for producing a retroviral particle containing RNA encoding the polypeptides of the present invention may be administered to a patient for engineering cells in vivo and expression of the polypeptide in vivo. These and other methods for administering products of the present invention by such method should be apparent to those skilled in the art from the teachings of the present invention.

Nucleic acid molecules that encode a CD40 splice variant protein may be delivered using any one of a variety of delivery components, such as recombinant viral expression vectors or other suitable delivery means, so as to affect their introduction and expression in compatible host cells. In general, viral vectors may be DNA viruses such as recombinant adenoviruses and recombinant vaccinia viruses or RNA viruses such as recombinant retroviruses. Other recombinant vectors include recombinant prokaryotes that can infect cells and express recombinant genes. In addition to recombinant vectors, other delivery components are also contemplated such as encapsulation in liposomes, transferrin-mediated transfection and other receptor-mediated means. The invention is intended to include such other forms of expression vectors and other suitable delivery means which serve equivalent functions and which become known in the art subsequently hereto.

In one embodiment of the present invention, DNA is delivered to competent host cells by means of an adenovirus. One skilled in the art would readily understand this technique of delivering DNA to a host cell by such means. Although the invention preferably includes adenovirus, the invention is intended to include any virus which serves equivalent functions.

In another embodiment of the present invention, RNA is delivered to competent host cells by means of a retrovirus. One skilled in the art would readily understand this technique of delivering RNA to a host cell by such means. Any retrovirus that serves to express the protein encoded by the RNA is intended to be included in the present invention.

Retroviruses from which the retroviral plasmid vectors mentioned above may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myelproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, psi-2, psi-AM, PA12, T19-14X, VT-19-17-H2, psi-CRE, psi-CRIP, GP+E-86, GP+envAm12, and DAN cell lines as described in Miller (Human Gene Therapy, Vol. 1, pg. 5-14, (1990)). The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles that include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

The genes introduced into cells may be placed under the control of inducible promoters, such as the radiation-inducible Egr-1 promoter, (Maceri, H. J., et al., Cancer Res., 56(19):4311 (1996), to stimulate variant production or antisense inhibition in response to radiation, e.g., radiation therapy for treating tumors.

In another embodiment of the present invention, nucleic acid is delivered through folate receptor means. The nucleic acid sequence to be delivered to a cell is linked to polylysine and the complex is delivered to cells by means of the folate receptor. U.S. Pat. No. 5,108,921 issued Apr. 28, 1992 to Low et al., which is incorporated herein by reference, describes such delivery components.

According to one aspect of the invention, expression of CD40 splice variants may be modulated through antisense technology, which controls gene expression through hybridization of complementary nucleic acid sequences, i.e., antisense DNA or RNA, to the control, 5′ or regulatory regions of the gene encoding variant product. Nucleic acid molecules comprising or consisting of a non-coding sequence which is complementary to that of a CD40 splice variant transcript or complementary to a sequence having at least 90% identity to said sequences or a fragment of said sequences are provided. The complementary sequence may be a DNA sequence which hybridizes with the sequences of CD40 splice variant transcript or hybridizes to a portion of those sequences having a length sufficient to inhibit the transcription of the complementary sequences. The complementary sequence may be a DNA sequence which can be transcribed into an mRNA being an antisense to the mRNA transcribed from the CD40 splice variant transcript or into an mRNA which is an antisense to a fragment of the mRNA transcribed from the CD40 splice variant transcript which has a length sufficient to hybridize with the mRNA transcribed from any one of the CD40 splice variant transcripts, so as to inhibit its translation. The complementary sequence may also be the mRNA or the fragment of the mRNA itself. In some embodiments, the 5′ coding portion of the nucleic acid sequence that codes for the product of the present invention is used to design an antisense oligonucleotide of from about 10 to 40 base pairs in length. Oligonucleotides derived from the transcription start site, e.g., between position −10 and +10 from the start site, are preferred. An antisense DNA oligonucleotide is designed to be complementary to a region of the nucleic acid sequence involved in transcription (Lee et al., Nucl. Acids, Res., 6:3073, (1979); Cooney et al., Science 241:456, (1988); and Dervan et al. Science 251:1360, (1991)), thereby preventing transcription and the production of the variant products. An antisense RNA oligonucleotide hybridizes to the mRNA in vivo and blocks translation of the mRNA molecule into the variant products (Okano, Neurochem. 56:560, (1991)). The antisense constructs can be delivered to cells by procedures known in the art such that the antisense RNA or DNA may be expressed in vivo. The antisense may be antisense mRNA or DNA sequence capable of coding such antisense mRNA. The antisense mRNA or the DNA coding thereof can be complementary to the full sequence of nucleic acid sequences coding for the CD40 splice variant protein or to a fragment of such a sequence which is sufficient to inhibit production of a protein product. Antisense technologies can also be used for inhibiting expression of one variant as compared to the other, or inhibiting the expression of the variant/s as compared to the original sequence. Nucleic acid molecules that encode the CD40 splice variants may be used in expression systems to make CD40 splice variant proteins.

Methods of Using Nucleic Acid Molecules to Make Protein

One having ordinary skill in the art can isolate the nucleic acid molecule that encode a CD40 splice variant and insert it into an expression vector using standard techniques and readily available starting materials. The present invention relates to a recombinant expression vector that comprises a nucleotide sequence that encodes a CD40 splice variant that comprises the amino acid sequence of SEQ ID NO:2, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16 or SEQ ID NO:17. In some embodiments, the recombinant expression vector comprises the nucleotide sequence set forth in SEQ ID NO:1, SEQ ID NO:3 or SEQ ID NO:5.

The recombinant expression vectors of the invention are useful for transforming hosts to prepare recombinant expression systems for preparing the CD40 variants of the invention. As will be understood by those of skill in the art, it may be advantageous to produce CD40 variants product-encoding nucleotide sequences possessing codons other than those which appear in SEQ ID NO:1, SEQ ID NO:3 or SEQ ID NO: 5 which are those which naturally occur in the human genome. Codons preferred by a particular prokaryotic or eukaryotic host (Murray et al., Nuc Acids Res., 17:477-508, (1989)) can be selected, for example, to increase the rate of variant product expression or to produce recombinant RNA transcripts having desirable properties, such as a longer half-life, than transcripts produced from naturally occurring sequence.

The nucleic acid sequences of the present invention can be engineered in order to alter a CD40 splice variants products coding sequences for a variety of reasons, including but not limited to, alterations that modify the cloning, processing and/or expression of the product. For example, alterations may be introduced using techniques that are well known in the art, e.g., site-directed mutagenesis, to insert new restriction sites, to alter glycosylation patterns, to change codon preference, etc.

The present invention also includes recombinant constructs comprising one or more of the sequences as broadly described above. The constructs comprise a vector, such as a plasmid or viral vector, into which nucleic acid sequences of the invention have been inserted, in a forward or reverse orientation. In a preferred aspect of this embodiment, the constructs further comprise regulatory sequences, including, for example, a promoter, operably linked to the sequence. Large numbers of suitable vectors and promoters are known to those of skill in the art, and are commercially available. Appropriate cloning and expression vectors for use with prokaryotic and eukaryotic hosts are also described in Sambrook et al., supra.

The present invention also relates to host cells which are genetically engineered with vectors of the invention, and the production of the product of the invention by recombinant techniques. Host cells are genetically engineered, i.e., transduced, transformed or transfected, with the vectors of this invention which may be, e.g., a cloning vector or an expression vector. The vector may be, e.g., in the form of a plasmid, a viral particle, a phage, etc. The engineered host cells can be cultured in conventional nutrient media modified as appropriate for activating promoters, selecting transformants or amplifying the expression of the variant nucleic acid sequence. The culture conditions, such as temperature, pH and the like, are those previously used with the host cell selected for expression and will be apparent to those skilled in the art.

The present invention relates to a host cell that comprises the recombinant expression vector that includes a nucleotide sequence that encodes a CD40 variant selected from the group consisting of SEQ ID NO:2, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16 and SEQ ID NO:17 and fragments and homologues thereof. In some embodiments, the present invention relates to a host cell that comprises the recombinant expression vector that includes a nucleotide sequence that comprises SEQ ID NO:1, SEQ ID NO:3 or SEQ ID NO:5. Host cells for use in well known recombinant expression systems for production of proteins are well known and readily available. Examples of host cells include bacteria cells such as E. coli, yeast cells such as S. cerevisiae, insect cells such as S. frugiperda, non-human mammalian tissue culture cells chinese hamster ovary (CHO) cells and human tissue culture cells such as HeLa cells.

The nucleic acid sequences of the present invention may be included in any one of a variety of expression vectors for expressing a product. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host. The appropriate DNA sequence may be inserted into the vector by a variety of procedures. In general, the DNA sequence is inserted into an appropriate restriction endonuclease site(s) by procedures known in the art. Such procedures and related sub-cloning procedures are deemed to be within the scope of those skilled in the art.

The DNA sequence in the expression vector is operatively linked to an appropriate transcription control sequence (promoter) to direct mRNA synthesis. Examples of such promoters include: LTR or SV40 promoter, the E. coli lac or trp promoter, the phage lambda PL promoter, and other promoters known to control expression of genes in prokaryotic or eukaryotic cells or their viruses. The expression vectors also contains a ribosome binding site for translation initiation, and a transcription terminator. The vector may also include appropriate sequences for amplifying expression. In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vectors containing the appropriate DNA sequence as described above, as well as an appropriate promoter or control sequence, may be employed to transform an appropriate host to permit the host to express the protein. Examples of appropriate expression hosts include: bacterial cells, such as E. coli, Streptomyces, Salmonella typhimurium; fungal cells, such as yeast; insect cells such as Drosophila and Spodoptera Sf9; animal cells such as CHO, COS, HEK 293 or Bowes melanoma; adenoviruses; plant cells, etc. The selection of an appropriate host is deemed to be within the scope of those skilled in the art from the teachings herein. The invention is not limited to any particular host cells which can be employed.

One having ordinary skill in the art can use commercial expression vectors and systems or others to produce a CD40 splice variant of the invention using routine techniques and readily available starting materials. Thus, the desired proteins can be prepared in both prokaryotic and eukaryotic systems, resulting in a spectrum of processed forms of the protein. Expression systems containing the requisite control sequences, such as promoters and polyadenylation signals, and preferably enhancers, are readily available and known in the art for a variety of hosts. See, e.g., Sambrook et al., Molecular Cloning a Laboratory Manual, Second Ed. Cold Spring Harbor Press (1989) which is incorporated herein by reference.

A wide variety of eukaryotic hosts are also now available for production of recombinant foreign proteins. As in bacteria, eukaryotic hosts may be transformed with expression systems which produce the desired protein directly, but more commonly signal sequences are provided to effect the secretion of the protein. Eukaryotic systems have the additional advantage that they are able to process introns that may occur in the genomic sequences encoding proteins of higher organisms. Eukaryotic systems also provide a variety of processing mechanisms which result in, for example, glycosylation, carboxy-terminal amidation, oxidation or derivatization of certain amino acid residues, and conformational control.

Commonly used eukaryotic systems include, e.g., yeast, fungal cells, insect cells, mammalian cells, avian cells, and cells of higher plants. Suitable promoters are available that are compatible and operable for use in each of these host types as well as are termination sequences and enhancers, e.g. the baculovirus polyhedron promoter. As above, promoters can be either constitutive or inducible. For example, in mammalian systems, the mouse metallothionein promoter can be induced by the addition of heavy metal ions.

The particulars for the construction of expression systems suitable for desired hosts are known to those in the art. Briefly, for recombinant production of the protein, the DNA encoding the polypeptide is suitably ligated into the expression vector of choice. The DNA is operably linked to all regulatory elements that are necessary for expression of the DNA in the selected host. One having ordinary skill in the art can, using well known techniques, prepare expression vectors for recombinant production of the polypeptide.

The expression vector including the DNA that encodes the CD40 splice variant is used to transform the compatible host which is then cultured and maintained under conditions wherein expression of the foreign DNA takes place. The protein of the present invention thus produced is recovered from the culture, either by lysing the cells or from the culture medium as appropriate and known to those in the art. One having ordinary skill in the art can, using well known techniques, isolate the CD40 splice variant that is produced using such expression systems. The methods of purifying the CD40 splice variant from natural sources using antibodies that specifically bind to the CD40 splice variant as described above, may be equally applied to purifying the CD40 splice variant produced by recombinant DNA methodology.

Examples of genetic constructs include the CD40 splice variant coding sequence operably linked to a promoter that is functional in the cell line into which the constructs are transfected. Examples of constitutive promoters include promoters from cytomegalovirus or SV40. Examples of inducible promoters include mouse mammary leukemia virus or metallothionein promoters. Those having ordinary skill in the art can readily produce genetic constructs useful for transfecting with cells with DNA that encodes the CD40 splice variant from readily available starting materials. Such gene constructs are useful for the production of the CD40 splice variant.

In bacterial systems, a number of expression vectors may be selected depending upon the use intended for the CD40 splice variant product. For example, when large quantities of CD40 splice variant product are needed for the induction of antibodies, vectors which direct high level expression of fusion proteins that are readily purified may be desirable. Such vectors include, but are not limited to, multifunctional E. coli cloning and expression vectors such as Bluescript(R) (Stratagene), in which the CD40 splice variants polypeptides coding sequence may be ligated into the vector in-frame with sequences for the amino-terminal Met and the subsequent 7 residues of beta-galactosidase so that a hybrid protein is produced; pIN vectors (Van Heeke & Schuster J. Biol. Chem. 264:5503-5509, (1989)); pET vectors (Novagen, Madison Wis.); and the like. In some embodiments, for example, one having ordinary skill in the art can, using well known techniques, insert such DNA molecules into a commercially available expression vector for use in well known expression systems. For example, the commercially available plasmid pSE420 (Invitrogen, San Diego, Calif.) may be used for production of collagen in E. coli.

In the yeast Saccharomyces cerevisiae a number of vectors containing constitutive or inducible promoters such as alpha factor, alcohol oxidase and PGH may be used. For reviews, see Ausubel et al. supra and Grant et al., Methods in Enzymology 153:516-544, (1987)). The commercially available plasmid pYES2 (Invitrogen, San Diego, Calif.) may, for example, be used for production in S. cerevisiae strains of yeast.

In cases where plant expression vectors are used, the expression of a sequence encoding variant products may be driven by any of a number of promoters. For example, viral promoters such as the 35S and 19S promoters of CaMV (Brisson et al., Nature 310:511-514 (1984)) may be used alone or in combination with the omega leader sequence from TMV (Takamatsu et al., EMBO J., 6:307-311, (1987)). Alternatively, plant promoters such as the small subunit of RUBISCO (Coruzzi et al., EMBO J. 3:1671-1680, (1984); Broglie et al., Science 224:838-843, (1984)); or heat shock promoters (Winter J and Sinibaldi R. M., Results Probl. Cell Differ., 17:85-105, (1991)) may be used. These constructs can be introduced into plant cells by direct DNA transformation or pathogen-mediated transfection. For reviews of such techniques, see Hobbs S. or Murry L. E. (1992) in McGraw Hill Yearbook of Science and Technology, McGraw Hill, New York, N.Y., pp 191-196; or Weissbach and Weissbach (1988) Methods for Plant Molecular Biology, Academic Press, New York, N.Y., pp 421-463.

CD40 splice variants products may also be expressed in an insect system. In one such system, Autographa californica nuclear polyhedrosis virus (AcNPV) is used as a vector to express foreign genes in Spodoptera frugiperda cells or in Trichoplusia larvae. The CD40 splice variants products coding sequence may be cloned into a nonessential region of the virus, such as the polyhedrin gene, and placed under control of the polyhedrin promoter. Successful insertion of CD40 variants coding sequences will render the polyhedrin gene inactive and produce recombinant virus lacking coat protein coat. The recombinant viruses are then used to infect S. frugiperda cells or Trichoplusia larvae in which variant protein is expressed (Smith et al., J. Virol. 46:584, (1983); Engelhard et al., Proc. Nat. Acad. Sci. 91:3224-7, (1994)). The commercially available MAXBACJ complete baculovirus expression system (Invitrogen, San Diego, Calif.) may, for example, be used for production in insect cells.

In mammalian host cells, a number of viral-based expression systems may be utilized. In cases where an adenovirus is used as an expression vector, CD40 splice variants products coding sequences may be ligated into an adenovirus transcription/translation complex consisting of the late promoter and tripartite leader sequence. Insertion in a nonessential E1 or E3 region of the viral genome will result in a viable virus capable of expressing variant protein in infected host cells (Logan and Shenk, Proc. Natl. Acad. Sci. 81:3655-59, (1984). In addition, transcription enhancers, such as the Rous sarcoma virus (RSV) enhancer, may be used to increase expression in mammalian host cells. The commercially available plasmid pcDNA I (Invitrogen, San Diego, Calif.) may, for example, be used for production in mammalian cells such as Chinese Hamster Ovary cells.

Specific initiation signals may also be required for efficient translation of variant products coding sequences. These signals include the ATG initiation codon and adjacent sequences. In cases where CD40 splice variant products coding sequence, its initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, D. et al., (1994) Results Probl. Cell Differ., 20:125-62, (1994); Bittner et al., Methods in Enzymol. 153:516-544, (1987)).

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis et al., (1986) Basic Methods in Molecular Biology). Cell-free translation systems can also be employed to produce polypeptides using RNAs derived from the DNA constructs of the present invention.

A host cell strain may be chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a Apre-pro” form of the protein may also be important for correct insertion, folding and/or function. Different host cells such as CHO, HeLa, MDCK, 293, WI38, etc. have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stablly express variant products may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clumps of stablely transformed cells can be proliferated using tissue culture techniques appropriate to the cell type.

Any number of selection systems may be used to recover transformed cell lines. These include, but are not limited to, the herpes simplex virus thymidine kinase (Wigler et al., Cell 11:223-32, (1977)) and adenine phosphoribosyltransferase (Lowy et al., Cell 22:817-23, (1980)) genes which can be employed in tk− or aprt− cells, respectively. Also, antimetabolite, antibiotic or herbicide resistance can be used as the basis for selection; for example, dhfr which confers resistance to methotrexate (Wigler, Proc. Natl. Acad. Sci. 77:3567-70, (1980)); npt, which confers resistance to the aminoglycosides neomycin and G-418 (Colbere-Garapin, J. Mol. Biol., 150: 1-14, (1981)) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci 85:8047-51, (1988)). The use of visible markers has gained popularity with such markers as anthocyanins, beta-glucuronidase and its substrate, GUS, and luciferase and its substrates, luciferin and ATP, being widely used not only to identify transformants, but also to quantify the amount of transient or stable protein expression attributable to a specific vector system (Rhodes et al., Methods Mol. Biol., 55:121-131, (1995)).

Host cells transformed with nucleotide sequences encoding CD40 splice variants products may be cultured under conditions suitable for the expression and recovery of the encoded protein from cell culture. The product produced by a recombinant cell may be secreted or contained intracellularly depending on the sequence and/or the vector used. As will be understood by those of skill in the art, expression vectors containing nucleic acid sequences encoding CD40 splice variants products can be designed with signal sequences which direct secretion of CD40 splice variants products through a prokaryotic or eukaryotic cell membrane.

The present invention relates to a transgenic non-human mammal that comprises the recombinant expression vector that comprises a nucleic acid sequence that encodes the CD40 splice variant that comprises the amino acid sequence of SEQ ID NO:2, SEQ ID NO:4, SEQ ID NO:6, SEQ ID NO:7, SEQ ID NO:8, SEQ ID NO:9, SEQ ID NO:10 and fragments and homologues. In some embodiments the transgene comprises SEQ ID NO:1, SEQ ID NO:3 or SEQ ID NO:5. Transgenic non-human mammals useful to produce recombinant proteins are well known as are the expression vectors necessary and the techniques for generating transgenic animals. Generally, the transgenic animal comprises a recombinant expression vector in which the nucleotide sequence that encodes a CD40 splice variant of the invention is operably linked to a mammary cell specific promoter whereby the coding sequence is only expressed in mammary cells and the recombinant protein so expressed is recovered from the animal's milk.

In some embodiments of the invention, transgenic non-human animals are generated. The transgenic animals according to the invention contain the coding sequence that encodes a CD40 splice variant, such as SEQ ID NO:1, SEQ ID NO:3 or SEQ ID NO:5, under the regulatory control of a mammary specific promoter. One having ordinary skill in the art using standard techniques, such as those taught in U.S. Pat. No. 4,873,191 issued Oct. 10, 1989 to Wagner and U.S. Pat. No. 4,736,866 issued Apr. 12, 1988 to Leder, both of which are incorporated herein by reference, can produce transgenic animals which produce the CD40 splice variant. Preferred animals are rodents, particularly, rats and mice, or goats.

In some embodiments, the CD40 splice variant protein may be expressed as a recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp, Seattle, Wash.).

The inclusion of a protease-cleavable polypeptide linker sequence between the purification domain and CD40 splice variant is useful to facilitate purification. One such expression vector provides for expression of a fusion protein compromising a variant polypeptide fused to a polyhistidine region separated by an enterokinase cleavage site. The histidine residues facilitate purification on IMIAC (immobilized metal ion affinity chromatography, as described in Porath et al., Protein Expression and Purification, 3:263-281, (1992)) while the enterokinase cleavage site provides a means for isolating variant polypeptide from the fusion protein. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Following transformation of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can by disrupted by any convenient method, including freeze-thaw cycling, sonication, mechanical disruption, or use of cell lysing agents, or other methods, which are well know to those skilled in the art.

The CD40 splice variant can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, phosphocellulose chromatography, hydrophobic interation chromatography, affinity chromatography, hydroxylapatite chromatography, and lectin chromatography. Protein refolding steps can be used, as necessary, in completing configuration of the mature protein. Finally, high performance liquid chromatography (HPLC) can be employed for final purification steps. In some embodiments, antibodies may be used to isolate CD40 splice variant proteins.

In addition to producing these proteins by recombinant techniques, automated peptide synthesizers may also be employed to produce CD40 splice variants of the invention. Such techniques are well known to those having ordinary skill in the art and are useful if derivatives which have substitutions not provided for in DNA-encoded protein production. CD40 splice variants, fragments and portions of variant products may be produced by direct peptide synthesis using solid-phase techniques (cf. Stewart et al., (1969) Solid-Phase Peptide Synthesis, WH Freeman Co, San Francisco; Merrifield, J Am Chem. Soc., 85:2149-2154, (1963)). In vitro peptide synthesis may be performed using manual techniques or automation. Automated synthesis may be achieved, e.g., using Applied Biosystems 431A Peptide Synthesizer (Perkin Elmer, Foster City, Calif.) in accordance with the instructions provided by the manufacturer. Fragments of CD40 splice variants may be chemically synthesized separately and combined using chemical methods to produce the full length molecule.

In Vivo Applications

Soluble CD40 splice variants and gene therapeutics which encode such proteins may be used in the treatment of a number of diseases, disorders and conditions which can be cured or ameliorated by lowering the level of any of the CD40 ligands. Antisense molecules which inhibit expression of CD40 variants and antibodies specific for CD40 variants may also be useful in treatment of disease, disorder, pathological or normal condition involving CD40 such as inflammatory diseases, autoimmune diseases involving the immune system. Some embodiments of the present invention provide pharmaceutical compositions comprising, as an active ingredient, the nucleic acid molecules, expression vectors, recombinant host cells, protein, antibodies and hybridomas described herein. The present invention also provides pharmaceutical compositions comprising, as an active ingredient, the nucleic acid molecules which comprise or consist of said complementary sequences, or of a vector comprising said complementary sequences.

CD40 splice variant proteins may modulate CD40-CD154 interactions. The modulation of CD40-CD154 interactions may modulate activation of B- and T-lymphocytes including those activations associated with chronic inflammatory diseases such as graft-versus-host disease, transplant rejection, neurodegenerative disorders, atherosclerosis, pulmonary fibrosis, autoimmune diseases such as lupus nephritis, systemic lupus erythematosus, rheumatoid arthritis, multiple sclerosis, as well as hematological malignancies and other cancers. Similarly, in some embodiments nucleic acid molecules are provided which encode the CD40 splice variant. These nucleic acid molecules are delivered to individuals as therapeutics where they are taken up by cells and expressed, thus producing the CD40 splice variant protein which thereby modulates of CD40-CD 154 interactions and effects activation of B- and T-cells to have a therapeutic effect. In some embodiments, pharmaceutical compositions comprising antibodies or antisense molecules are administered to the individual to either inhibit action of the CD40 splice variant protein present in the individual or inhibit its production.

The soluble CD40 splice variants and gene therapeutics which encode such proteins may block or reduce various chronic inflammatory conditions by disrupting the CD40-CD154 system. In some embodiments, the invention relates to methods of treating individuals suffering from autoimmune diseases and disorders. T-cell mediated autoimmune diseases include Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. B-cell mediated autoimmune diseases include Lupus (SLE), Grave's disease, myasthenia gravis, autoimmune hemolytic anemia, autoimmune thrombocytopenia, asthma, cryoglobulinemia, primary biliary sclerosis and pernicious anemia. In some preferred embodiments, the soluble CD40 splice variants and gene therapeutics which encode such proteins is used to treat prevent or reduce the severity of injury and inflammation or symptoms or progression of the same. By modulating the CD40-CD154 interaction in T-cell priming and tolerance induction, the soluble CD40 splice variants and gene therapeutics which encode such proteins may be used in association with allografts, such as skin, cardiac, renal, islet and bone marrow, in order to reduce transplantation rejection and prolong allograft survival or to treat, prevent or reduce the severity of graft versus host diseases. Disruption of the CD40-CD154 pathway using soluble CD40 splice variants and gene therapeutics that encode such proteins can be used in the treatment of atherosclerosis, prevent atherosclerotic progression and may reverse established lesions. Similarly, disruption of the CD40-CD154 pathway treatment using soluble CD40 splice variants and gene therapeutics which encode such proteins may mediate many of the key events involved in fibrogenesis and be useful in the treatment of acute injury by for example reducing inflammation and avoiding progression to end-stage fibrosis. Examples of such injuries include soluble CD40 splice variants and gene therapeutics which encode such proteins hyperoxic injuries such as hyperoxic lung injury and radiation-induced injuries such as radiation induced lung injury. Similarly, disruption of the CD40-CD154 pathway treatment using soluble CD40 splice variants and gene therapeutics which encode such proteins may be used in the treatment of inflammatory bowel diseases (IBD), ulcerative colitis and Crohn's disease. The treatment of B-cell lymphoid malignancies as well as epithelial neoplasia, nasopharyngeal carcinoma, osteosarcoma, neuroblastoma and bladder carcinoma. Those having ordinary skill in the art can readily identify individuals who are suspected of suffering from such diseases, conditions and disorders using standard diagnostic techniques.

The ability of the CD40 splice variant proteins to inhibit atherosclerosis in vivo can be examined studied using animal models known in the art, e.g., as described Purcell-Huynh et al., J. Clin. Invest. 95:2246-257 (1995); Fruebis et al., J. Clin. Invest. 94:392-98 (1994); van Ree et al., Biochem J. 305:905-11 (1995); Masucci et al., Science 275:391-394 (1997); Chowdhury et al., Science 254:1802-805 (1991); Tangirala et al., J. Lipid Res. 36:2320-328 (1995); Ishibashi et al., J. Clin. Invest. 93:1885-893 (1994). For example, in the LDLR^{-/-} mouse model of Litchman et al., FASEB J. 154:896 (1997), atherosclerotic lesion formation is initiated in a defined manner through dietary manipulation.

In some embodiments, male mice are used in the trial. Preferably, 10, 25, 50, 100, 200, of 500 or more mice are used in the trial. In one illustrative trial, 100 mice are tested. Atherosclerosis is induced in half of the mice one hundred mice (atherosclerosis mice) by dietary manipulation and the remaining one hundred mice are fed a normal diet (non-atherosclerosis mice). Purified splice variant proteins of the present invention are administered systemically by intravenous injection during the period of atherogenesis to fifty of the atherosclerosis mice (treated atherosclerotic mice) and fifty of the non-atherosclerosis mice (treated non-atherosclerotic mice). A control solution is injected into the remaining fifty atherosclerotic mice (control atherosclerotic mice) and the remaining fifty non-atherosclerotic mice (control non-atherosclerotic mice). Ten animals from each group are sacrificed at 15, 30, 60, 90 and 120 days. The hearts and aortas of the mice are collected and analyzed with immunohistochemistry to quantify lesional development.

In another trial, the CD40 splice variant proteins of the present invention are expressed in vivo by an expression construct in the mice described above. The sequences encoding the splice variant proteins of the present invention are cloned into an appropriate gene therapy vector downstream of an operable promoter. Two hundred mice are used in the trial. Atherosclerosis is induced in one hundred mice (atherosclerosis mice) by dietary manipulation and the remaining one hundred mice are fed a normal diet (non-atherosclerosis mice). The vector is administered to fifty of the atherosclerosis mice (treated atherosclerotic mice) and fifty of the non-atherosclerosis mice (treated non-atherosclerotic mice). A control solution is injected into the remaining fifty atherosclerotic mice (control atherosclerotic mice) and the remaining fifty non-atherosclerotic mice (control non-atherosclerotic mice). Ten animals from each group are sacrificed at 15, 30, 60, 90 and 120 days. The hearts and aortas of the mice are collected and analyzed with immunohistochemistry to quantify lesional development.

Pharmaceutical compositions according to some embodiments of the invention comprise a pharmaceutically acceptable carrier in combination with a CD40 splice variant protein, a nucleic acid that encodes CD40 splice variant protein, an antibody that specifically binds to the CD40 splice variant or an antisense nucleic acid molecule that inhibits production or expression of the transcript that encodes the CD40 splice variant. Pharmaceutical formulations are well known and pharmaceutical compositions comprising on of the aforementioned active ingredient of the invention may be routinely formulated by one having ordinary skill in the art. Suitable pharmaceutical carriers are described in Remington's Pharmaceutical Sciences, A. Osol, a standard reference text in this field, which is incorporated herein by reference. The present invention relates to an injectable pharmaceutical composition. Such embodiments are necessarily sterile and pyrogen free. Some embodiments of the invention relate to injectable pharmaceutical compositions that comprise a pharmaceutically acceptable carrier and amino acid sequence that is SEQ ID NO:2, SEQ ID NO:4, SEQ ID NO:6, SEQ ID NO:7, SEQ ID NO:8, SEQ ID NO:9, SEQ ID NO:10, fragments, homologues or fragments of homologues thereof. Some embodiments of the invention relate to injectable pharmaceutical compositions that comprise a pharmaceutically acceptable carrier and a nucleic acid molecule that encodes an amino acid sequence that is SEQ ID NO:2, SEQ ID NO:4, SEQ ID NO:6, SEQ ID NO:7, SEQ ID NO:8, SEQ ID NO:9, SEQ ID NO:10 fragments, homologues or fragments of homologues thereof. Some embodiments of the invention relate to injectable pharmaceutical compositions that comprise a pharmaceutically acceptable carrier and an antibody that specifically binds to a protein with an amino acid sequence that is SEQ ID NO:2, SEQ ID NO:4, SEQ ID NO:6, SEQ ID NO:7, SEQ ID NO:8, SEQ ID NO:9, SEQ ID NO:10, fragments, homologues or fragments of homologues thereof. Some embodiments of the invention relate to injectable pharmaceutical compositions that comprise a pharmaceutically acceptable carrier and antisense nucleic acid molecules that specifically inhibit expression of nucleic acid molecules that encode an amino acid sequence that is SEQ ID NO:2, SEQ ID NO:4, SEQ ID NO:6, SEQ ID NO:7, SEQ ID NO:8, SEQ ID NO:9 or SEQ ID NO:10.

In some embodiments, for example, the CD40 splice variant protein, nucleic acid molecule, antibody or antisense compound can be formulated as a solution, suspension, emulsion, ointment, gel, suppository or lyophilized powder in association with a pharmaceutically acceptable vehicle. Examples of such vehicles are water, saline, buffered saline, Ringer's solution, dextrose solution, 5% human serum albumin, glycerol, ethanol, and combinations thereof. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The product of the invention may also be used to modulate endothelial differentiation and proliferation as well as to modulate apoptosis either ex vivo or in vitro, for example, in cell cultures. The formulation is sterilized by commonly used techniques.

An injectable composition may comprise the CD40 splice variant protein, nucleic acid molecule, antibody or antisense compound in a diluting agent such as, for example, sterile water, electrolytes/dextrose, fatty oils of vegetable origin, fatty esters, or polyols, such as propylene glycol and polyethylene glycol. The injectable must be sterile and free of pyrogens.

Pharmaceutical compositions according to the invention include delivery components in combination with nucleic acid molecules that encode a CD40 splice variant protein which further comprise a pharmaceutically acceptable carriers or vehicles, e.g., saline. Any medium may be used which allows for successful delivery of the nucleic acid. One skilled in the art would readily comprehend the multitude of pharmaceutically acceptable media that may be used in the present invention.

The pharmaceutical compositions of the present invention may be administered by any means that enables the active agent to reach the agent's site of action in the body of a mammal. The pharmaceutical compositions of the present invention may be administered by any of a number of routes and methods designed to provide a consistent and predictable concentration of compound at the target organ or tissue. The compositions may be administered alone in combination with other agents, such as stabilizing compounds, and/or in combination with other pharmaceutical agents such as drugs or hormones. The compositions may be administered by a number of routes including, but not limited to oral, intravenous, intramuscular, transdermal, subcutaneous, topical, by absorption through epithelial or mucocutaneous linings, for example, nasal, oral, vaginal, rectal, gastrointestinal and sublingual. Pharmaceutical compositions may be administered parenterally, i.e., intravenous, subcutaneous, intramuscular, intraperitoneal. The product may be injected to other localized regions of the body. The product may also be administered via nasal insufflation. Enteral administration is also possible. For such administration, the product should be formulated into an appropriate capsule or elixir for oral administration, or into a suppository for rectal administration. Intravenous administration is the preferred route. In some preferred embodiments, the protein, nucleic acid molecule, antibody or antisense compound is administered typically as a sterile solution by IV injection, although other parenteral routes may be suitable.

Dosage varies depending upon known factors such as the pharmacodynamic characteristics of the particular agent, and its mode and route of administration; age, health, and weight of the recipient; nature and extent of symptoms, kind of concurrent treatment, frequency of treatment, the effect desired, the potency and therapeutic index of the active agent.

In some therapeutic applications, CD40 splice variant proteins are administered in an amount between about 5 [symbol] g to 5000 mg of protein. In some preferred embodiments, 50 [symbol] g to 500 mg of protein may be administered. In other preferred embodiments, 500 [symbol] g to 50 mg of protein may be administered. In a preferred embodiment, 5 mg of protein is administered. Treatment may be continued, e.g., with dosing every 1-7 days, until a therapeutic improvement is seen.

In some therapeutic applications, the antibody employed is preferably a humanized monoclonal antibody, or a human Mab produced by known globulin-gene library methods. Typically, the antibody is administered in an amount between about 1-15 mg/kg body weight of the subject. Treatment is continued, e.g., with dosing every 1-7 days, until a therapeutic improvement is seen.

In Vitro Applications

The detection of CD40 variant expression are useful in screening, diagnostic and monitoring protocols, i.e., their presence or level may be indicative of a disease, disorder, pathological or normal condition involving CD40 such as inflammatory diseases, autoimmune diseases involving the immune system, and other pathological conditions. Examples of disease, disorder, pathological or normal condition involving CD40 such as inflammatory diseases, autoimmune diseases involving the immune system, and other pathological conditions include chronic inflammatory diseases such as graft-versus-host disease, transplant rejection, neurodegenerative disorders, atherosclerosis, pulmonary fibrosis, autoimmune diseases such as lupus nephritis, systemic lupus erythematosus, rheumatoid arthritis, multiple sclerosis, inflammatory bowel diseases (IBD), ulcerative colitis and Crohn's disease as well as hematological malignancies and other cancers. In some embodiments, autoimmune diseases and disorders refer to T-cell mediated autoimmune diseases such as Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. In some embodiments, autoimmune diseases and disorders refer to B-cell mediated autoimmune diseases include Lupus (SLE), Grave's disease, myasthenia gravis, autoimmune hemolytic anemia, autoimmune thrombocytopenia, asthma, cryoglobulinemia, primary biliary sclerosis and pernicious anemia. In some embodiments, the disease, disorder, or pathological condition involves severity of injury and inflammation or symptoms or progression of the same. In some embodiments, the disease, disorder, or pathological condition involves transplantation rejection and prolonged survival of allografts, such as skin, cardiac, renal, islet and bone marrow or graft versus host diseases. In some embodiments, the disease, disorder, or pathological condition involves atherosclerosis, atherosclerotic progression, fibrogenesis, acute injury, end-stage fibrosis, and hyperoxic injuries such as hyperoxic lung injury and radiation-induced injuries such as radiation induced lung injury. Detection of expression of the CD40 splice variants may be useful in the screening, diagnosing and treatment monitoring of individuals who have various forms of cancer, such as for example epithelial neoplasia, nasopharyngeal carcinoma, osteosarcoma, neuroblastoma and bladder carcinoma. In addition, detection of expression of the CD40 splice variants of the invention may be useful in the screening, diagnosing and treatment monitoring of individuals who have AIDS-related lymphoma, or impaired renal function, including chronic renal failure, haemodialysis and chronic ambulatory peritoneal dialysis (CAPD) patients.

Alternatively, the ratio between the variants' level and the level of the original CD40 from which they have been varied, or the ratio of any variants with respect to each other may be indicative to such a disease, disorder, pathological or normal condition. It is possible, e.g., to establish differential expression of CD40 variants in various tissues as compared to the original CD40. The variants may be expressed mainly in one tissue, while the original CD40 sequence from which they have been varied, may be expressed mainly in another tissue. Understanding of the distribution of the variants as compared to the original sequence or as compared to one another in various tissues may be helpful in basic research for understanding the physiological function of the gene as well as for helping target pharmaceuticals or developing pharmaceuticals. The presence or the level of expression of the CD40 variants may be determined within a specific cell population, comparing said presence or level between various cell types in a tissue, between different tissues and between individuals. Some embodiments of the invention relate to methods of screening, diagnostic and monitoring individuals. Some embodiments of the invention relate to reagents and kits useful in such methods.

In some embodiments of the invention, diagnostic methods and kits of the present invention are specifically designed to detect evidence of expression of CD40 splice variants, either by detecting the protein itself or the nucleic acid transcript that encodes it.

According to some embodiments of the invention, antibodies are provided that bind to epitopes, which include amino acid residues of the unique tail sequence of a CD40 splice variant. Alternatively, mRNA encoding the CD40 splice variant, or cDNA generated therefrom, may be detected as evidence of expression of the CD40 splice variant. The marker may be useful in methods of screening, diagnosing and monitoring such diseases conditions and disorders. Kits are provides to perform the methods of the invention.

Individuals who are at risk for developing particular diseases, conditions or disorders may be screened using the in vitro diagnostic methods of the present invention. The invention is particularly useful for monitoring individuals whose family medical history includes relatives who have suffered from such diseases, conditions or disorders. Further, the methods may be used to diagnose patients who exhibit other symptoms of the such diseases conditions or disorders or to confirm diagnosis in combination with other tests and observations. Likewise, the invention is useful to monitor individuals who have been diagnosed as having such diseases, conditions or disorders and, who are being treated to determine if they are responding to therapy or who have been treated to detect recurrence.

Samples may be obtained from any tissue or body fluid. Body fluid samples are preferred. Examples of body fluid samples include blood, urine, lymph fluid, cerebral spinal fluid, amniotic fluid, vaginal fluid and semen. In some preferred embodiments, blood is used as a sample of body fluid. Blood may be processed to serum. One skilled in the art would readily appreciate the variety of test samples that may be examined. Test samples may be obtained by such methods as withdrawing fluid with a syringe or by a swab or by collecting fluid from any number of other well established techniques. One skilled in the art would readily recognize other methods of obtaining test samples.

In an assay using a blood sample, the blood plasma may be separated from the blood cells. In some embodiments, the blood plasma may be screened for CD40 splice variant protein that is released into the blood. In some embodiments, samples are screened to detect the presence of mRNA encoding the protein.

Protein Based Assays

In some embodiments, the present invention relates to a method for detecting the CD40 splice variant in a biological sample. The method includes (a) contacting with the biological sample the antibody of the invention, thereby forming an antibody-antigen complex; and (b) detecting the antibody-antigen complex. The presence of the antibody-antigen complex correlates with the presence of the CD40 splice variants products in said biological sample. As indicated above, the method can be quantitated to determine the level or the amount of the CD40 splice variants in the sample, alone or in comparison to the level of the original CD40 amino acid sequence from which it was varied. Additionally, qualitative and quantitative results may be used for diagnostic, prognostic and therapeutic planning purposes.

Some embodiments of the present invention relate to immunoassay methods of identifying individuals suffering from particular diseases, conditions or disorders by detecting the presence of one or more CD40 splice variant proteins in a sample of tissue or bodily fluid using antibodies that specifically bind to an epitope, which include amino acid residues of the unique tail of the CD40 splice variant. The antibodies do not cross react with CD40 (SEQ ID NO: 24). According to another aspect of the invention, the present invention provides methods for detecting, comparing and monitoring levels of expression of CD40 splice variants in a bodily fluid sample, or in a specific tissue sample, e.g., by the use of antibodies capable of specifically reacting with the CD40 splice variant of the invention. Detection of the level of the expression of the CD40 variants of the invention in particular may be indicative of a plurality of physiological or pathological conditions.

The antibodies are preferably monoclonal antibodies. The antibodies are preferably raised against CD40 splice variant protein made in human cells. Immunoassays are well known and their design may be routinely undertaken by those having ordinary skill in the art. Antibodies of the invention and the methods in which they may be produced are described above.

The means to detect the presence of a protein in a test sample are routine and one having ordinary skill in the art can detect the presence or absence of a protein or an antibody using well-known methods. One well-known method of detecting the presence of a protein is an immunoassay. One having ordinary skill in the art can readily appreciate the multitude of ways to practice an immunoassay to detect the presence of a CD40 splice variant protein in a sample.

According to some embodiments, immunoassays include allowing proteins in the sample to bind a solid phase support such as a plastic surface. Detectable antibodies that selectively bind to CD40 splice variant proteins are then added. Detection of the detectable antibody indicates the presence of CD40 splice variant protein. The detectable antibody may be a labeled or an unlabeled antibody. Unlabeled antibody may be detected using a second, labeled antibody that specifically binds to the first antibody or a second, unlabeled antibody that can be detected using labeled protein A, a protein that complexes with antibodies. Various immunoassay procedures are described in Immunoassays for the 80's, A. Voller et al., Eds., University Park, 1981, which is incorporated herein by reference.

Simple immunoassays may be performed in which a solid phase support is contacted with the test sample. Any proteins present in the test sample bind the solid phase support and can be detected by a specific, detectable antibody preparation. Such a technique is the essence of the dot blot, Western blot and other such similar assays.

Other immunoassays may be more complicated but actually provide excellent results. Typical and preferred immunometric assays include “forward” assays for the detection of a protein, in which a first anti-protein antibody bound to a solid phase support is contacted with the test sample. After a suitable incubation period, the solid phase support is washed to remove unbound protein. A second, distinct anti-protein antibody is then added which is specific for a portion of the specific protein not recognized by the first antibody. The second antibody is preferably detectable. After a second incubation period to permit the detectable antibody to complex with the specific protein bound to the solid phase support through the first antibody, the solid phase support is washed a second time to remove the unbound detectable antibody. Alternatively, the second antibody may not be detectable. In this case, a third detectable antibody, which binds the second antibody is added to the system. This type of “forward sandwich” assay may be a simple yes/no assay to determine whether binding has occurred or may be made quantitative by comparing the amount of detectable antibody with that obtained in a control. Such “two-site” or “sandwich” assays are described by Wide, Radioimmune Assay Method, Kirkham, Ed., E. & S. Livingstone, Edinburgh, 1970, pp. 199-206, which is incorporated herein by reference.

Other types of immunometric assays are the so-called “simultaneous” and “reverse” assays. A simultaneous assay involves a single incubation step wherein the first antibody bound to the solid phase support, the second, detectable antibody and the test sample are added at the same time. After the incubation is completed, the solid phase support is washed to remove unbound proteins. The presence of detectable antibody associated with the solid support is then determined as it would be in a conventional “forward sandwich” assay. The simultaneous assay may also be adapted in a similar manner for the detection of antibodies in a test sample.

The “reverse” assay comprises the stepwise addition of a solution of detectable antibody to the test sample followed by an incubation period and the addition of antibody bound to a solid phase support after an additional incubation period. The solid phase support is washed in conventional fashion to remove unbound protein/antibody complexes and unreacted detectable antibody. The determination of detectable antibody associated with the solid phase support is then determined as in the “simultaneous” and “forward” assays. The reverse assay may also be adapted in a similar manner for the detection of antibodies in a test sample.

The first component of the immunometric assay may be added to nitrocellulose or other solid phase support which is capable of immobilizing proteins. The first component for determining the presence of a CD40 splice variant protein in a test sample is antibody specific for the CD40 splice variant protein. By “solid phase support” or “support” is intended any material capable of binding proteins. Well-known solid phase supports include glass, polystyrene, polypropylene, polyethylene, dextran, nylon, amylases, natural and modified celluloses, polyacrylamides, agaroses, and magnetite. The nature of the support can be either soluble to some extent or insoluble for the purposes of the present invention. The support configuration may be spherical, as in a bead, or cylindrical, as in the inside surface of a test tube or the external surface of a rod. Alternatively, the surface may be flat such as a sheet, test strip, etc. Those skilled in the art will know many other suitable “solid phase supports” for binding proteins or will be able to ascertain the same by use of routine experimentation. A preferred solid phase support is a 96-well microtiter plate.

According to some embodiments of the invention, antibodies can be detectably labeled is by linking the antibodies to an enzyme and subsequently using the antibodies in an enzyme immunoassay (EIA) or enzyme-linked immunosorbent assay (ELISA), such as a capture ELISA. The enzyme, when subsequently exposed to its substrate, reacts with the substrate and generates a chemical moiety which can be detected by, e.g., spectrophotometric, fluorometric or visual means. Enzymes which can be used to detectably label antibodies include, e.g., malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. One skilled in the art would readily recognize other enzymes that may also be used.

Another method in which antibodies can be detectably labeled is through radioactive isotopes and subsequent use in a radioimmunoassay (RIA) (see, e.g., Work, Laboratory Techniques and Biochemistry in Molecular Biology, North Holland Publishing Company, N.Y., 1978, which is incorporated herein by reference). The radioactive isotope can be detected by such means as the use of a gamma counter or a scintillation counter or by autoradiography. Isotopes which are particularly useful for the purpose of the present invention are ³H, ¹²⁵I, ¹³¹I, ³⁵S, and ¹⁴C. Preferably ¹²⁵I is the isotope. One skilled in the art would readily recognize other radioisotopes which may also be used.

It is also possible to label the antibody with a fluorescent compound. When the fluorescent-labeled antibody is exposed to light of the proper wave length, its presence can be detected due to its fluorescence. Among the most commonly used fluorescent labeling compounds are fluorescein isothiocyanate, rhodamine, phycoerythrin, phycocyanin, allophycocyanin, o-phthaldehyde and fluorescamine. One skilled in the art would readily recognize other fluorescent compounds that may also be used.

Antibodies can also be detectably labeled using fluorescence-emitting metals such as ¹⁵²Eu, or others of the lanthanide series. These metals can be attached to the protein-specific antibody using such metal chelating groups as diethylenetriaminepentaacetic acid (DTPA) or ethylenediamine-tetraacetic acid (EDTA). One skilled in the art would readily recognize other fluorescence-emitting metals as well as other metal chelating groups that may also be used.

Antibody can also be detectably labeled by coupling to a chemiluminescent compound. The presence of the chemiluminescent-labeled antibody is determined by detecting the presence of luminescence that arises during the course of a chemical reaction. Examples of particularly useful chemoluminescent labeling compounds are luminol, isoluminol, theromatic acridinium ester, imidazole, acridinium salt and oxalate ester. One skilled in the art would readily recognize other chemiluminescent compounds that may also be used.

Likewise, a bioluminescent compound may be used to label antibodies. Bioluminescence is a type of chemiluminescence found in biological systems, in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin. One skilled in the art would readily recognize other bioluminescent compounds that may also be used.

Detection of the protein-specific antibody, fragment or derivative may be accomplished by a scintillation counter if, e.g., the detectable label is a radioactive gamma emitter. Alternatively, detection may be accomplished by a fluorometer if, e.g., the label is a fluorescent material. In the case of an enzyme label, the detection can be accomplished by colorometric methods that employ a substrate for the enzyme. Detection may also be accomplished by visual comparison of the extent of enzymatic reaction of a substrate in comparison with similarly prepared standards. One skilled in the art would readily recognize other appropriate methods of detection that may also be used.

The binding activity of a given lot of antibodies may be determined according to well known methods. Those skilled in the art will be able to determine operative and optimal assay conditions for each determination by employing routine experimentation.

Positive and negative controls may be performed, in which known amounts of CD40 splice variant protein and no CD40 splice variant proteins, respectively, are added to assays being performed in parallel with the test assay. One skilled in the art would have the necessary knowledge to perform the appropriate controls.

CD40 splice variant protein may be produced as a reagent for positive controls routinely. One skilled in the art would appreciate the different manners in which the CD40 splice variant protein may be produced and isolated.

To examine a test sample for the presence of the CD40 splice variant protein, a standard immunometric assay such as the one described below may be performed. A first antibody specific for the CD40 splice variant protein, which recognizes a specific portion of the CD40 splice variant protein unique tail, is added to a 96-well microtiter plate in a volume of buffer. The plate is incubated for a period of time sufficient for binding to occur and subsequently washed with PBS to remove unbound antibody. The plate is then blocked with a PBS/BSA solution to prevent sample proteins from nonspecifically binding the microtiter plate. Test samples are subsequently added to the wells and the plate is incubated for a period of time sufficient for binding to occur. The wells are washed with PBS to remove unbound protein. Labeled antibodies which recognize portions of the CD40 splice variant protein not recognized by the first antibody, are added to the wells. The plate is incubated for a period of time sufficient for binding to occur and subsequently washed with PBS to remove unbound, labeled anti-CD40 splice variant antibody. The amount of labeled and bound anti-CD40 splice variant antibody is subsequently determined by standard techniques.

Kits that are useful for the detection of a CD40 splice variant protein in a test sample include a container comprising anti-CD40 splice variant antibodies and a container or containers including controls. Controls include one control sample that does not contain CD40 splice variant protein and/or another control sample that contains CD40 splice variant protein. The antibodies used in the kit are detectable, e.g., detectably labeled. If the detectable antibody is not labeled, it may be detected by a second antibody or protein A, which may also be provided in some kits in separate containers. Additional components in some kits include solid support, buffer, graphics or photographs depicting positive and/or negative results and instructions for carrying out the assay.

The present invention relates to methods of identifying individuals suffering from particular diseases, disorders or conditions by detecting presence of a CD40 splice variant protein in sample using Western blots. Western blots use detectable antibodies to bind to CD40 splice variant protein in sample of tissue or body fluid using antibodies which specifically bind to an epitope which include amino acid residues of the unique tail of the CD40 splice variant. The antibodies do not cross react with wild type CD40.

Western blot techniques, which are described in Sambrook, J. et al., (1989) Molecular Cloning: A Laboratory Manual, Cold Spring Harbor Laboratory Press, Cold Spring Harbor, N.Y., which is incorporated herein by reference, are similar to immunoassays with the essential difference being that prior to exposing the sample to the antibodies, the proteins in the samples are separated by gel electrophoresis and the separated proteins are then probed with antibodies. In some preferred embodiments, the matrix is an SDS-PAGE gel matrix and the separated proteins in the matrix are transferred to a carrier such as filter paper prior to probing with antibodies. Antibodies described above are useful in Western blot methods.

Kits which are useful for the detection of CD40 splice variant protein in a test sample by Western Blot comprise a container comprising anti-CD40 splice variant antibodies and a container or containers comprising controls. Controls include one control sample which does not contain the CD40 splice variant and/or another control sample which contains the CD40 splice variant protein. The antibodies used in the kit are detectable such as being detectably labeled. If the detectable anti-ST antibody is not labeled, it may be detected by second antibodies or protein A for example which may also be provided in some kits in separate containers. Additional components in some kits include buffer, graphics or photographs depicting positive and/or negative results and instructions for carrying out the assay.

Nucleic Acid Based Assays

According to another aspect of the invention, the present invention provides methods for detecting the level of the transcripts (mRNA) of said CD40 splice variants product in a body fluid sample, or in a specific tissue sample, e.g., by use of probes comprising or consisting of said coding sequences. Aspects of the present invention include various methods of determining whether a sample contains transcript that encodes a CD40 splice variant. Several different methods are available for doing so including those using Polymerase Chain Reaction (PCR) technology, using Northern blot technology, oligonucleotide hybridization technology, and in situ hybridization technology. Quantitative detection of the level of the expression of the CD40 variants of the invention in particular may be indicative of a plurality of physiological or pathological conditions.

The invention also relates to oligonucleotide probes and primers used in the methods of identifying mRNA that encodes a CD40 splice variant and to diagnostic kits which comprise such components. The mRNA sequence-based methods for determining whether a sample mRNA encoding a CD40 splice variant include but are not limited to polymerase chain reaction technology, Northern and Southern blot technology, in situ hybridization technology and oligonucleotide hybridization technology.

The methods described herein are meant to exemplify how the present invention may be practiced and are not meant to limit the scope of invention. It is contemplated that other sequence-based methodology for detecting the presence of specific mRNA that encodes a CD40 splice variant in a sample may be employed according to the invention.

A preferred method to detecting mRNA that encodes a CD40 splice variant in a sample uses polymerase chain reaction (PCR) technology. PCR technology is practiced routinely by those having ordinary skill in the art and its uses in diagnostics are well known and accepted. Methods for practicing PCR technology are disclosed in “PCR Protocols: A Guide to Methods and Applications”, Innis, M. A., et al. Eds. Academic Press, Inc. San Diego, Calif. (1990), which is incorporated herein by reference. Applications of PCR technology are disclosed in “Polymerase Chain Reaction” Erlich et al., Eds. Cold Spring Harbor Press, Cold Spring Harbor, N.Y. (1989), which is incorporated herein by reference. U.S. Pat. Nos. 4,683,202, 4,683,195, 4,965,188 and 5,075,216, each of which are incorporated herein by reference, describe methods of performing PCR. PCR may be routinely practiced using Perkin Elmer Cetus GENE AMP RNA PCR kit, Part No. N808-0017.

Some simple rules aid in the design of efficient primers. Typical primers are 18-28 nucleotides in length having 50% to 60% g+c composition. The entire primer is preferably complementary to the sequence to which it must hybridize. Preferably, primers generate PCR products from 100 base pairs to 2000 base pairs. However, it is possible to generate PCR products from 50 base pairs to more than 10 kb.

PCR technology allows for the rapid generation of multiple copies of nucleotide sequences by providing 5′ and 3′ primers that hybridize to sequences present in a nucleic acid molecule, and further providing free nucleotides and an enzyme which fills in the complementary bases to the nucleotide sequence between the primers with the free nucleotides to produce a complementary strand of DNA. The enzyme will fill in the complementary sequences adjacent to the primers. If both the 5′ primer and 3′ primer hybridize to nucleotide sequences on the complementary strands of the same fragment of nucleic acid, exponential amplification of a specific double-stranded product results. If only a single primer hybridizes to the nucleic acid molecule, linear amplification produces single-stranded products of variable length.

To perform this method, cDNA is reverse-transcribed from extracted RNA using well known methods and readily available starting materials.

The mRNA or cDNA is combined with the primers, free nucleotides and enzyme following standard PCR protocols. The mixture undergoes a series of temperature changes. If the mRNA or cDNA encoding a CD40 splice variant is present, i.e., if both primers hybridize to sequences on the same molecule, the molecule comprising the primers and the intervening complementary sequences will be exponentially amplified. The amplified DNA can be easily detected by a variety of well known means. If the chimeric gene is not present, no DNA molecule will be exponentially amplified. Rather, amplification of wild-type transcript will yield low levels of variable length product. The PCR technology therefore provides an extremely easy, straightforward and reliable method of detecting mRNA encoding a CD40 splice variant in a sample.

PCR primers can be designed routinely by those having ordinary skill in the art using well known cDNA sequence information. At least one primer corresponds to the sequence that encodes at least a portion of the unique tail, or a sequence complementary thereto, such that the primer when is used it will only amplify transcripts encoding the unique tail of a CD40 splice variant. Accordingly, the portion of sequence encoding the unique tail must be sufficient to allow selective amplifcation of the CD40 splice variant. Such a portion is preferably at least 8, more preferably at least 10, more preferably at least 15, more preferably at least 20, nucleotides that encode the unique tail. Primers are generally 8-50 nucleotides, preferably 18-28 nucleotides. A set of primers contains two primers. When performing PCR on extracted mRNA or cDNA generated therefrom, if the mRNA or cDNA encoding a CD40 splice variant is present, multiple copies of the mRNA or cDNA will be made. If it is not present, PCR will not generate a discrete detectable product.

The PCR product, i.e., amplified DNA, may be detected by several well known means. The preferred method for detecting the presence of amplified DNA is resolving the PCR reaction material by gel electrophoresis and staining the gel with ethidium bromide in order to visual the amplified DNA if present. A size standard corresponding to the expected size of the amplified DNA is preferably run on the gel as a control.

In some instances, e.g., when an unusually small amount of RNA is recovered and only small amounts of cDNA are generated therefrom, it is desirable or necessary to perform a PCR reaction on the first PCR reaction product. In other words, if difficult to detect quantities of amplified DNA are produced by the first reaction, a second PCR can be performed to make multiple copies of DNA sequences of the first amplified DNA. A nested set of primers are used in the second PCR reaction. The nested set of primers hybridize to sequences downstream of the 5′ primer and upstream of the 3′ primer used in the first reaction.

The present invention includes oligonucleotides that are useful as primers for performing PCR methods to amplify mRNA or cDNA that encodes a CD40 splice variant.

According to the invention, diagnostic kits can be assembled that are useful to practice methods of detecting the presence of mRNA or cDNA that encodes a CD40 splice variant in samples. Such diagnostic kits include oligonucleotides that are useful as PCR primers. Preferably, the diagnostic kits include a container comprising a size marker to be run as a standard on a gel used to detect the presence of amplified DNA. The size marker is the same size as the DNA generated by the primers in the presence of the mRNA or cDNA encoding a CD40 splice variant. Additional components in some kits include buffer, positive controls, negative controls, graphics or photographs depicting positive and/or negative results and instructions for carrying out the assay.

Another method of determining whether a sample contains cells expressing a CD40 splice variant is by Northern Blot analysis of mRNA from a sample. The techniques for performing Northern blot analyses are well known by those having ordinary skill in the art and are described in Sambrook et al., (1989) Molecular Cloning: A Laboratory Manual, Cold Spring Harbor Laboratory Press, Cold Spring Harbor, N.Y. mRNA extraction, electrophoretic separation of the mRNA, blotting, probe preparation and hybridization are all well known techniques that can be routinely performed using readily available starting material.

One having ordinary skill in the art, performing routine techniques, could design probes to identify mRNA encoding a CD40 splice variant. The probe must selectively hybridize to the mRNA that encodes the CD40 splice variant. Consequently, the probe includes sequences that are complementary to the sequence encoding at least a portion of the unique tail such that the probe will only hybridize if the transcript encodes the unique tail of a CD40 splice variant. Accordingly, the a portion of sequence that encodes the unique tail must be sufficient to allow selectively hybridization. Such a portion is preferably at least 8, more preferably at least 10, more preferably at least 15, more preferably at least 20 nucleotides, more preferably at least 30 nucleotides, more preferably the entire length of coding sequence of the unique tail.

The mRNA is extracted using poly dT columns and the material is separated by electrophoresis and, e.g., transferred to nitrocellulose paper. Labeled probes made from an isolated specific fragment or fragments can be used to visualize the presence of a complementary fragment fixed to the paper.

According to the invention, diagnostic kits can be assembled which are useful to practice methods of detecting the presence of mRNA that encodes CD40 splice variant in samples by Northern blot analysis. Such diagnostic kits comprise oligonucleotides that are useful as probes for hybridizing to the mRNA. The probes may be radiolabeled. It is preferred that diagnostic kits according to the present invention include a container comprising a size marker to be run as a standard on a gel. It is preferred that diagnostic kits according to the present invention include a container comprising a positive control which will hybridize to the probe. Additional components in some kits include positive controls, negative controls, graphics or photographs depicting positive and/or negative results and instructions for carrying out the assay.

In some embodiments of the invention, the method for detecting a nucleic acid sequence that encodes a CD40 splice variants in a biological sample, includes the steps of:
(a) providing a probe including at least one of the nucleic acid sequences described above;
(b) contacting the biological sample with said probe under conditions allowing hybridization of nucleic acid sequences thereby enabling formation of hybridization complexes;
(c) detecting hybridization complexes, wherein the presence of the complex indicates the presence of nucleic acid sequence encoding the CD40 splice variant in the biological sample.

This assay typically involves obtaining total mRNA from the tissue or serum and contacting the mRNA with a nucleic acid probe. The probe is a nucleic acid molecule of at least 10 nucleotides, preferably 20 nucleotides, preferably 20-30 nucleotides or more, capable of specifically hybridizing with a sequence included within the sequence of a nucleic acid molecule encoding the CD40R variant product under hybridizing conditions, detecting the presence of mRNA hybridized to the probe, and thereby detecting the expression of variant. This assay can be used to distinguish between absence or presence of CD40 splice variant.

In addition to be being qualitative, i.e., indicating whether the transcripts are present in or absent from the sample, the method can also be quantitative, by determining the level of hybridization complexes and then calibrating said levels to determining levels of transcripts of the desired variants in the sample. Thus, the assay can be used to determine excess expression of CD40 splice variant and to monitor levels of CD40 splice variant expression during therapeutic intervention.

Both qualitative and quantitative determination methods can be used for diagnostic, prognostic and therapeutic planning purposes. By a preferred embodiment the probe is part of a nucleic acid chip used for detection purposes, i.e., the probe is a part of an array of probes each present in a known location on a solid support.

In addition, the assay may be used to compare the levels of the CD40 splice variant of the invention to the levels of the original CD40 sequence from which it has been varied or to levels of each other, which comparison may have some physiological meaning.

The nucleic acid sequences used in the above method may be a DNA sequence an RNA sequence, etc; they may be a coding or a sequence or a sequence complementary thereto (for respective detection of RNA transcripts or coding-DNA sequences). By quantifying the level of hybridization complexes and calibrating the quantified results it is possible also to detect the level of the transcripts in the sample.

Methods for detecting mutations in the region coding for the CD40 splice variants are also provided, which may be methods carried-out in a binary fashion, namely merely detecting whether there is any mismatches between the normal variant nucleic acid sequence of the invention and the one present in the sample, or carried-out by specifically detecting the nature and location of the mutation.

In some embodiments of the invention, nucleic acid molecules are used as a diagnostic for diseases resulting from inherited defective variants sequences, or diseases in which the ratio of the amount of the original CD40 sequence from which the CD40 splice variants were varied to the novel CD40 splice variant of the invention is altered. These sequences can be detected by comparing the sequences of the defective (i.e., mutant) CD40 splice variant coding region with that of a normal coding region. Association of the sequence coding for mutant CD40 splice variant products with abnormal variant products activity may be verified. In addition, sequences encoding mutant CD40 splice variants can be inserted into a suitable vector for expression in a functional assay system (e.g., colorimetric assay, complementation experiments in a variant protein deficient strain of HEK293 cells) as yet another means to verify or identify mutations. Once mutant genes have been identified, one can then screen populations of interest for carriers of the mutant gene.

Individuals carrying mutations in the nucleic acid sequences of the present invention may be detected at the DNA level by a variety of techniques. Nucleic acids used for diagnosis may be obtained from a patient's cells, e.g., from blood, urine, saliva, placenta, tissue biopsy and autopsy material. Genomic DNA may be used directly for detection or may be amplified enzymatically by using PCR (Saiki et al., Nature 324:163-166, (1986)) prior to analysis. RNA or cDNA may also be used for the same purpose. As an example, PCR primers complementary to the nucleic acid of the present invention can be used to identify and analyze mutations in the gene of the present invention. Deletions and insertions can be detected by a change in size of the amplified product in comparison to the normal genotype.

Point mutations can be identified by hybridizing amplified DNA to radiolabeled RNA of the invention or alternatively, radiolabeled antisense DNA sequences of the invention. Sequence changes at specific locations may also be revealed by nuclease protection assays, such RNase and SI protection or the chemical cleavage method (see, e.g., Cotton et al., Proc. Natl. Acad. Sci. USA, 85:4397-4401, (1985)), or by differences in melting temperatures. Molecular beacons (Kostrikis et al., Science 279:1228-1229, (1998)), hairpin-shaped, single-stranded synthetic oligo-nucleotides containing probe sequences that are complementary to the nucleic acid of the present invention, may also be used to detect point mutations or other sequence changes as well as monitor expression levels of variant product. Such diagnostics would be particularly useful for prenatal testing.

Another method for detecting mutations uses two DNA probes which are designed to hybridize to adjacent regions of a target, with abutting bases, where the region of known or suspected mutation(s) is at or near the abutting bases. The two probes may be joined at the abutting bases, e.g., in the presence of a ligase enzyme, but only if both probes are correctly base paired in the region of probe junction. The presence or absence of mutations is then detectable by the presence or absence of ligated probe.

Also suitable for detecting mutations in the CD40 splice variants products coding sequences are oligonucleotide array methods based on sequencing by hybridization (SBH), as described in, e.g., U.S. Pat. No. 5,547,839. In a typical method, the DNA target analyte is hybridized with an array of oligonucleotides formed on a microchip. The sequence of the target can then be read from the pattern of target binding to the array.

Transgenic Animals

According to another aspect of the invention, transgenic animals, particularly transgenic mice, are generated. In some embodiments, the transgenic animals according to the invention contain a nucleic acid molecule which encodes a CD40 splice variant protein. Such transgenic mice may be used as animal models for studying overexpression of the CD40 splice variant protein and for use in drug evaluation and discovery efforts to find compounds effective to inhibit or modulate its activity. One having ordinary skill in the art using standard techniques, such as those taught in U.S. Pat. No. 4,873,191 issued Oct. 10, 1989 Wagner and U.S. Pat. No. 4,736,866 issued Apr. 12, 1988 to Leder, both of which are incorporated herein by reference, can produce transgenic animals which produce the CD40 splice variant protein and use the animals in drug evaluation and discovery projects.

Drug Discovery

The CD40 splice variants may also be used for screening or constructing pharmaceuticals with improved specificity. Targeting pharmaceuticals to specific tissues (which express one variant), or targeting them against one condition (in which a particular variant is expressed) may be aided by the variants of the invention which enable screening or construction pharmaceuticals with improved tissue, or condition specificity.

Sequences

The following is a partial list of nucleic acid and amino acid sequences disclosed in the application:

Sequence Listing

SEQ ID NO:1
>R49883_7|Length: 1088|Exon numbers: 1, 2, 3, 4, 5, 8, 9,
10, 11
ggctggggcaggggagtcagcagaggcctcgctcgggcgcccagtggtcctgccgcctgg
tctcacctcgccatggttcgtctgcctctgcagtgcgtcctctggggctgcttgctgacc
gctgtccatccagaaccacccactgcatgcagagaaaaacagtacctaataaacagtcag
tgctgttctttgtgccagccaggacagaaactggtgagtgactgcacagagttcactgaa
acggaatgccttccttgcggtgaaagcgaattcctagacacctggaacagagagacacac
tgccaccagcacaaatactgcgaccccaacctagggcttcgggtccagcagaagggcacc
tcagaaacagacaccatctgcacctgtgaagaaggctggcactgtacgagtgaggcctgt
gagagctgtgtcctgcaccgctcatgctcgcccggctttggggtcaagcagattgctaca
ggggtttctgataccatctgcgagccctgcccagtcggcttcttctccaatgtgtcatct
gctttcgaaaaatgtcacccttggacaagctgtgagaccaaagacctggttgtgcaacag
gcaggcacaaacaagactgatgttgtctgtggtgagtcctggacaatgggccctggagaa
agcctaggaaggtgggaactgaagggggagatgaggcacacaggaacactggatgggaaa
aaggggaggggaggcagtttgggggtgtggtatcacagctctgccacttatcttgggagt
ctgggcaaatcacttcccctctcttagcctcagtttcttcatctgtaaaatgggatgata
acagcacttccttagtaggttttgattttagagtgagaaggttggcctacagtaaagatc
agataatgtaaatcagtgaaaaaggtcaggggtaagaaaattacattctctttacctaac
gctaaatgaccagttaatgggtgcagcacaccaacatggtacatgtatacatatgtaaca
aacctgcacattatgcacatgtaccctaaagcttaaagtataataataataaaatttaaa
aaaacgaa
SEQ ID NO:2
>195465|Length: 244|Encoding transcript: 7
MVRLPLQCVLWGCLLTAVHPEPPTACREKQYLINSQCCSLCQPGQKLVSDCTEFTETECL
PCGESEFLDTWNRETHCHQHKYCDPNLG
LRVQQKGTSETDTICTCEEGWHCTSEACESCVLHRSCSPGFGVKQIATGVSDTICEPCPV
GFFSNVSSAFEKCHPWTSCETKDLVVQQAGTNKTDVVCGESWTMGPGESLGRWELKGEM
RHTGTLDGKKGRGGSLGVWYHSSATYLGSLGKSLPLS
SEQ ID NO:3
>R49883_15|Length: 1813|Exon numbers: 1, 2, 3, 4, 5, 8,
13, 16, 17, 19, 20, 22, 23
ggctggggcaggggagtcagcagaggcctcgctcgggcgcccagtggtcctgccgcctgg
tctcacctcgccatggttcgtctgcctctgcagtgcgtcctctggggctgcttgctgacc
gctgtccatccagaaccacccactgcatgcagagaaaaacagtacctaataaacagtcag
tgctgttctttgtgccagccaggacagaaactggtgagtgactgcacagagttcactgaa
acggaatgccttccttgcggtgaaagcgaattcctagacacctggaacagagagacacac
tgccaccagcacaaatactgcgaccccaacctagggcttcgggtccagcagaagggcacc
tcagaaacagacaccatctgcacctgtgaagaaggctggcactgtacgagtgaggcctgt
gagagctgtgtcctgcaccgctcatgctcgcccggctttggggtcaagcagattgctaca
ggggtttctgataccatctgcgagccctgcccagtcggcttcttctccaatgtgtcatct
gctttcgaaaaatgtcacccttggacaagctgtgagaccaaagacctggttgtgcaacag
gcaggcacaaacaagactgatgttgtctgtgggctgggactagaatgaggtgagcaaggc
acttgccctcgggcgcaatatttaagaaggtgccataaaagtgtagtaatcaaggtcccc
aggatcggctgagagccctggtggtgatccccatcatcttcgggatcctgtttgccatcc
tcttggtgctggtctttatcaaaaaggtggccaagaagccaaccaataaggccccccacc
ccaagcaggaaccccaggagatcaattttcccgacgatcttcctggctccaacactgctg
ctccagtgcaggagactttacatggatgccaaccggtcacccaggaggatggcaaagaga
gtcgcatctcagtgcaggagagacagtgaggctgcacccacccaggagtgtggccacgtg
ggcaaacaggcagttggccagagagcctggtgctgctgctgctgtggcgtgagggtgagg
ggctggcactgactgggcatagctccccgcttctgcctgcacccctgcagtttgagacag
gagacctggcactggatgcagaaacagttcaccttgaagaacctctcacttcaccctgga
gcccatccagtctcccaacttgtattaaagacagaggcagaagtttggtggtggtggtgt
tggggtatggtttagtaatatccaccagaccttccgatccagcagtttggtgcccagaga
ggcatcatggtggcttccctgcgcccaggaagccatatacacagatgcccattgcagcat
tgtttgtgatagtgaacaactggaagctgcttaactgtccatcagcaggagactggctaa
ataaaattagaatatatttatacaaacagaatctcaaaaacactgttgagtaaggaaaaa
aaggcatgctgctgaatgatgggtatggaactttttaaaaaagtacatgcttttatgtat
gtatattgcctatggatatatgtataaatacaatatgcatcatatattgatataacaagg
gttctggaagggtacacagaaaacccacagctcgaagagtggtgacgtctggggtgggga
agaagggtctgggggagggttggttaaagggagatttggctttcccataatgcttcatca
tttttcccaaaaggagagtgaattcacataatgcttatgtaattaaaaaatcatcaaaca
tgtaaaaagaaaa
SEQ ID NO:4
>195468|Length: 191|Encoding transcript: 15, 17, 16
MVRLPLQCVLWGCLLTAVHPEPPTACREKQYLINSQCCSLCQPGQKLVSDCTEFTETECL
PCGESEFLDTWNRETHCHQHKYCDPNLGLRVQQKGTSETDTICTCEEGWHCTSEACESC
VLHRSCSPGFGVKQIATGVSDTICEPCPVGFFSNVSSAFEKCHPWTSCETKDLVVQQAGT
NKTDVVCGLGLE
SEQ ID NO:5
>R49883_18|Length: 1770|Exon numbers: 1, 2, 3, 4, 5, 8,
9, 16, 17, 19, 20, 22, 23
ggctggggcaggggagtcagcagaggcctcgctcgggcgcccagtggtcctgccgcctgg
tctcacctcgccatggttcgtctgcctctgcagtgcgtcctctggggctgcttgctgacc
gctgtccatccagaaccacccactgcatgcagagaaaaacagtacctaataaacagtcag
tgctgttctttgtgccagccaggacagaaactggtgagtgactgcacagagttcactgaa
acggaatgccttccttgcggtgaaagcgaattcctagacacctggaacagagagacacac
tgccaccagcacaaatactgcgaccccaacctagggcttcgggtccagcagaagggcacc
tcagaaacagacaccatctgcacctgtgaagaaggctggcactgtacgagtgaggcctgt
gagagctgtgtcctgcaccgctcatgctcgcccggctttggggtcaagcagattgctaca
ggggtttctgataccatctgcgagccctgcccagtcggcttcttctccaatgtgtcatct
gctttcgaaaaatgtcacccttggacaagctgtgagaccaaagacctggttgtgcaacag
gcaggcacaaacaagactgatgttgtctgtggtgagtcctggacaatgggccctggagaa
agcctaggaaggtccccaggatcggctgagagccctggtggtgatccccatcatcttcgg
gatcctgtttgccatcctcttggtgctggtctttatcaaaaaggtggccaagaagccaac
caataaggccccccaccccaagcaggaaccccaggagatcaattttcccgacgatcttcc
tggctccaacactgctgctccagtgcaggagactttacatggatgccaaccggtcaccca
ggaggatggcaaagagagtcgcatctcagtgcaggagagacagtgaggctgcacccaccc
aggagtgtggccacgtgggcaaacaggcagttggccagagagcctggtgctgctgctgct
gtggcgtgagggtgaggggctggcactgactgggcatagctccccgcttctgcctgcacc
cctgcagtttgagacaggagacctggcactggatgcagaaacagttcaccttgaagaacc
tctcacttcaccctggagcccatccagtctcccaacttgtattaaagacagaggcagaag
tttggtggtggtggtgttggggtatggtttagtaatatccaccagaccttccgatccagc
agtttggtgcccagagaggcatcatggtggcttccctgcgcccaggaagccatatacaca
gatgcccattgcagcattgtttgtgatagtgaacaactggaagctgcttaactgtccatc
agcaggagactggctaaataaaattagaatatatttatacaaacagaatctcaaaaacac
tgttgagtaaggaaaaaaaggcatgctgctgaatgatgggtatggaactttttaaaaaag
tacatgcttttatgtatgtatattgcctatggatatatgtataaatacaatatgcatcat
atattgatataacaagggttctggaagggtacacagaaaacccacagctcgaagagtggt
gacgtctggggtggggaagaagggtctgggggagggttggttaaagggagatttggcttt
cccataatgcttcatcatttttcccaaaaggagagtgaattcacataatgcttatgtaat
taaaaaatcatcaaacatgtaaaaagaaaa
SEQ ID NO:6
>195469|Length: 237|Encoding transcript: 20, 18, 19
MVRLPLQCVLWGCLLTAVHPEPPTACREKQYLINSQCCSLCQPGQKLVSDCTEFTETECL
PCGESEFLDTWNRETHCHQHKYCDPNLGLRVQQKGTSETDTICTCEEGWHCTSEACESC
VLHRSCSPGFGVKQIATGVSDTICEPCPVGFFSNVSSAFEKCHPWTSCETKDLVVQQAGT
NKTDVVCGESWTMGPGESLGRSPGSAESPGGDPHHLRDPVCHPLGAGLYQKGGQEANQ
SEQ ID NO:7
MVRLPLQCVLWGCLLTAVHPEPPTACREKQYLINSQCCSLCQPGQKLVSD
CTEFTETECLPCGESEFLDTWNRETHCHQHKYCDPNLGLRVQQKGTSETD
TICTCEEGWHCTSEACESCV LHRSCSPGFG VKQIA VRPKTWLCNRQAQTRLMLSVV
SEQ ID NO:8
MVRLPLQCVLWGCLLTAVHPEPPTACREKQYLINSQCCSLCQPGQKLVSDCTEFTETECL
PCGESEFLDTWNRETHCHQHKYCDPNLGLRVQQKGTSETDTICTCEEGWHCTSEACESC
VLHRSCSPGFGVKQIATGVSDTICEPCPVGFFSNVSSAFEKCHPWTSCETKDLVVQQAGT
NKTDVVCGESWTMGPGESLG RSPGSAESPGGDPHHLRDPVCHPLGAGLYQ
SEQ ID NO:9
ESRGEWPCQVFGKQGTGERLRLHAGTLTGIGVRPRGSLXYSTL
SEQ ID NO:10
MVSLPRLCALWGCLLTAVHLGQCVTCSDKQYLHDGQCCDLCQPGSRLTSH
CTALEKTQCHPCDSGEFSAQWNREIRCHQHRHCEPNQGLRVKKEGTAESDTV
CTCKEGQHCTSKDCEACAQHTPCIPGFGVMEMAVRIRTWRSYRKERVRLMSSVVPRIG
SEQ ID NO:25
CELKGEMRHTGTLDGKKGRG
SEQ ID NO:26:
CGESWTMGPGESLGR
SEQ ID NO:27
GGGG ACA AGT TTG TAC AAA AAA GCA GGC TNN
SEQ ID NO:28
GGGG ACA AGT TTG TAC AAA AAA GCA GGC Tcc
atg gtt cgt ctg cct ctg cag
SEQ ID NO:29
GGGG AC CAC TTT GTA CAA GAA AGC TGG GTN
SEQ ID NO:30
GGGG AC CAC TTT GTA CAA GAA AGC TGG Gta
cta aga gag ggg aag tga ttt
SEQ ID NO:31
GGGG AC CAC TTT GTA CAA GAA AGC TGG Gta
tta ttg gtt ggc ttc ttg gcc
SEQ ID NO:32
CGCGTTAACGCTAGCATGGAT
SEQ ID NO:33
CACAGAGTTTTAGAGACTACAAT
SEQ ID NO:34
CACCATCTGCACCTGTGAAG
SEQ ID NO:35
NNNN AgATC TTA AgA gAg ggg AAg TgA TTT
SEQ ID NO:36
NNNN AgATC TTA TTC TAg TCC CAg CCC AC
SEQ ID NO:37
NNNN AgATC TTA TTg gTT ggC TTC TTg gC

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Sf-9 cells are infected with sCD40 expressing baculovirus (Ac-sCD40) including a coding sequence that encodes a protein with an amino acid sequence of a CD40 splice variant of the invention. The cells are grown in 28° C. at continuous shaking (90 rpm). At 60 hours post infection (hpi) the medium is collected and cells are separated from the medium by centrifugation at 5000 RPM for 5 minutes. 10 ml medium is separated using cation exchange chromatography with SP-Sepharose column. The Column is equilibrated with PBS pH-6.5 and following loading of the sample on the column the column is washed with PBS to elute the unbound proteins (flow through fraction). Elution is done with increasing concentration of NaCl at flow rate of 2 ml/min (5% NaCl/min).

The different fractions are subjected to SDS-PAGE electrophoresis and to western blotting using anti mCD40 antibody.

Sf-9 cells are infected with sCD40 expressing baculovirus (Ac-sCD40) at MOI of 2. The cells are grown at 28° C. at continuous shaking (90 rpm) and 1-ml samples are collected at 24, 48 and 60 hours post infection (hpi). Following centrifugation, the cell pellet is lysed with lysis buffer (50 mM Tris pH 7.5, 1% triton X100, and protease inhibitor cocktail) at 4° C. for 30 min and sonicated for 30 seconds. The sample is centrifuged for 10 minutes at 14000 RPM and the sup is designated Pellet. 40 μl of the pellet preparation and of the medium (Designated Medium) are supplemented with sample buffer and electrophoresed on a 15% SDS-PAGE. Following electrophoresis, the gel is subjected to a semi dry protein transfer onto a nitrocellulose membrane. The membrane is incubated with anti mCD40 antibody for 2 hours and with secondary anti rabbit antibody for an additional 1 hour.

Detection of the signal is done using a commercial western blot detection kit.

### Example 2

### Production of Polyclonal Antibodies Specific to CD40 Variants NJ1, NJ2 and NJ3

Rabbits were immunized with two peptides, both of which were KLH conjugated and 95% purified peptides: CELKGEMRHTGTLDGKKGRG (SEQ ID NO: 25), a sequence taken from the unique tail of the CD40 NJ1 splice variant, and CGESWTMGPGESLGR (SEQ ID NO: 26), a sequence taken from the unique tail of the CD40 NJ3 splice variant.

The anti-NJ3 antibodies were then purified from rabbit serum by ammonium sulfate precipitation. Briefly, a saturated solution of ammonium sulfate was prepared by adding 380 gr to 500 ml water and boiling the solution. The serum was thawed and centrifuged at 10,000 rpm, 4° C. for 5 min. One vol. PBS was added to each vol. serum, and stirred at 4° C.

One volume of saturated ammonium sulfate was then added under stirring for at least 2 hours on ice. The solution was centrifuged 15 min. at 10,000 rpm at 4° C. to precipitate IgG. The pellet was resuspended in 5 ml PBS and dialyzed overnight at 4° C. against PBS+0.05% azide. The precipitated serum was filtered with a 0.45 μm filter.

Affinity purification was then performed with the peptide against which the respective antibodies were raised as described above, SEQ ID NOs: 25 or 26 for the variants NJ1 or NJ3, respectively, in an immunoaffinity column, linked to sulfolink beads (Pierce # 20401). The column was prepared according to manufacturer's instructions. The serum to be purified was mixed with sulfolink beads and incubated under gentle shaking (1 h at room temperature and 2 h at 4° C.), after which the beads were packed into a column.

The column was washed with TRIS 100 mM, followed by binding buffer containing 0.5M NaCl. The IgG was eluted by applying elution buffer: 0.1M Glycine pH3 (fraction size: 0.5 ml), followed by phosphate buffer 100 mM pH 11 to elute another fraction of IgG. In order to neutralize acidic or basic pH, 1/10 volume TRIS 1M pH8 was added to collecting tubes before addition of elution buffer to the column. The antibodies were dialyzed overnight against a buffer of PBS and 0.025% azide, and then frozen for storage.

The anti-NJ1 serum was used without purification.

The results of the Western blot are shown below with regard to Example 4.

### Example 3

### Cloning and Purification of the CD40 NJ1 and NJ3 Variants

This Example describes cloning of both splice variants in bacteria or by using the baculovirus system; the latter expressed proteins were also purified.

Cloning into Bacteria

mRNA from the K562 cell line was isolated and subjected to reverse transcription using random hexamer primer mix and Superscript™, followed by a treatment of DNAse I.

The NJ1 and NJ3 splice variant cloning fragments were prepared by PCR amplification using TaKaRa Hot-Start Ex-Taq™ under the following conditions: 2.5 μl—Ex-Taq X10 buffer; 5 μl—cDNA; 2 μl—dNTPs (2.5 mM each); 0.5 μl—Ex-Taq enzyme; 14 μl—H₂O; and 0.5 μl—of each primer in a total reaction volume of 25 μl; with a reaction program of 5 minutes in 95° C.; 40 cycles of: 30 seconds at 94° C., 45 seconds at 68° C., 60 seconds at 72° C. and 10 minutes at 72° C.

The following primers, comprising specific sequences of the nucleotide sequence corresponding to the splice variants and Gateway™ BP recombination tails were used:

| | |
|---|---|
| 1. Gateway forward attB1 primer: | |
| 5′-GGGG ACA AGT TTG TAC AAA AAA GCA | (SEQ ID NO:27) |
| GGC TNN | |
| 2. NJ1/NJ3 forward primer: | |
| 5′-GGGG ACA AGT TTG TAC AAA AAA GCA | (SEQ ID NO:28) |
| GGC Tcc atg gtt cgt ctg cct ctg | |
| cag-3′ | |
| 3. Gateway reverse attB2 primer: | |
| 5′-GGGG AC CAC TTT GTA CAA GAA AGC | (SEQ ID NO:29) |
| TGG GTN | |
| 4. NJ1 reverse primer: | |
| 5′-GGGG AC CAC TTT GTA CAA GAA AGC | (SEQ ID NO:30) |
| TGG Gta cta aga gag ggg aag tga ttt | |
| 5. NJ3 reverse primer: | |
| 5′-GGGG AC CAC TTT GTA CAA GAA AGC | (SEQ ID NO:31) |
| TGG Gta tta ttg gtt ggc ttc ttg gcc | |

Reaction with primer set 2+4 gave the NJ1 splice variant.

Reaction with primer set 2+5 gave the NJ3 splice variant.

PCR products were run in a 2% agarose gel, TBEX1 solution at 150V, and extracted from gel using QiaQuick™ gel extraction kit (Qiagen™). The extracted DNA products were sequenced by direct sequencing using Gateway primers (Forward=primer number 1 above; and Reverse=primer number 3 above). Error-free inserts were introduced into Gateway™ entry clone (Invitrogen™) by a BP clonase reaction (according to the manufacturer protocol), and DH5α competent bacteria were transfected with the resulted clones using the following protocol:

5 μl of each BP reaction product were mixed with freshly thawed 50 μl of competent DH5α cells. The mix was incubated on ice for 30 minutes and then exposed to Heat-Shock at 42° C. for 30 seconds. 450 μl of LB was added to each tube, and the tubes were incubated at 37° C. in a shaker for 1 hour.

From each transfection solution, both 50 μl and 150 μl were plated on selective LB plates containing 50 μg/ml Kanamycin. The plates were incubated at 37° C. overnight.

Ten colonies from each transcript clone that grew on the selective plates were taken for further analysis by re-plating on a selective plate and by PCR. PCR was performed using primers specific to the vector (pDONR), located upstream and downstream to the insert:

| | |
|---|---|
| pDONR-Forward: | |
| 5′-CGCGTTAACGCTAGCATGGAT-3′. | (SEQ ID NO:32) |
| pDONR-Reverse: | |
| 5′-CACAGAGTTTTAGAGACTACAAT-3′. | (SEQ ID NO:33) |

PCR products were extracted and sequenced as above, using the Gateway primers (SEQ ID NO: 27 and SEQ ID NO:29).

Colonies containing an error free insert (no mutations within the ORF), were grown overnight in 2 ml of LB+50 ng Kanamycin at 37° C. Plasmids were obtained from bacterial colonies using Qiaprep™ spin miniprep kit (Qiagen). Plasmid inserts were transferred into pDEST destination vectors (Gateway™—Invitrogen) according to the manufacturer protocol. Both constructs were transferred into pDEST26™. Accurate cloning was verified by sequencing the clones' inserts.

Cloning of NJ 1 and NJ3 in Baculo Vectors

For the construction of the pTen21-NJ Non-Fused vectors, one of the previous CD40 non fused plasmids (pTen21-CD40 wtEC or pTen21-CD40_Skip6; see U.S. Pat. No. 6,720,182) was used to replace the fragment between StuI site and the End of the gene (Replacement of the StuI-BglII fragment).

The primers which can be used for the PCR amplification of the 3 fragments are:

5′-CACCATCTGCACCTGTGAAG-3′ (SEQ ID NO: 34): Internal Stu40 primer to be used in conjunction with:

NJ1nf3′:

(For NJ1; SEQ ID NO:35)

5′-NNNN AgATC TTA AgA gAg ggg AAg TgA TTT-3′
NJ2nf3′:

(For NJ2; SEQ ID NO:36)

5′-NNNN AgATC TTA TTC TAg TCC CAg CCC AC-3′
NJ3nf3′:

(For NJ3; SEQ ID NO:37)

5′-NNNN AgATC TTA TTg gTT ggC TTC TTg gC-3′

A TAA stop codon was used for the three constructs as it is certainly the most efficient in insect cells. The BglII site is underlined.

NJ1 and NJ3 Purification Using Immunoaffinity

Proteins expressed by using the baculovirus system were then purified by immunoaffinity as described in greater detail below. The antibody used was G28-5, which is described by Ledbetter et al, J. Immunol, 1389:788-794 (1987), and is available as ascites fluid from ATCC catalog number HB-9110.

Coupling Antibodies to Protein G

Buffers:

0.2 M Borax-borate buffer pH 9.0 (Merck and Sigma)
20 mM Dimethylpimelimidate.HCl (DMP)
0.2 M ethanolamine-HCl pH 8.0 (Sigma)
   Other Materials:
Protein G Sepharose 4 Fast-Flow (Amersham)
Bio-Rad Bio-spin (Bio-Rad)

5-ml protein G beads were washed with water and then with 10-column volume (CV) borate buffer prior to being transferred to a mini column. 2.7 ml ascites fluid (10 mg IgG1) was added to the beads and mixed 2 h at room temperature on a roller. Unbound material was collected to check binding efficiency and the beads were washed with 10 CV borate buffer. 4 CV borate buffer was added to the beads before addition of 104 mg dimethylpimelimidate (DMP) to a final concentration of 20 mM. Beads were incubated on a roller for 30 min at room temperature. The reaction was stopped by washing the beads with 2 CV of 0.2 M ethanolamine pH 8.2 twice. Beads were resuspended in 1 CV 0.2 M ethanolamine pH 8.2 and incubated on roller for 2 hr. at room temperature. Finally, beads were washed with 10 CV PBS and stored in PBS-0.05% sodium azide at 4° C.

Purification of NJ1, NJ3 and Mock by Immunoaffinity

Buffers:

PBS (Biological industries)
Glycine-HCl 100 mM, pH 2.8
TRIS 1M, pH 8
   Protocol:

Baculovirus supernatants were thawed and centrifuged at 12000 rpm for 15 min. at 4° C. NJ1, NJ3 and Mock supernatants were loaded on the protein G-G28-5 column described above using FPLC at 0.5 mL/min. The beads were washed with PBS until OD was 0.01 mAu. Elution was preformed by 0.1M glycine pH 2.8. pH was fixed by 10% Tris 1M pH 8.0.

The purified proteins were then examined by gel electrophoresis, followed by Coomassie staining (described in greater detail below with regard to Example 4), which revealed highly purified proteins with only one major band at the expected molecular weight and a few faint minor bands; mock transfected cells showed only two very faint bands that were barely visible (data not shown).

### Example 4

### Recognition of CD40 NJ1 and NJ3 Variants by Specific Anti-CD40 Antibody

This Example describes Western blots that were performed by using the anti-NJ1 and anti-NJ3 antibodies (preparation of these antibodies is described above with regard to Example 2). N-16 antibody was used as a control. This is a polyclonal rabbit antibody from Santa Cruz (Cat num. Sc-974).

SDS-PAGE was performed as follows. The proteins (either non-purified bacterial preparations for the Western blot, or purified preparations as described with regard to Example 3) were resuspended in 30 μl 1× SDS-sample buffer containing 50 mM DTT (crude preparation). Following warming for 10 min and subsequent centrifugation, samples were loaded on Nu-PAGE gel buffer system (In-Vitrogen).

Following electrophoresis, for performing Western blots, gels were washed with cold transfer buffer for 15 min and taken for transfer to Nitrocellulose membranes for 60 min at 30 V using In-Vitrogen's transfer buffer and X-Cell II blot module. Following transfer, blots were blocked with TBS-5% skim milk (0.3% protein, 0.04% Tween-20) for at least 60 min. at room temperature or overnight at 4° C. Following blocking, blots were incubated with antibodies (either the previously described anti-splice variant antibodies or a commercially available N-16 antibody) at ˜1 μg/ml for 1-3 hrs, washed with 0.05% Tween-20 in TBS, incubated with respective peroxidase-conjugated antibodies, washed with TBS-Tween-20 solution, followed by ECL.

Other gels underwent Coomassie staining when used for purified proteins (see Example 3 for a description).

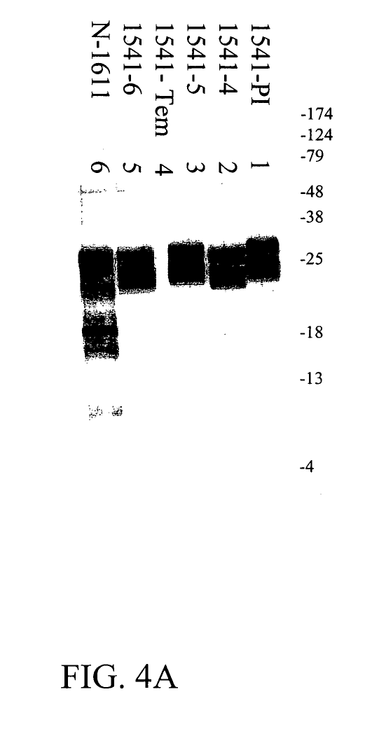

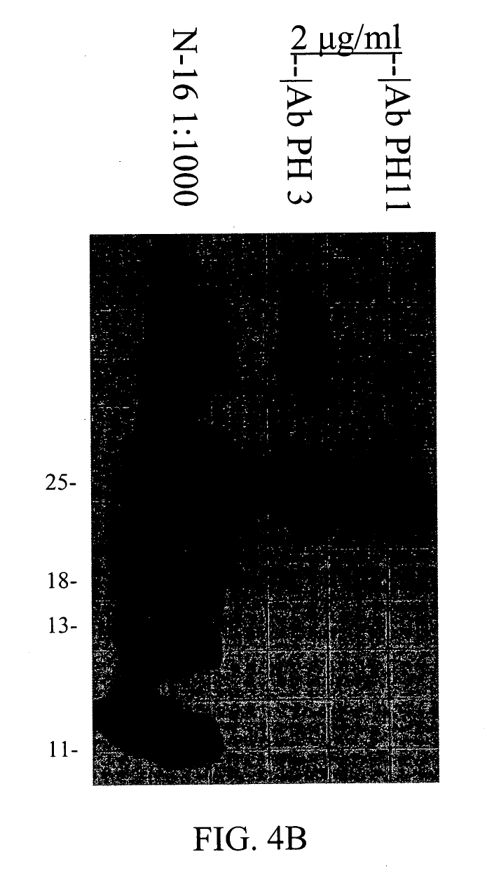

The results are shown in FIGS. 4A (anti-NJ1 antibodies) and 4B (anti-NJ3 antibodies). Briefly, FIG. 4A shows the control antibody N-16 (Santa Cruz) in the left lane, followed by anti-NJ1 antibody in the right lane. Lane 1 in FIG. 4A is a negative control, using the serum of the preimmune rabbit. Lanes 2-5 are serum samples derived from various bleeds of the immunized rabbit. The anti-NJ1 antibody clearly has specific binding with one significant band. Molecular weight markers are shown at the far right.

FIG. 4B shows the molecular weight marked in the far left lane, followed by the control N-16 antibody, with two lanes of anti-NJ3 antibody eluted at pH 3 (right) and at pH 11 (far right). Clearly, the anti-NJ3 shows specific binding to the NJ3 CD40 variant.

### Example 5

### Binding of the CD40 NJ1 and NJ3 Variants to the CD154-ELISA Assays

The binding capabilities of the purified CD40 NJ1 and NJ3 variants were assessed after the purification described above, by reacting these proteins with the CD154 ligand bound to 96 well ELISA plates, and quantifying the reaction using an ELISA reader.

Amine coupling of CD154 (ALX-522-015) to Nunc Peptide/Protein immobilizer plate was performed. Binding analysis of CD40 NJ1 and NJ3 variants and WT CD40 was performed on a small scale.

Protocol of Competition Process:

The ELISA plate (Maxisorp, Nunc) was coated with 1 μg/ml enhancer (Alexis) (100 μl/well) diluted in PBS, covered and incubated overnight at 4° C. with shaking. Then the plate was washed three times with 300 μl/well washing buffer (PBS-0.05% Tween20), followed by incubation for 2 h at 30° C. with shaking with 250 μl/well-blocking buffer (4% skim milk in washing buffer). After a thorough removal of the blocking solution, 100 μl CD154 (1 μg/ml blocking buffer) were added to the plate and incubated for 1.5 h at 30° C. with shaking, following by washing as described previously and the addition of 50 μl-blocking buffer to each well. 50 μl competitor (variant or WT CD40), diluted in blocking buffer at 1.6 μg/ml, were then added, mixed by pipeting three times and 50 μl of the mixture were transferred to the next dilution well. Dilutions were continued and the last remaining 50 μl were thrown away. After incubation for 30 min at 30° C. with shaking, 50 μl CD40-biotinylated (at 100 ng/ml) were added to each well. The plate was then covered and incubated for 2 h at 30° C. under shaking, and washed again. 100 μl/well streptavidin-HRP were then added. The plate was covered again, incubated for 1 h at 30° C. with shaking, followed by washing. Next, 100 μl/well TMB solution (washing and blocking buffers, mixed at ratio 1:1 a few minutes prior to addition) was added to the wells. Finally, the plate was covered to block light and incubated for 15 min at 30° C. under shaking. The resultant absorbance was read at 450 nm (reference 620 nm) after addition of 100 μl/well sulfuric acid 4N to stop the reaction.

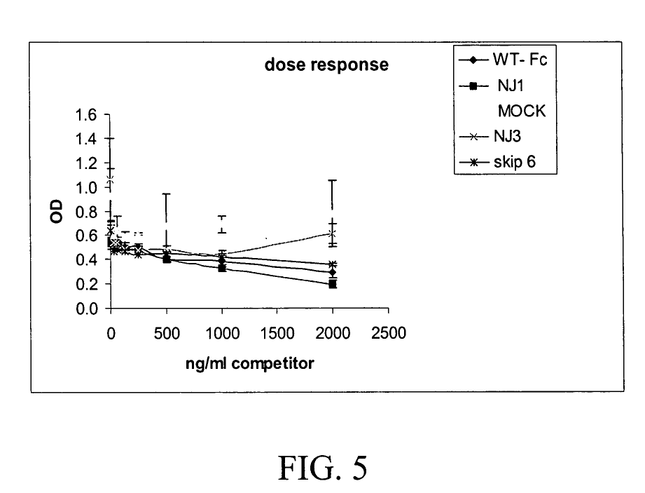

The results of the Elisa binding of the CD40 NJ1 and NJ3 variants to CD154 ligand are demonstrated in FIG. 5. According to the results the CD40 NJ1 variant retains its ability to bind CD154 ligand.

FIG. 5 shows a competition assay, in which NJ1 clearly is able to block binding of the biotinylated wild type CD40. The positive control of WT CD40 (non-labeled) also blocks binding, while the negative control of mock shows some variability but basically does not block binding. NJ3 also showed a clear ability to block binding; the last point (in which binding of the biotinylated WT CD40 seems to increase) has large error bars and appears to be an artifact.

It should be noted that another ELISA experiment performed, which was similar to the above-described ELISA experiment except that each variant CD40 was bound to the plate, and biotinylated ligand CD154 was added to examine binding to CD40. A dose-response assay was performed. In this experiment, the CD40 variant NJ1 showed clear dose-dependent binding, while the CD40 variant NJ3 did not (results not shown).

### Example 6

### FACS Analysis of the CD40 NJ1 and NJ3 Variants Binding to Mouse Fibroblasts Expressing Human CD154

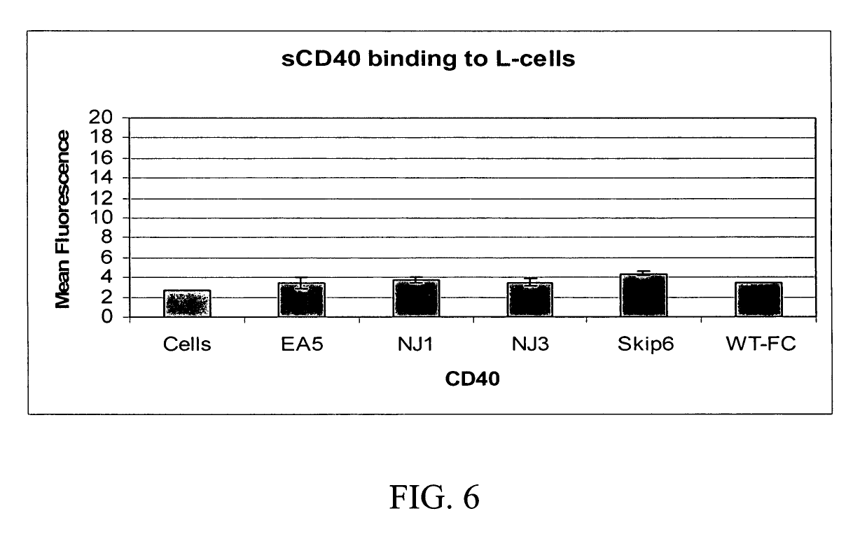

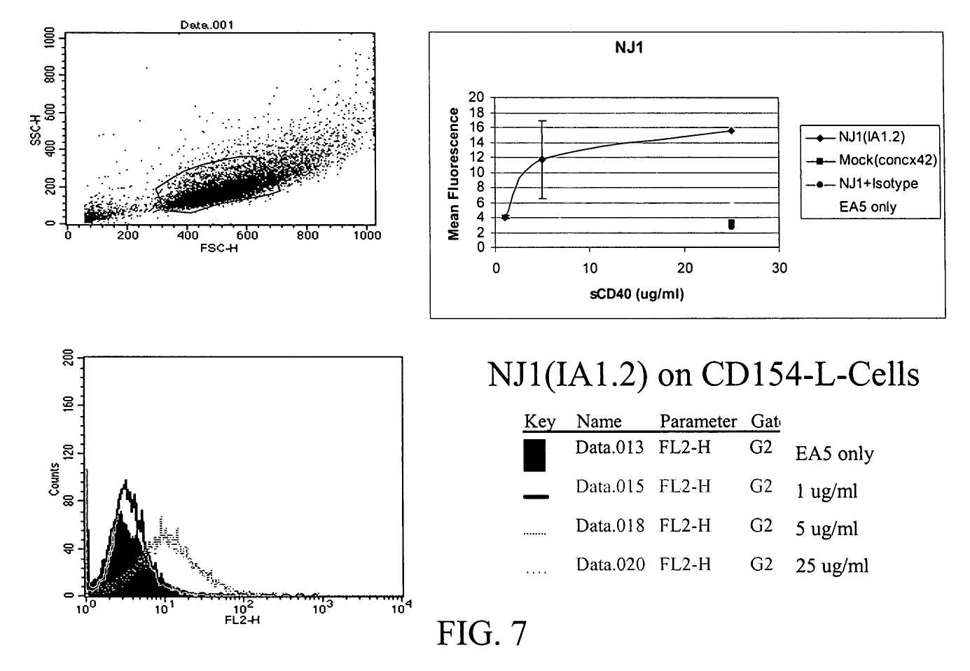

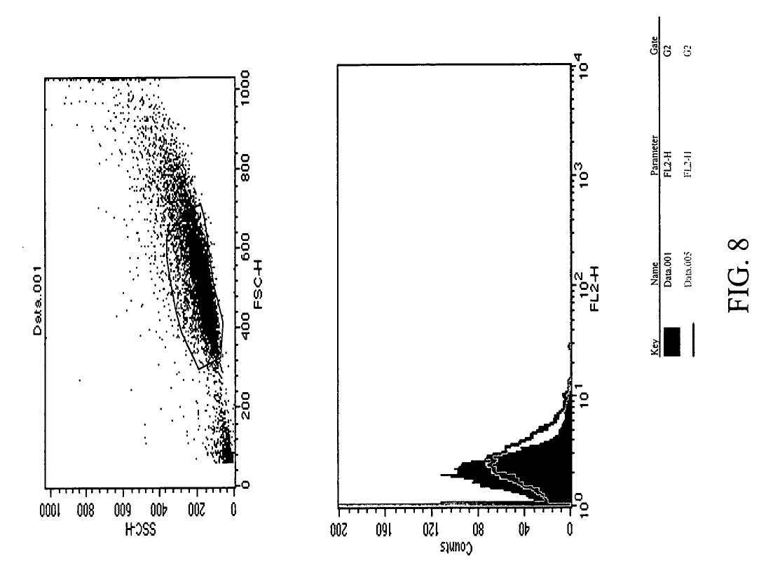

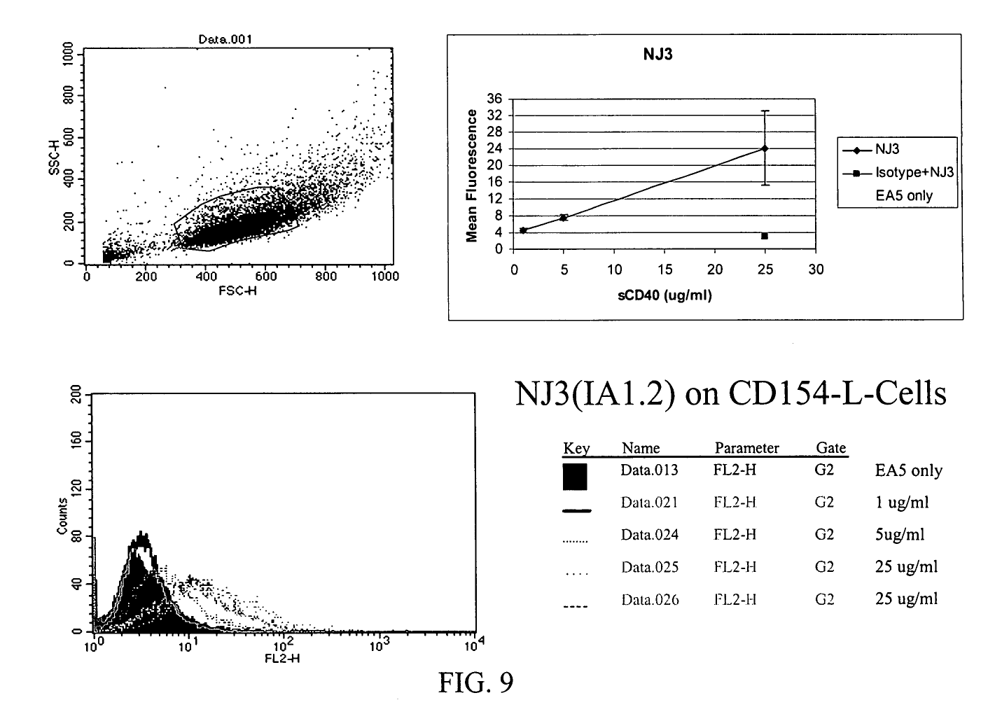

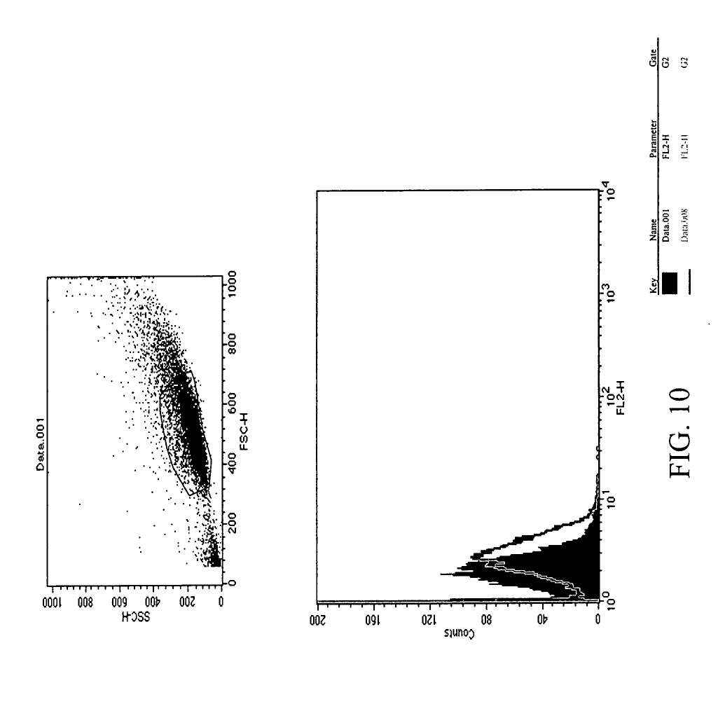

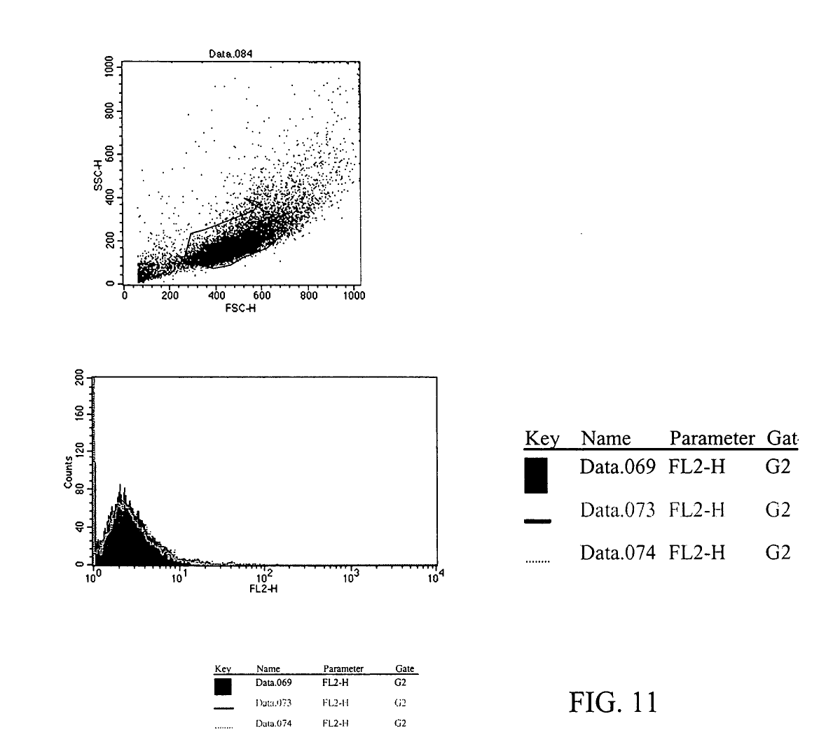

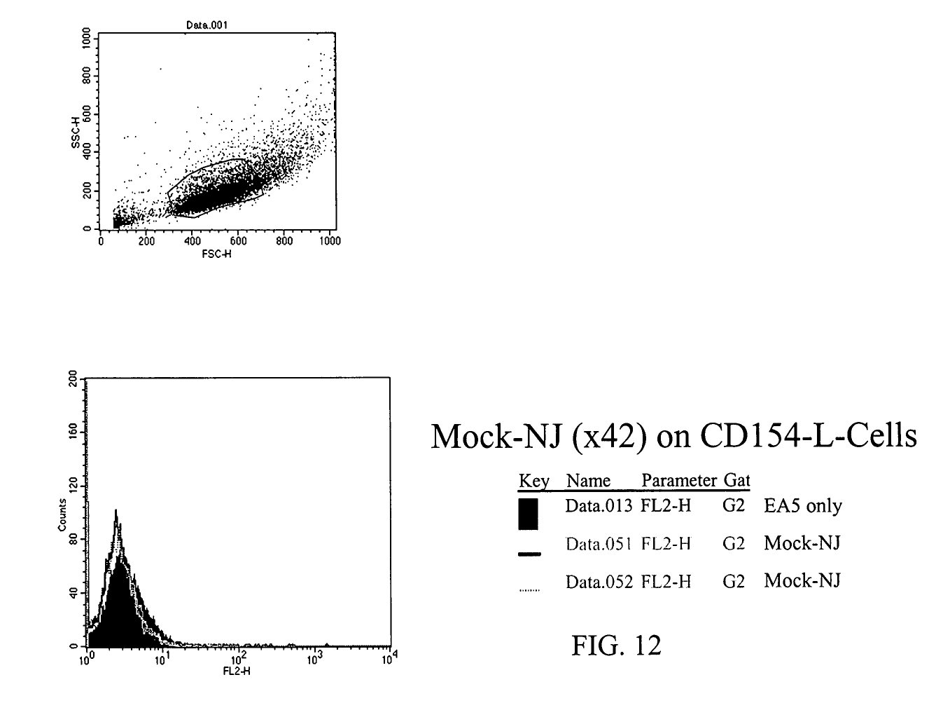

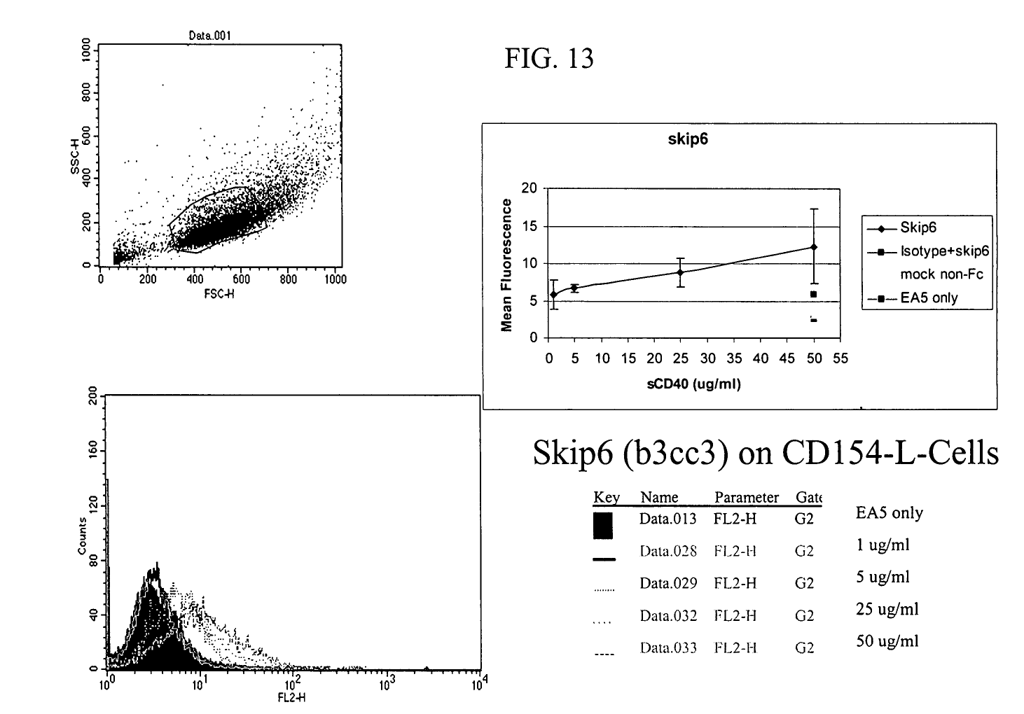

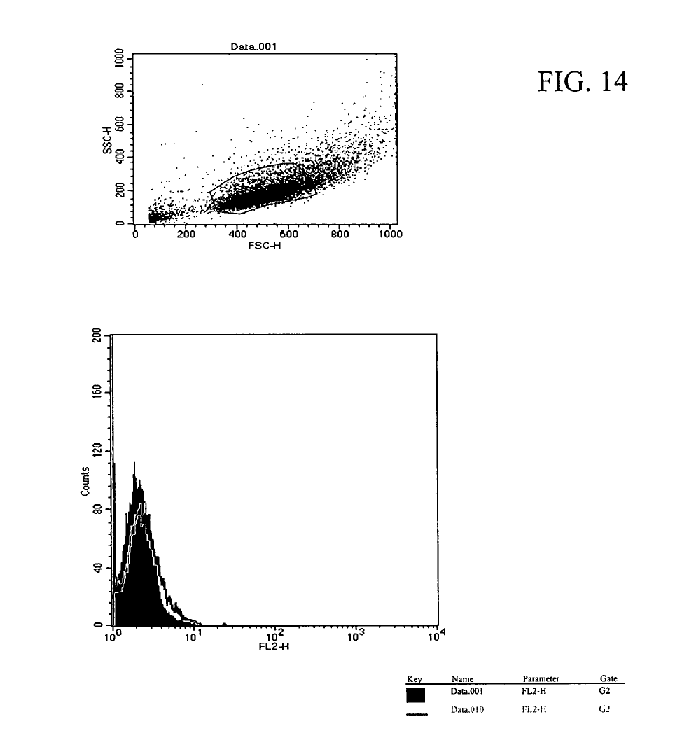

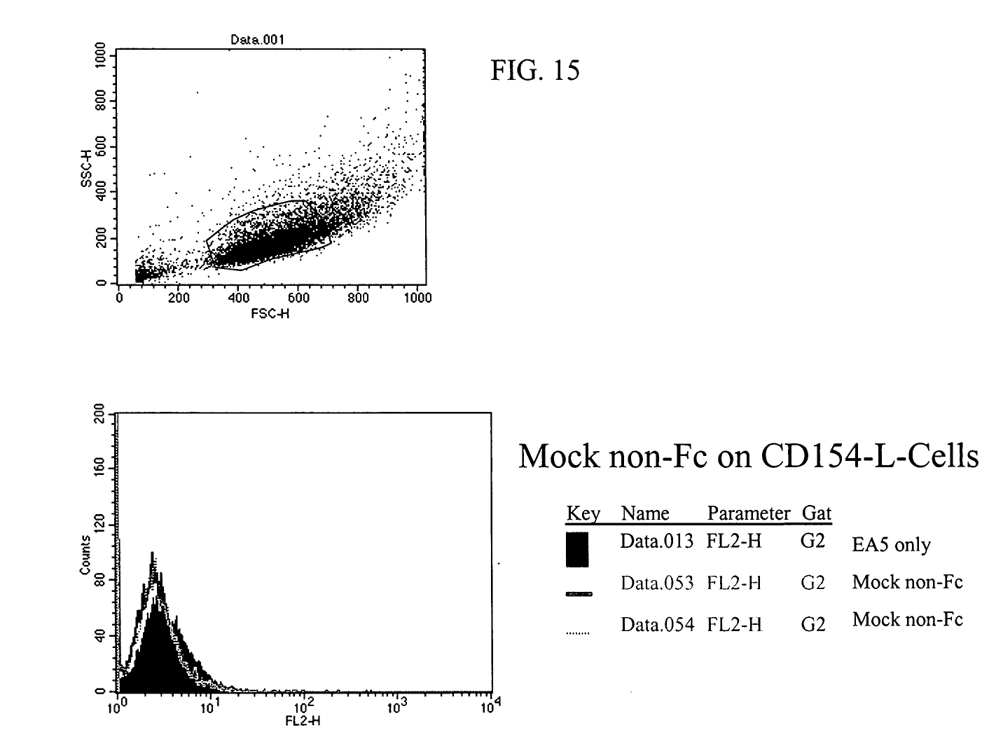

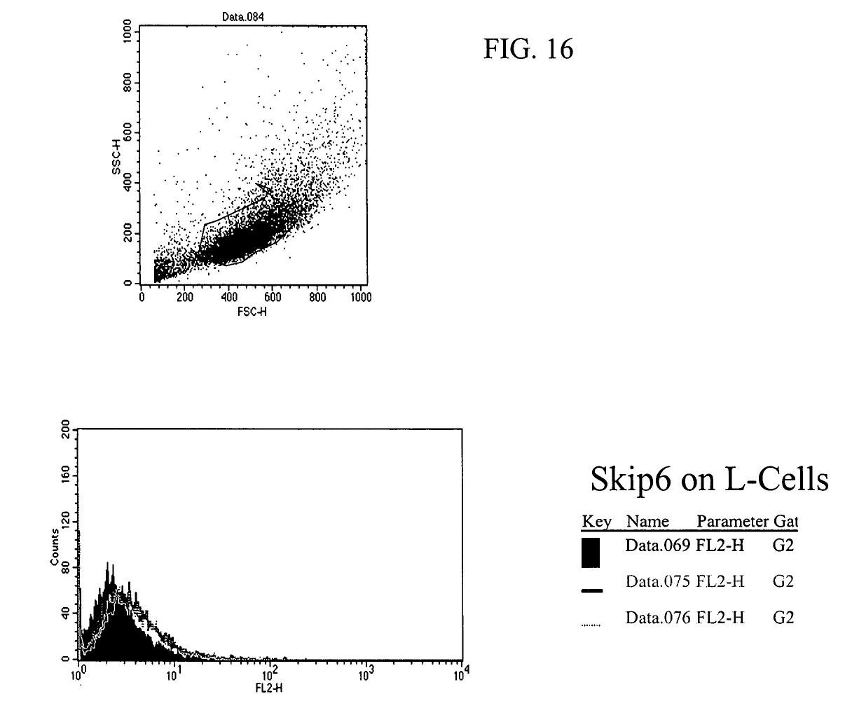

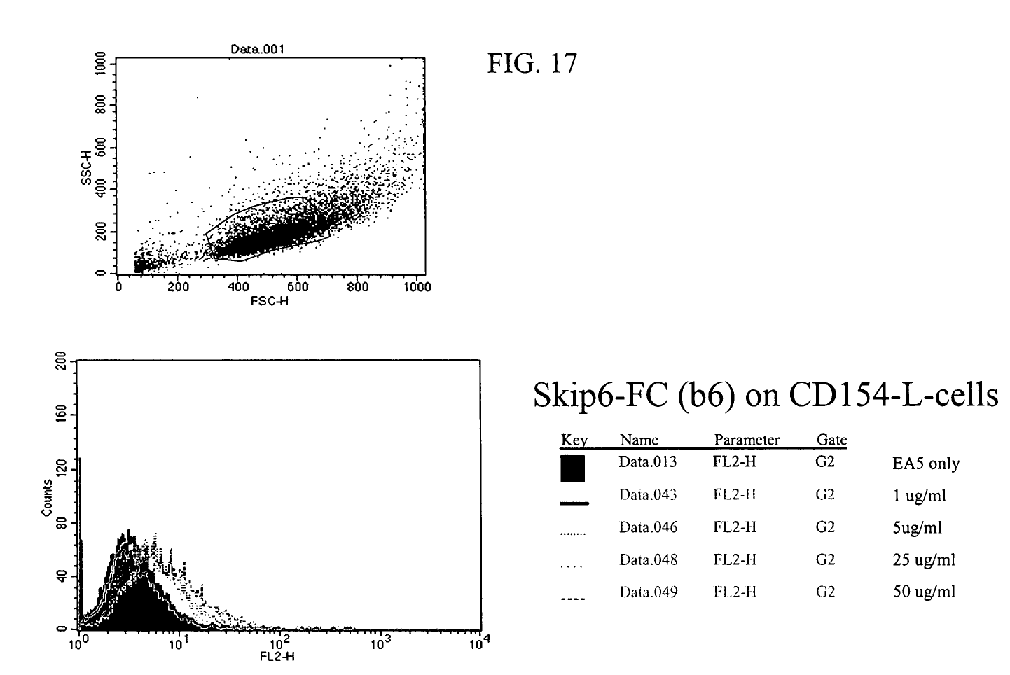

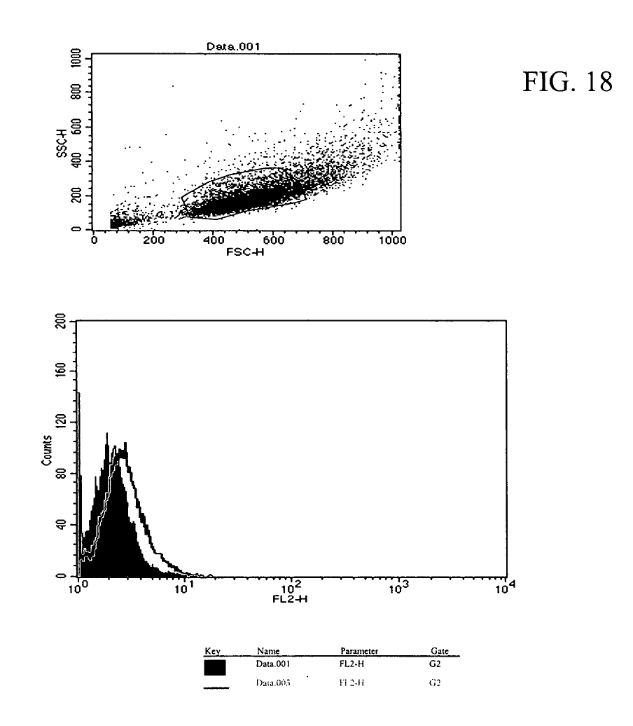

10⁶ mouse fibroblasts (stably transfected with full length human CD154) were incubated with CD40 NJ1 (FIGS. 7-8) or CD40 NJ3 variants (FIGS. 9-11). Negative control analysis using the mock of the NJ1 and NJ3 variants is shown in FIG. 12. All the proteins were incubated at a concentration of 1-50 μg/ml at 4° C. for 60 min in total volume of 100 μl (0.5×106 cells). As a control, mouse fibroblasts which do not express CD154 were used (FIGS. 6, 11, 16). sCD40 binding was detected using PE-conjugated anti CD40 non-blocking antibody (clone EA-5). Analysis was performed using BD FACsCalibur. A known CD40 soluble variant was used (in the form of a FC tagged protein) as a positive control for detecting binding to CD154 (U.S. patent application No. 60/547,422, hereby incorporated by reference as if fully set forth herein) (FIG. 17-18).

Briefly, the FACS protocol was as follows. Mouse fibroblasts were trypsinized, and washed twice in PBS; cells were centrifuged at 1500 rpm for 10 min between washes. Next, the cells were re-suspended in FACS buffer (0.2% BSA and 0.02% sodium azide diluted 1/10 in PBS) to give 5-10×10⁶ cells mL. Cells were placed in FACS test-tubes at a volume of 100 or 200 microliters per tube.

Next, CD40 proteins were added (at concentrations of [1-50 μg/ml]), optionally with other treatments or controls, to the tubes containing the mouse fibroblast cells. The tubes were vortexed to mix and incubated for 1 hr at 4° C. in the dark.

5 ml PBS was added to each tube, after which the cells were pelleted to wash. The PBS buffer was removed by vacuum aspiration to reduce the volume back to 100 mL.

Next, 2 μL (1:50) anti CD40 non-blocking antibody (EA-5 mouse IgG1 PE-conjugated, obtained from Calbiochem) or controls (same isotype PE conjugated) were added to the tubes, and incubated for 30 min at 4° C. The process of washing was then repeated with 5 mL PBS. Next, 0.5 mL PBS was added to tubes, which were read in a FACS machine (BD FACSCalibur).

Alternatively after the second washing process, it is possible to perform fixation by adding 1 ml 4% paraformaldhyde to the cells and performing FACS analysis several days after the experiment. After fixation and before FACs analysis, the washing process should be repeated.

For this experiment, the controls included performing parallel assays with: mouse fibroblasts not expressing hCD154; non-relevant Fc (EA5, which is a mouse anti-CD40 antibody, see Malmborg Hager et al., Scandinavian J. Imm. 57:517-524 (2003) or non-Fc tagged proteins or purification mock; known CD40 soluble protein (not according to the present invention, termed herein “skipping 6” and described in U.S. Pat. No. 6,720,182); secondary Ab only and isotype control only (isotype refers to an antibody control, featuring the same type of antibody but one which is not able to bind CD40).

The axes are as follows: Y=cell counts; X=log scale of fluorescence intensity. M=A marker placed above the peak of positively stained cells on the histogram plot which provide the statistics of the stained population.

The results of the FACS analysis of the CD40 NJ1 and NJ3 variants demonstrate significant binding to membrane CD154.

### Example 7

### Effect of the CD40 NJ1 and NJ3 Variants on RANTES Secretion

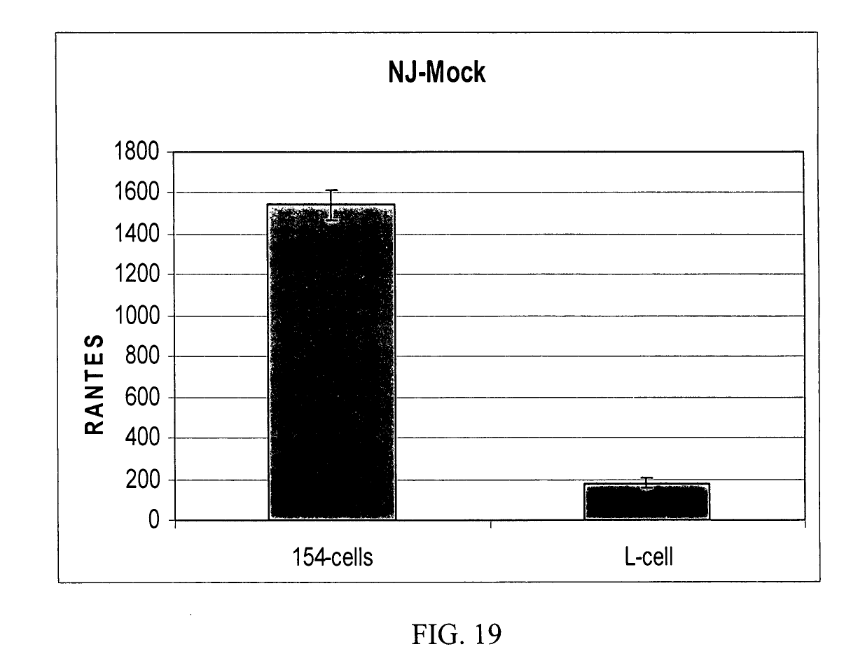

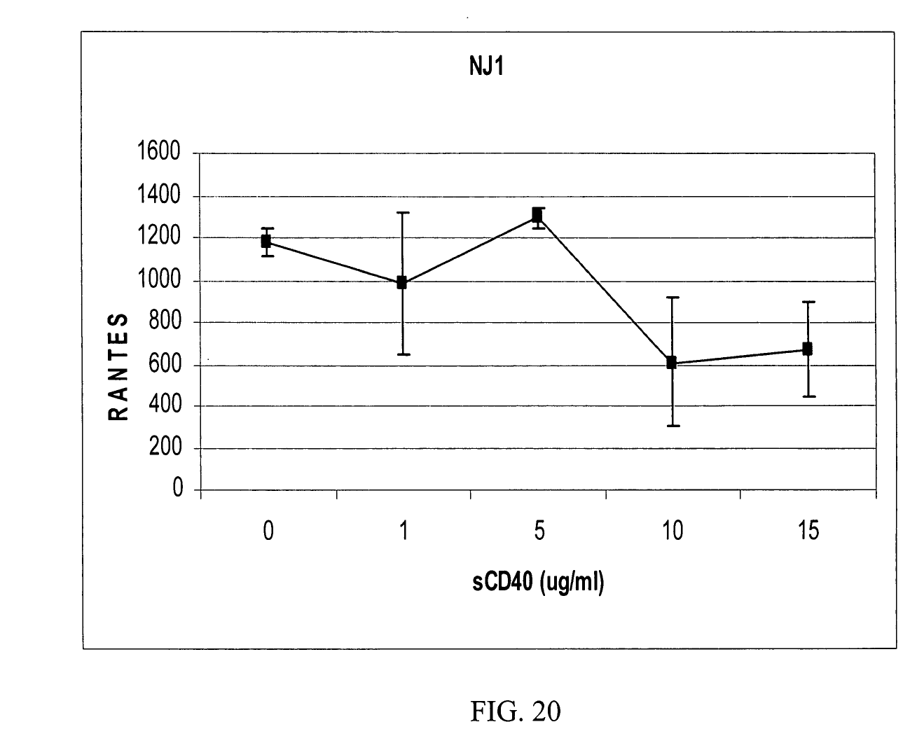

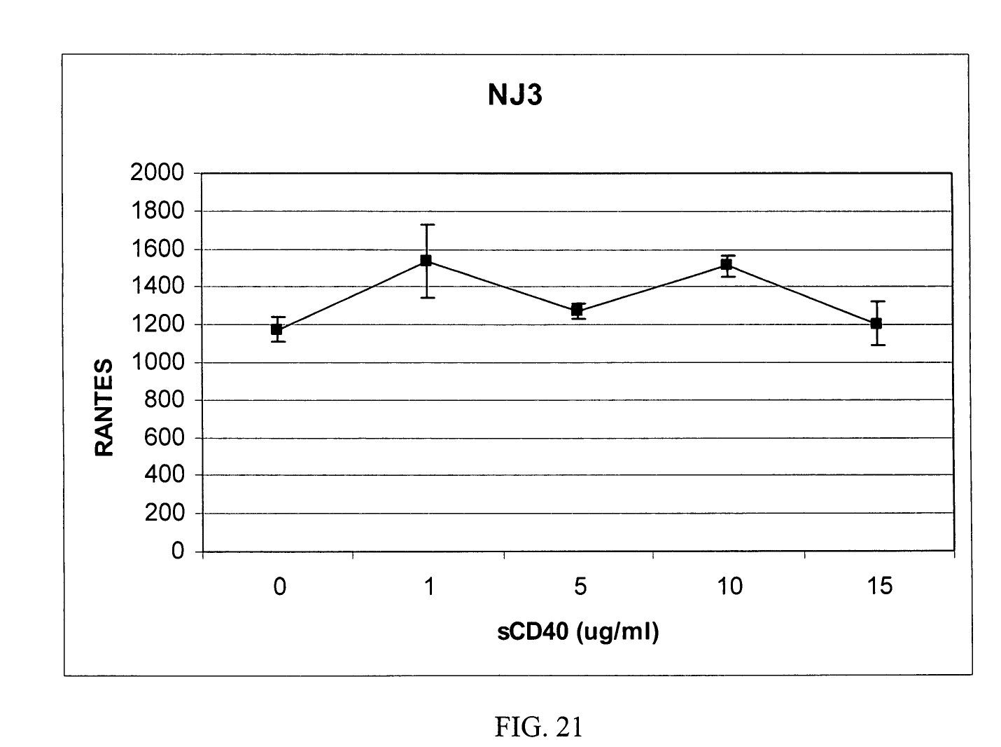

CD40 NJ1 or NJ3 variants protein was administered to a mixture of human peritoneal cells (HPMC cells), which express the CD40 receptor on their membrane, and mouse fibroblasts transfected to express the CD154 ligand. The ability of the soluble CD40 NJ1 or NJ3 variants (FIG. 20 or 21 respectively) to compete with the CD40 membrane-bound protein (i.e., wild type CD40) for binding to the CD154 ligand presented on the mouse fibroblasts was thus tested. This ability was measured by determining the resultant level of the cytokine RANTES, which is a cytokine indicative of T-cell activation, as compared to when a positive control of interferon (which raises the level of RANTES via a CD40-related pathway) was administered alone (not shown). The mock effect on RANTES secretion by mouse fibroblasts expressing or not expressing human CD154 is shown in FIG. 19.

RANTES Cell Assay Protocol

HPMC cells were grown in M199+10% FCS (Biological Industries, Bet Ha'emek, Israel), trypsinized (using trypsine from Biological Industries, Bet Ha'emek, Israel, 5 ml/75 cm² cell culture flask), and recultured into 96-well plates, to at least 80% confluence before further use.

Mouse fibroblasts/CD154-mouse fibroblasts were grown in DMEM+10% FCS (Biological Industries). Cells were trypsinized, pelleted (5 min. 500 at rpm), counted (½ vol cells+½ vol trypan blue) and resuspended in M199+10% FCS. Cells were then diluted (10,000 or 5,000 cells per ml).

CD40 proteins/antibodies were prepared in various concentrations in PBS according to the desired dose response/treatment, followed by adding the same volume of PBS to the negative and positive controls.

The CD40 protein variants were added to 100 mL of mouse fibroblasts or CD154-mouse fibroblasts in eppendorf tubes (final volume dependant on whether duplicates or triplicates were used) and incubated at R.T. (room temperature) for 1 h with rotation at 200 rpm for mixing.

During this incubation time the BPMC were prepared, by removing medium and washing cells twice, and adding 100 mL of fresh M199+10% FCS with or without 100 U/mL IFN (PeproTech, 50 U/μl).

The CD40 fibroblast cell mixtures were overlaid on the HPMC (110 ul/well) and incubated O.N (at 37° C., 5% CO₂).

100 mL were removed from the supernatant and placed into new 96 well plates, followed by diluting the samples 1/10 in M199+10% FCS and using diluted samples for the ELISA RANTES test.

### Example 8

### Treatment of Atherosclerosis by Administering NJ1 CD40 Splice Variant Protein

A human subject diagnosed with atherosclerosis is treated with a NJ1 CD40 splice variant protein to reduce the symptoms associated with the disease. An NJ1 CD40 splice variant protein is suspended in a suitable buffer for subcutaneous or intravenous delivery of the variant to the subject. Depending on the physical characteristics of the subject, e.g., height, weight, and severity of disease, the suspended protein is delivered in a dose ranging from about 1 mg/kg to 100 mg/kg by intravenous injection. Additional doses are administered as warranted from about daily to about weekly.

### Example 9

### Treatment of Atherosclerosis by Administering NJ2 CD40 Splice Variant Protein

A human subject diagnosed with atherosclerosis is treated with a NJ2 CD40 splice variant protein to reduce the symptoms associated with the disease. An NJ2 CD40 splice variant protein is suspended in a suitable buffer for subcutaneous delivery of the variant to the subject. Depending on the physical characteristics of the subject, e.g., height, weight, and severity of disease, the suspended protein is delivered in a dose ranging from about 1 mg/kg to 100 mg/kg by intravenous injection. Additional doses are administered as warrented from about daily to about weekly.

### Example 10

### Treatment of Atherosclerosis by Administering NJ3 CD40 Splice Variant Protein

A human subject diagnosed with atherosclerosis is treated with a NJ3 CD40 splice variant protein to reduce the symptoms associated with the disease. An NJ3 CD40 splice variant protein is suspended in a suitable buffer for subcutaneous or intravenous delivery of the variant to the subject. Depending on the physical characteristics of the subject, e.g., height, weight, and severity of disease, the suspended protein is delivered in a dose ranging from about 1 mg/kg to 100 mg/kg by intravenous injection. Additional doses are administered as warranted from about daily to about weekly.

### Example 11

### Treatment of Cancer by Administering NJ1 CD40 Splice Variant Protein

A subject diagnosed with colorectal cancer is treated with an NJ1 CD40 splice variant protein to reduce the symptoms associated with the disease. An NJ1 CD40 splice variant protein is suspended in a suitable buffer for subcutaneous delivery of the variant to the subject. Depending on the physical characteristics of the subject, e.g., height, weight, and severity of disease, the suspended protein is delivered in a dose ranging from about 1 mg/kg to 100 mg/kg by intravenous injection. The subject is periodically monitored by observing the change in physical symptoms and response of the cancer to treatment. Depending on the physical characteristics, additional doses are monitored from about daily to about weekly.

### Example 12

### Treatment of Cancer by Administering NJ1 CD40 Splice Variant Protein

A subject diagnosed with breast cancer is treated with an NJ2 CD40 splice variant protein to reduce the symptoms associated with the disease. An NJ2 CD40 splice variant protein is suspended in a suitable buffer for subcutaneous delivery of the variant to the subject. Depending on the physical characteristics of the subject, e.g., height, weight, and severity of disease, the suspended protein is delivered in a dose ranging from about 1 mg/kg to 100 mg/kg by intravenous injection. The subject is periodically monitored by observing the change in physical symptoms and response of the cancer to treatment. Depending on the physical characteristics, additional doses are monitored from about daily to about weekly.

### Example 13

### Treatment of Cancer by Administering NJ3 CD40 Splice Variant Protein

A subject diagnosed with lung cancer is treated with an NJ3 CD40 splice variant protein to reduce the symptoms associated with the disease. An NJ3 CD40 splice variant protein is suspended in a suitable buffer for subcutaneous or intravenous delivery of the variant to the subject. Depending on the physical characteristics of the subject, e.g., height, weight, and severity of disease, the suspended protein is delivered in a dose ranging from about 1 mg/kg to 100 mg/kg by intravenous injection. The subject is periodically monitored by observing the change in physical symptoms and response of the cancer to treatment. Depending on the physical characteristics, additional doses are monitored from about daily to about weekly.

### Example 14

### Treatment of a Chronic Inflammatory Disease by Administering NJ1 CD40 Splice Variant Protein

A subject diagnosed with inflammatory bowel syndrome is treated with an NJ1 CD40 splice variant protein to reduce the symptoms associated with the disease. An NJ1 CD40 splice variant protein is suspended in a suitable buffer for subcutaneous or intravenous delivery of the variant to the subject. Depending on the physical characteristics of the subject, e.g., height, weight, and severity of disease, the suspended protein is delivered in a dose ranging from about 1 mg/kg to 100 mg/kg by intravenous injection. The subject is periodically monitored by observing the change in physical symptoms. Depending on the physical characteristics, additional doses are monitored from about daily to about weekly.

### Example 15

### Treatment of a Chronic Inflammatory Disease by Administering NJ2 CD40 Splice Variant Protein

A subject diagnosed with multiple sclerosis treated with an NJ3 CD40 splice variant protein to reduce the symptoms associated with the disease. An NJ3 CD40 splice variant protein is suspended in a suitable buffer for subcutaneous or intravenous delivery of the variant to the subject. Depending on the physical characteristics of the subject, e.g., height, weight, and severity of disease, the suspended protein is delivered in a dose ranging from about 1 mg/kg to 100 mg/kg by intravenous injection. The subject is periodically monitored by observing the change in physical symptoms. Depending on the physical characteristics, additional doses are monitored from about daily to about weekly.

### Example 16

### Treatment of a Chronic Inflammatory Disease by Administering NJ3 CD40 Splice Variant Protein

A subject diagnosed with rheumatoid arthritis is treated with an NJ3 CD40 splice variant protein to reduce the symptoms associated with the disease. An NJ3 CD40 splice variant protein is suspended in a suitable buffer for subcutaneous delivery of the variant to the subject. Depending on the physical characteristics of the subject, e.g., height, weight, and severity of disease, the suspended protein is delivered in a dose ranging from about 1 mg/kg to 100 mg/kg by intravenous injection. The subject is periodically monitored by observing the change in physical symptoms. Depending on the physical characteristics, additional doses are monitored from about daily to about weekly.

### Example 17

### Treatment of Atherosclerosis by Gene Therapy with CD40 Splice Variant

A subject diagnosed with atherosclerosis is treated by administering a gene therapy construct capable of expressing a CD40 splice variant protein to reduce the symptoms associated with the disease. The CD40 splice variant proteins of the present invention are expressed in vivo by the expression construct. The sequences encoding the splice variant proteins of the present invention are cloned into an appropriate gene therapy vector downstream of an operable promoter. A suitable virus containing the vector construct is suspended at a concentration that results in a sufficient level of gene expression. Depending on the physical characteristics of the subject, e.g., height, weight, and severity of disease, a dose containing a particular concentration of vector is delivered by intravenous injection. The subject is periodically monitored by observing the change in physical symptoms. Depending on the physical characteristics, additional doses are monitored from about daily to about weekly.

### Example 18

### Treatment of Cancer by Gene Therapy with CD40 Splice Variant

A subject diagnosed with cancer is treated by administering a gene therapy construct capable of expressing a CD40 splice variant protein to reduce the symptoms associated with the disease. The CD40 splice variant proteins of the present invention are expressed in vivo by the expression construct. The sequences encoding the splice variant proteins of the present invention are cloned into an appropriate gene therapy vector downstream of an operable promoter. A suitable virus containing the vector construct is suspended at a concentration that results in a sufficient level of gene expression. Depending on the physical characteristics of the subject, e.g., height, weight, and severity of disease, a dose containing a particular concentration of vector is delivered by intravenous injection. The subject is periodically monitored by observing the change in physical symptoms. Depending on the physical characteristics, additional doses are monitored from about daily to about weekly.

### Example 19

### Treatment of Chronic Inflammatory Disease by Gene Therapy with CD40 Splice Variant

A subject diagnosed with chronic inflammatory disease is treated by administering a gene therapy construct capable of expressing a CD40 splice variant protein to reduce the symptoms associated with the disease. The CD40 splice variant proteins of the present invention are expressed in vivo by the expression construct. The sequences encoding the splice variant proteins of the present invention are cloned into an appropriate gene therapy vector downstream of an operable promoter. A suitable virus containing the vector construct is suspended at a concentration that results in a sufficient level of gene expression. Depending on the physical characteristics of the subject, e.g., height, weight, and severity of disease, a dose containing a particular concentration of vector is delivered by intravenous injection. The subject is periodically monitored by observing the change in physical symptoms. Depending on the physical characteristics, additional doses are monitored from about daily to about weekly.

The descriptions given are intended to exemplify, but not limit, the scope of the invention. Additional embodiments are within the claims.

1. A substantially pure protein having the amino acid sequence selected from the group consisting of:
i) SEQ ID NO:2;
ii) a fragment of SEQ ID NO:2 comprising at least 10 amino acids including at least 4 amino acids of the unique tail of SEQ ID NO:2;
iii) SEQ ID NO:4;
iv) a fragment of SEQ ID NO:4 comprising at least 10 amino acids including at least 4 amino acids of the unique tail of SEQ ID NO:4;
v) SEQ ID NO:6;
vi) a fragment of SEQ ID NO:6 comprising at least 10 amino acids including at least 4 amino acids of the unique tail of SEQ ID NO:6;
vii) SEQ ID NO:7;
viii) a fragment of SEQ ID NO:7 comprising at least 10 amino acids including at least 4 amino acids of the unique tail of SEQ ID NO:7;
ix) SEQ ID NO:8;
x) a fragment of SEQ ID NO:8 comprising at least 10 amino acids including at least 4 amino acids of the unique tail of SEQ ID NO:8;
xi) SEQ ID NO:9;
xii) a fragment of SEQ ID NO:9 comprising at least 10 amino acids including at least 4 amino acids of the unique tail of SEQ ID NO:9;
xiii) SEQ ID NO:10;
xiv) a fragment of SEQ ID NO:10 comprising at least 10 amino acids including at least 4 amino acids of the unique tail of SEQ ID NO:10; and
xv) an amino acid sequence having at least 10 amino acids and 90% identity with any one of the sequences of i)-xiv).

2. The substantially pure protein of claim 1 having the amino acid sequence selected from the group consisting of: SEQ ID NO:2; SEQ ID NO:4; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; and SEQ ID NO:17.

3. The substantially pure protein of claim 1, having the amino acid sequence of a fragment of a member of the group consisting of SEQ ID NO:2, SEQ ID NO:4 and SEQ ID NO:6, wherein the fragment has a length of any integer n equal to or between 10 and 50 amino acids, and wherein the amino acid sequence of the fragment is the amino acid sequence of a member of the respective amino acid sequence starting from any amino acid numbers 187−x; and ending at any amino acid number 188+((n−2)−x), wherein x is any integer between 0 and (n−2) or the end of the respective amino acid sequence, whichever is the lower number.

4. A pharmaceutical composition comprising a protein of claim 1 and a pharmaceutically acceptable carrier.

5. An isolated nucleic acid molecule that comprises a nucleic acid sequence that encodes a protein of claim 1.

6. A pharmaceutical composition comprising a nucleic acid molecule of claim 5 and a pharmaceutically acceptable carrier.

7. An isolated nucleic acid molecule comprising the nucleotide sequence selected from the group consisting of:
SEQ ID NO:1;
a fragment of SEQ ID NO:1 that encodes at least 10 amino acids of SEQ ID NO:2 including at least 4 amino acids of the unique tail of SEQ ID NO:2;
SEQ ID NO:3;
a fragment of SEQ ID NO:3 that encodes at least 10 amino acids of SEQ ID NO:4 including at least 4 amino acids of the unique tail of SEQ ID NO:4;
SEQ ID NO:5; and
a fragment of SEQ ID NO:5 that encodes at least 10 amino acids of SEQ ID NO:6 including at least 4 amino acids of the unique tail of SEQ ID NO:6.

8. A recombinant expression vector comprising the nucleic acid molecule of claim 7.

9. A host cell comprising the recombinant expression vector of claim 8.

10. The nucleic acid molecule of claim 7 comprising at least 12-150 nucleotides.

11. The nucleic acid molecule of claim 7 selected from the group consisting of:
a fragment of SEQ ID NO:1 comprising at least 12-150 nucleotides;
a fragment of SEQ ID NO:3 comprising at least 12-150 nucleotides; and
a fragment of SEQ ID NO:5 comprising at least 12-150 nucleotides.

12. The nucleic acid molecule of claim 7 comprising at least 15-50 nucleotides.

13. The nucleic acid molecule of claim 7 selected from the group consisting of:
a fragment of SEQ ID NO:1 comprising at least 15-50 nucleotides;
a fragment of SEQ ID NO:3 comprising at least 15-50 nucleotides; and
a fragment of SEQ ID NO:5 comprising at least 15-50 nucleotides.

14. The nucleic acid molecule of claim 7 comprising at least 18-30 nucleotides.

15. The nucleic acid molecule of claim 7 selected from the group consisting of:
a fragment of SEQ ID NO:1 comprising at least 18-30 nucleotides;
a fragment of SEQ ID NO:3 comprising at least 18-30 nucleotides; and
a fragment of SEQ ID NO:5 comprising at least 18-30 nucleotides.

16. An isolated antibody that binds to an epitope on SEQ ID NO:2 that includes at least 4 amino acids from the unique tail of SEQ ID NO:2; an epitope on SEQ ID NO:4 that includes at least 4 amino acids from the unique tail of SEQ ID NO:4; an epitope on SEQ ID NO:6 that includes at least 4 amino acids from the unique tail of SEQ ID NO:6; an epitope on SEQ ID NO:7 that includes at least 4 amino acids from the unique tail of SEQ ID NO:7; an epitope on SEQ ID NO:8 that includes at least 4 amino acids from the unique tail of SEQ ID NO:8; an epitope on SEQ ID NO:9 that includes at least 4 amino acids from the unique tail of SEQ ID NO:9; or an epitope on SEQ ID NO:10 that includes at least 4 amino acids from the unique tail of SEQ ID NO:10;
wherein the isolated antibody does not bind to an epitope consisting of an amino acid fragment of SEQ ID NO:24.

17. The antibody of claim 16 wherein said antibody is a monoclonal antibody.

18. A method of detecting the presence of a protein in a sample, the method comprising
contacting a sample with an antibody of claim 16; and
determining if said antibody is bound to protein in said sample,
wherein binding of said antibody to protein in said sample is indicative of the presence of a protein selected from the group consisting of SEQ ID NO:2, SEQ ID NO:4, SEQ ID NO:6, SEQ ID NO:7, SEQ ID NO:8, SEQ ID NO:9 and SEQ ID NO:10 in said sample.

19. The method of claim 18 wherein said sample is body fluid.

20. The method of claim 19 wherein said body fluid is blood.

21. A kit for detecting the presence of a protein in a sample, the protein selected from the group consisting of SEQ ID NO:2, SEQ ID NO:4, SEQ ID NO:6, SEQ ID NO:7, SEQ ID NO:8, SEQ ID NO:9 and SEQ ID NO:10 comprising a container that comprises an antibody of claim 16 and a container that comprises a positive control sample or a container that comprises a negative control sample.

22. A method of detecting whether an individual is expressing a protein, the method comprising
detecting in a sample from the individual a transcript that encodes a protein selected from the group consisting of SEQ ID NO:2, SEQ ID NO:4, SEQ ID NO:6, SEQ ID NO:7, SEQ ID NO:8, SEQ ID NO:9 and SEQ ID NO:10,
wherein detection of the transcript in a sample is indicative of expression of said protein by said individual.

23. The method of claim 22 wherein the transcript is detected using polymerase chain reaction.

24. The method of claim 22 wherein said sample is body fluid.

25. The method of claim 24 wherein said body fluid is blood.

26. A method of modulating CD40-CD 154 interactions in an individual, the method comprising administering to said individual a protein of claim 1 in an amount effective to modulate CD40-CD154 interactions in said individual.

27. The method of claim 26 wherein the individual is suspected of suffering from chronic inflammatory disease.

28. The method of claim 26 wherein the individual is suspected of suffering from a condition selected from the group consisting of cancer, atherosclerosis, and acute injury.

29. A method of modulating CD40-CD 154 interactions in an individual, the method comprising administering to said individual a nucleic acid molecule that comprises a coding sequence that encodes a protein of claim 1,
wherein said coding sequence is operatively linked to regulatory sequences necessary for expression in the individual, and
wherein the protein is produced by expression of the coding sequence in an amount effective to modulate CD40-CD154 interactions in the individual.

30. The method of claim 29 wherein the individual is suspected of suffering from chronic inflammatory disease.

31. The method of claim 29 wherein the individual is suspected of suffering from a condition selected from the group consisting of cancer, atherosclerosis, and acute injury.

32. A purified polypeptide, comprising a first amino acid sequence at least 90% identical to amino acids 1-187 of wild type CD40 (SEQ ID NO: 24), and a second amino acid sequence being at least 90% identical to a polypeptide having the sequence ESWTMGPGESLGRWELKGEMRHTGTLDGKKGRGGSLGVWYHSSATYLGSLGKSLPLS (SEQ ID NO:11), wherein said first and said second amino acid sequences are contiguous and in a sequential order.

33. A fragment of the polypeptide of claim 32, wherein the fragment has a length of any integer n equal to or between 10 and 50 amino, and wherein the amino acid sequence of the fragment is the amino acid sequence of SEQ ID NO:2 starting at any of amino acid numbers 187−x to 187, and ending at any of amino acid number 188+((n−2)−x), wherein x is any integer between 0 and (n−2) or the end of SEQ ID NO:2, whichever is the lower number, and wherein at least two of the amino acids of the fragment are glycine and glutamate.

34. The polypeptide of claim 32, wherein the second amino acid sequence is at least 95% identical to the polypeptide having the sequence ESWTMGPGESLGRWELKGEMRHTGTLDGKKGRGGSLGVWYHSSATYLGSLGKSLPLS (SEQ ID NO:11).

35. The polypeptide of claim 32, wherein the second amino acid sequence is at least 98% identical to the polypeptide having the sequence ESWTMGPGESLGRWELKGEMRHTGTLDGKKGRGGSLGVWYHSSATYLGSLGKSLPLS (SEQ ID NO:11).

36. The polypeptide of claim 32, wherein the second amino acid sequence is at least 99% identical to the polypeptide having the sequence ESWTMGPGESLGRWELKGEMRHTGTLDGKKGRGGSLGVWYHSSATYLGSLGKSLPLS (SEQ ID NO:11).

37. A purified polypeptide comprising an amino acid sequence at least 90% identical to a polypeptide having the sequence ESWTMGPGESLGRWELKGEMRHTGTLDGKKGRGGSLGVWYHSSATYLGSLGKSLPLS (SEQ ID NO:11).

38. A purified polypeptide comprising a first amino acid sequence being at least 90% identical to amino acids 1-187 of wild type CD40 (SEQ ID NO: 24), and a second amino acid sequence being at least 75% identical to a polypeptide having the sequence LGLE (SEQ ID NO:12), wherein said first and said second amino acid sequences are contiguous and in a sequential order.

39. A fragment of the polypeptide of claim 38, wherein the fragment has a length of any integer n equal to or between 10 and 50 amino, and wherein the amino acid sequence of the fragment is the amino acid sequence of SEQ ID NO:4 starting at any of amino acid numbers (187−x) to 187, and ending at any of amino acid number 188+((n−2)−x), wherein x is any integer between 0 and (n−2) or the end of SEQ ID NO:4, whichever is the lower number, and wherein at least two of the amino acids of the fragment are glycine and leucine.

40. A purified polypeptide, comprising a first amino acid sequence being at least 90% identical to amino acids 1-187 of wild type CD40 (SEQ ID NO: 24), and a second amino acid sequence being at least 90% identical to a polypeptide having the sequence ESWTMGPGESLGRSPGSAESPGGDPHHLRDPVCHPLGAGLYQKGGQEANQ (SEQ ID NO:13), wherein said first and said second amino acid sequences are contiguous and in a sequential order.

41. A fragment of the polypeptide of claim 40, wherein the fragment has a length of any integer n equal to or between 10 and 50 amino, and wherein the amino acid sequence of the fragment is the amino acid sequence of SEQ ID NO:6 starting at any of amino acid numbers (187−x) to 187, and ending at any of amino acid numbers 188+((n−2)−x), wherein x is any integer between 0 and (n−2) or the end of SEQ ID NO:6, whichever is the lower number, and wherein at least two of the amino acids of the fragment are glycine and glutamate.

42. The polypeptide of claim 40, wherein the second amino acid sequence is at least 95% identical to the polypeptide having the sequence ESWTMGPGESLGRSPGSAESPGGDPHHLRDPVCHPLGAGLYQKGGQEANQ (SEQ ID NO:13).

43. The polypeptide of claim 40, wherein the second amino acid sequence is at least 98% identical to the polypeptide having the sequence ESWTMGPGESLGRSPGSAESPGGDPHHLRDPVCHPLGAGLYQKGGQEANQ (SEQ ID NO:13).

44. The polypeptide of claim 40, wherein the second amino acid sequence is at least 99% identical to the polypeptide having the sequence ESWTMGPGESLGRSPGSAESPGGDPHHLRDPVCHPLGAGLYQKGGQEANQ (SEQ ID NO:13).

45. A purified polypeptide comprising an amino acid sequence at least 90% identical to a polypeptide having the sequence ESWTMGPGESLGRSPGSAESPGGDPHHLRDPVCHPLGAGLYQKGGQEANQ (SEQ ID NO:13).

46. An antibody or an antibody fragment being capable of specifically binding to a CD40 variant polypeptide comprising an amino acid sequence of claim 3, wherein said antibody or antibody fragment does not bind to an epitope consisting of an amino acid fragment of SEQ ID NO:24.

47. An antibody or an antibody fragment being capable of specifically binding to a CD40 variant polypeptide comprising an amino acid sequence of claim 32, wherein said antibody or antibody fragment does not bind to an epitope consisting of an amino acid fragment of SEQ ID NO:24.

48. An antibody or an antibody fragment being capable of specifically binding to a CD40 variant polypeptide comprising an amino acid sequence of claim 37, wherein said antibody or antibody fragment does not bind to an epitope consisting of an amino acid fragment of SEQ ID NO:24.

49. An antibody or an antibody fragment being capable of specifically binding to a CD40 variant polypeptide comprising an amino acid sequence of claim 38, wherein said antibody or antibody fragment does not bind to an epitope consisting of an amino acid fragment of SEQ ID NO:24.

50. An antibody or an antibody fragment being capable of specifically binding to a CD40 variant polypeptide comprising an amino acid sequence of claim 40, wherein said antibody or antibody fragment does not bind to an epitope consisting of an amino acid fragment of SEQ ID NO:24.

51. An antibody or an antibody fragment being capable of specifically binding to a CD40 variant polypeptide comprising an amino acid sequence of claim 45, wherein said antibody or antibody fragment does not bind to an epitope consisting of an amino acid fragment of SEQ ID NO:24.


SEQUENCE LISTING (43 sequences)

SEQ ID NO: 1 - DNA, 1088 bp, Homo sapiens
ggctggggcaggggagtcagcagaggcctcgctcgggcgcccagtggtcctgccgcctggtctcacctcgccatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtgagtcctggacaatgggccctggagaaagcctaggaaggtgggaactgaagggggagatgaggcacacaggaacactggatgggaaaaaggggaggggaggcagtttgggggtgtggtatcacagctctgccacttatcttgggagtctgggcaaatcacttcccctctcttagcctcagtttcttcatctgtaaaatgggatgataacagcacttccttagtaggttttgattttagagtgagaaggttggcctacagtaaagatcagataatgtaaatcagtgaaaaaggtcaggggtaagaaaattacattctctttacctaacgctaaatgaccagttaatgggtgcagcacaccaacatggtacatgtatacatatgtaacaaacctgcacattatgcacatgtaccctaaagcttaaagtataataataataaaatttaaaaaaacgaa

SEQ ID NO: 2 - protein, 244 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGESWTMGPGESLGRWELKGEMRHTGTLDGKKGRGGSLGVWYHSSATYLGSLGKSLPLS

SEQ ID NO: 3 - DNA, 1813 bp, Homo sapiens
ggctggggcaggggagtcagcagaggcctcgctcgggcgcccagtggtcctgccgcctggtctcacctcgccatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtgggctgggactagaatgaggtgagcaaggcacttgccctcgggcgcaatatttaagaaggtgccataaaagtgtagtaatcaaggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaagtttggtggtggtggtgttggggtatggtttagtaatatccaccagaccttccgatccagcagtttggtgcccagagaggcatcatggtggcttccctgcgcccaggaagccatatacacagatgcccattgcagcattgtttgtgatagtgaacaactggaagctgcttaactgtccatcagcaggagactggctaaataaaattagaatatatttatacaaacagaatctcaaaaacactgttgagtaaggaaaaaaaggcatgctgctgaatgatgggtatggaactttttaaaaaagtacatgcttttatgtatgtatattgcctatggatatatgtataaatacaatatgcatcatatattgatataacaagggttctggaagggtacacagaaaacccacagctcgaagagtggtgacgtctggggtggggaagaagggtctgggggagggttggttaaagggagatttggctttcccataatgcttcatcatttttcccaaaaggagagtgaattcacataatgcttatgtaattaaaaaatcatcaaacatgtaaaaagaaaa

SEQ ID NO: 4 - protein, 191 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGLGLE

SEQ ID NO: 5 - DNA, 1770 bp, Homo sapiens
ggctggggcaggggagtcagcagaggcctcgctcgggcgcccagtggtcctgccgcctggtctcacctcgccatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtgagtcctggacaatgggccctggagaaagcctaggaaggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaagtttggtggtggtggtgttggggtatggtttagtaatatccaccagaccttccgatccagcagtttggtgcccagagaggcatcatggtggcttccctgcgcccaggaagccatatacacagatgcccattgcagcattgtttgtgatagtgaacaactggaagctgcttaactgtccatcagcaggagactggctaaataaaattagaatatatttatacaaacagaatctcaaaaacactgttgagtaaggaaaaaaaggcatgctgctgaatgatgggtatggaactttttaaaaaagtacatgcttttatgtatgtatattgcctatggatatatgtataaatacaatatgcatcatatattgatataacaagggttctggaagggtacacagaaaacccacagctcgaagagtggtgacgtctggggtggggaagaagggtctgggggagggttggttaaagggagatttggctttcccataatgcttcatcatttttcccaaaaggagagtgaattcacataatgcttatgtaattaaaaaatcatcaaacatgtaaaaagaaaa

SEQ ID NO: 6 - protein, 237 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGESWTMGPGESLGRSPGSAESPGGDPHHLRDPVCHPLGAGLYQKGGQEANQ

SEQ ID NO: 7 - protein, 156 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIAVRPKTWLCNRQAQTRLMLSVV

SEQ ID NO: 8 - protein, 229 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGESWTMGPGESLGRSPGSAESPGGDPHHLRDPVCHPLGAGLYQ

SEQ ID NO: 9 - protein, 42 aa, Homo sapiens
  VARIANT (38)..(38): Wherein Xaa is any amino acid.
ESRGEWPCQVFGKQGTGERLRHAGTLTGIGVRPRGSLXYSTL

SEQ ID NO: 10 - protein, 160 aa, Homo sapiens
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMAVRIRTWRSYRKERVRLMSSVVPRIG

SEQ ID NO: 11 - protein, 57 aa, Homo sapiens
ESWTMGPGESLGRWELKGEMRHTGTLDGKKGRGGSLGVWYHSSATYLGSLGKSLPLS

SEQ ID NO: 12 - protein, 4 aa, Homo sapiens
LGLE

SEQ ID NO: 13 - protein, 50 aa, Homo sapiens
ESWTMGPGESLGRSPGSAESPGGDPHHLRDPVCHPLGAGLYQKGGQEANQ

SEQ ID NO: 14 - protein, 21 aa, Homo sapiens
VRPKTWLCNRQAQTRLMLSVV

SEQ ID NO: 15 - protein, 42 aa, Homo sapiens
ESWTMGPGESLGRSPGSAESPGGDPHHLRDPVCHPLGAGLYQ

SEQ ID NO: 16 - protein, 42 aa, Homo sapiens; identical to SEQ ID NO: 9
  misc_feature (38)..(38): Xaa can be any naturally occurring amino acid

SEQ ID NO: 17 - protein, 25 aa, Homo sapiens
VRIRTWRSYRKERVRLMSSVVPRIG

SEQ ID NO: 18 - DNA, 24 bp, Homo sapiens
ggcactgtacgagtgaggcctgtg

SEQ ID NO: 19 - DNA, 23 bp, Homo sapiens
tgcctcatctcccccttcagttc

SEQ ID NO: 20 - DNA, 24 bp, Homo sapiens
ttgctcacctcattctagtcccag

SEQ ID NO: 21 - DNA, 22 bp, Homo sapiens
tgttggagccaggaagatcgtc

SEQ ID NO: 22 - DNA, 22 bp, Homo sapiens
gagtcctggacaatgggccctg

SEQ ID NO: 23 - DNA, 910 bp, Homo sapiens
gcctcgctcgggcgcccagtggtcctgccgcctggtctcacctcgccatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacg

SEQ ID NO: 24 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 25 - protein, 20 aa, Homo sapiens
CELKGEMRHTGTLDGKKGRG

SEQ ID NO: 26 - protein, 15 aa, Homo sapiens
CGESWTMGPGESLGR

SEQ ID NO: 27 - DNA, 31 bp, Homo sapiens
  misc_feature (30)..(31): Wherein n is a or t or c or g.
ggggacaagtttgtacaaaaaagcaggctnn

SEQ ID NO: 28 - DNA, 52 bp, Homo sapiens
ggggacaagtttgtacaaaaaagcaggctccatggttcgtctgcctctgcag

SEQ ID NO: 29 - DNA, 30 bp, Homo sapiens
  misc_feature (30)..(30): Wherein n is a or t or c or g.
ggggaccactttgtacaagaaagctgggtn

SEQ ID NO: 30 - DNA, 51 bp, Homo sapiens
ggggaccactttgtacaagaaagctgggtactaagagaggggaagtgattt

SEQ ID NO: 31 - DNA, 51 bp, Homo sapiens
ggggaccactttgtacaagaaagctgggtattattggttggcttcttggcc

SEQ ID NO: 32 - DNA, 21 bp, Homo sapiens
cgcgttaacgctagcatggat

SEQ ID NO: 33 - DNA, 23 bp, Homo sapiens
cacagagttttagagactacaat

SEQ ID NO: 34 - DNA, 20 bp, Homo sapiens
caccatctgcacctgtgaag

SEQ ID NO: 35 - DNA, 30 bp, Homo sapiens
  misc_feature (1)..(4): Wherein n is a or t or c or g.
nnnnagatcttaagagaggggaagtgattt

SEQ ID NO: 36 - DNA, 29 bp, Homo sapiens
  misc_feature (1)..(4): Wherein n is a or t or c or g.
nnnnagatcttattctagtcccagcccac

SEQ ID NO: 37 - DNA, 29 bp, Homo sapiens
  misc_feature (1)..(4): Wherein n is a or t or c or g.
nnnnagatcttattggttggcttcttggc

SEQ ID NO: 38 - protein, 4 aa, Homo sapiens
VCGE

SEQ ID NO: 39 - protein, 4 aa, Homo sapiens
CGES

SEQ ID NO: 40 - protein, 4 aa, Homo sapiens
GESW

SEQ ID NO: 41 - protein, 4 aa, Homo sapiens
VCGL

SEQ ID NO: 42 - protein, 4 aa, Homo sapiens
CGLG

SEQ ID NO: 43 - protein, 4 aa, Homo sapiens
GLGL